SEQUENCE LISTING (6 sequences)

SEQ ID NO: 1 - protein, 91 aa, Homo sapiens
ARILRAPESHNVTFGSFVTLHCTATGIPVPTITWIENGNAVSSGSIQESVKDRVIDSRLQLFITKPGLYTCIATNKHGEKFSTAKAAATIS

SEQ ID NO: 2 - protein, 91 aa, Mus musculus
ARILRAPESHNVTFGSFVTLRCTAIGIPVPTISWIENGNAVSSGSIQESVKDRVIDSRLQLFITKPGLYTCIATNKHGEKFSTAKAAATVS

SEQ ID NO: 3 - DNA, 20 bp, Artificial Sequence
  Description of Artificial Sequence Synthetic oligonucleotide
tgctcatatctaaatgcgat

SEQ ID NO: 4 - DNA, 20 bp, Artificial Sequence
  Description of Artificial Sequence Synthetic oligonucleotide
gcactccatggcatctggaa

SEQ ID NO: 5 - DNA, 20 bp, Artificial Sequence
  Description of Artificial Sequence Synthetic oligonucleotide
gagcataaatgttctagact

SEQ ID NO: 6 - DNA, 20 bp, Artificial Sequence
  Description of Artificial Sequence Synthetic oligonucleotide
ctccatggcatctggaaggg

(19) United States
(12) Patent Application Publication
(10) Pub. No.: US 2023/0304012 A1
(43) Pub. Date: Sep. 28, 2023

(54) MUSCLE REGENERATION AND GROWTH

(71) Applicant: Brown University, Providence, RI (US)

(72) Inventors: Justin R. FALLON, Providence, RI (US); Laura A. MADIGAN, Pawtucket, RI (US); Ashley E. WEBB, Providence, RI (US); Beth MCKECHNIE, Attleboro, MA (US); Lauren FISH, East Greenwich, RI (US); Diego JAIME, Providence, RI (US); John PAGE, Storrs, CT (US)

(21) Appl. No.: 17/769,165

(22) PCT Filed: Oct. 16, 2020
(86) PCT No.: PCT/US2020/055971

§ 371 Date: Apr. 14, 2022

Related U.S. Application Data
(60) Provisional application No. 62/915,980, filed on Oct. 16, 2019.
(60) Provisional application No. 63/011,876, filed on Apr. 17, 2020.

(51) Int. Cl. C12N 15/113; C07K 16/40; A61K 38/46; A61K 31/7088; A61P 21/00

(52) U.S. Cl. CPC C12N 15/1137; C07K 16/40; A61K 38/465; A61K 31/7088; A61P 21/00; C12N 2310/14; C12N 2310/321

(57) ABSTRACT

Methods and compositions for increasing muscle regeneration and for preventing or treating diseases, disorders or conditions associated with neuromuscular dysfunction, are provided herein.

### STATEMENT REGARDING FEDERALLY SPONSORED RESEARCH OR DEVELOPMENT

This invention was developed with the following funding: NIH Grant: T32 award 5T32AG041688-07 and U01 grant: NIH 1U01NS064295-04 awarded by the National Institutes of Health. The government has certain rights in the invention.

### BACKGROUND

Skeletal muscle has the ability to regenerate after injury. Muscle regeneration is dependent upon resident stem cells, referred to as muscle satellite cells. In mature muscle tissue, satellite cells constitute a small, scattered population of mitotically and physiologically quiescent cells. Satellite cells are also implicated in normal muscle growth and maintenance throughout life, indicating that they could be exploited to treat muscle wasting conditions.

Skeletal muscle makes up about 35% of body weight and is essential for metabolism, locomotion, and breathing, which highlights its importance in human health. Muscle wasting reduces mobility, metabolism, and quality of life for the majority of cancer patients, elderly patients, and many others with no history of neuromuscular dysfunction. In addition, muscular dystrophies are an often fatal group of genetic diseases leading to severe muscle loss, including Duchenne Muscular Dystrophy which affects children. A roadblock to development of treatments is that the signaling that regulates satellite cells and muscle regeneration is poorly understood. Accordingly, there is a need for compositions and methods for muscle regeneration and growth.

### SUMMARY

Bone morphogenetic protein (BMP) signaling regulates skeletal muscle stem cell activity in both normal and pathological states. Embodiments of the present invention provide technologies for modulating BMP signaling. Among other things, the present disclosure provides an insight that important interactions between BMP and muscle-specific tyrosine kinase protein (MuSK) are mediated by the third immunoglobulin domain (Ig3) of MuSK. The present disclosure demonstrates that BMP signaling is perturbed in the absence of the Ig3 domain. Furthermore, the present disclosure teaches that modulating BMP signaling via pharmacological intervention targeting MuSK provides an attractive method for enhancing muscle stem cell activity and increasing muscle regeneration.

The present disclosure provides technologies for increasing level and/or activity of MuSK form(s) that functionally participate in muscle regeneration, including, in some embodiments, by reducing alternative splicing that would otherwise generate MuSK form(s) that do not so participate. In some embodiments, such increase is in a relevant tissue such as muscle. Alternatively or additionally, in some embodiments, such increase is in a tissues such as a brain tissue (e.g., hippocampal and/or subventricular) and/or lung tissue.

In some embodiments, the present disclosure provides technologies for agonizing Ig3-MuSK, for example by increasing level and/or activity of one or more forms of MuSK whose Ig3 domain is altered (e.g., mutated, blocked, removed, etc.) for example so that it fails to effectively participate in interaction(s) with BMP. In some embodiments, the present disclosure provides technologies for reducing Ig3⁺ MuSK, for example by reducing level and/or activity of one or more forms of MuSK whose Ig3 domain effectively participates in interaction(s) with BMP. Alternatively or additionally, in some embodiments, the present disclosure provides technologies that reduce level and/or activity of a MuSK Ig3/BMP complex (e.g., that inhibit formation of, disrupt, and/or otherwise antagonize such complex).

In some embodiments, the present disclosure provides a MuSK muscle regeneration agonizing agent that targets the MuSK Ig3 domain and/or BMP so that level and/or activity of a MuSK/BMP complex is reduced. In some such embodiments, such a MuSK muscle regeneration agonizing agent inhibits formation of and/or disrupts such complex(es). In some embodiments, such a MuSK muscle regeneration agonizing agent competes with BMP for binding to MuSK Ig3 and/or competes with MuSK Ig3 for binding to BMP.

In some embodiments, an agent that targets the MuSK Ig3 domain and/or BMP, as described herein (e.g., a MuSK muscle regeneration agonizing agent), so that level and/or activity of a MuSK/BMP complex is reduced is useful in contexts of muscle regeneration and/or of muscle growth. In some embodiments, provided agent(s) may enhance muscle growth. In some embodiments, muscle growth occurs in uninjured tissue. In some embodiments, muscle growth occurs in injured tissue. In some embodiments, enhanced and/or increased muscle growth is determined by a decrease in satellite cell number and/or increase in muscle fiber size. In this regard, muscle growth can be characterized by a decrease in satellite cell number and/or increase in muscle fiber size, which is indicative of satellite cells differentiating and fusing into/augmenting existing muscle fibers and forming new muscle fibers.

Embodiments of the present invention provide methods of enhancing muscle regeneration and/or growth, for example in a subject in need thereof, by administering a composition that downregulates MuSK Ig3 domain protein expression, MuSK Ig3 domain gene expression, and/or MuSK Ig3 activation of BMP signaling, thereby upregulating muscle satellite cells which results in enhancement of muscle regeneration and/or growth. In some embodiments, such a composition can comprise and/or deliver a MuSK Ig3-targeted blocking antibody, a MuSK Ig3-targeted exon-skipping oligonucleotide, a MuSK Ig3-targeted CRISPR/Cas9, a MuSK Ig3-targeted siRNA, a MuSK Ig3-targeted small molecule, and/or a MuSK Ig3-targeted shRNA.

In some embodiments, a subject of interest can be at risk of, or afflicted with, a disease or disorder including, but not limited to, neuromuscular dysfunction, neurodegenerative disorder, cardiac disorder (e.g., myocardial infarction, cardiomyopathy), or genetic diseases characterized by muscle wasting. Alternatively or additionally, in some embodiments, a subject of interest can be at risk of, or afflicted with a disease or disorder associated with lung damage, including, for example, idiopathic pulmonary fibrosis (IPF), acute respiratory distress syndrome (ARDS), pneumonia, and/or certain infections, including viral infections including coronaviral infections such as COVID-19.

Exemplary neuromuscular dysfunctions or disorders that can be treated by technologies of the present invention include, but are not limited to, Becker muscular dystrophy, Congenital muscular dystrophy, Distal muscular dystrophy, Duchenne muscular dystrophy, Emery-Dreifuss muscular dystrophy, Facioscapulohumeral muscular dystrophy, Limb-girdle muscular dystrophy, Myotonic muscular dystrophy, and Oculo-pharyngeal muscular dystrophy.

In some embodiments, enhancing muscle growth is used in the context of treating a disease or disorder associated with muscle atrophy or muscle wasting. Muscle atrophy or muscle wasting may be observed in connection with various diseases and conditions described herein, such as neuromuscular disorders, or direct or indirectly caused by prolonged inactivity, bed rest, hospitalization, aging, malnutrition, cancer cachexia, chronic inflammatory diseases, etc. Example chronic inflammatory diseases include rheumatoid arthritis, chronic heart failure, and chronic obstructive pulmonary disease (COPD).

Duration of hospitalization and type and severity of the illness can affect the extent of muscle wasting in a subject, and muscle wasting is common in patients suffering from sepsis, organ failure, hyperglycemia, and diseases associated with chronic and systemic inflammation or oxidative stress. Additionally, hospitalization requiring complete immobilization/bed rest contributes significantly to muscle wasting.

Additional disorders associated with muscle atrophy/wasting include disorders associate with decreased mobility, such as rheumatoid arthritis, osteoarthritis, and injury. Powers et al. (2016). Thus, in some embodiments, the present disclosure provides technologies for preventing/treating muscle wasting or muscle atrophy related to or as a result of a number of diseases or conditions described herein.

In some embodiments, methods of the present invention can also be used when a subject is in need of enhanced muscle regeneration and muscle growth following surgery, trauma and/or prolonged immobilization (e.g., from bed-rest or casting). As muscle stem cell activity is known to decrease with age, methods of the present invention can also be used to prevent or reverse sarcopenia in patients that are otherwise healthy and could lead to significant improvements in quality of life and autonomy.

Embodiments of the present invention also provide methods of preventing or treating muscle fibrosis, e.g., in a subject in need thereof, by administering a composition that downregulates the MuSK Ig3 domain protein expression, the MuSK Ig3 domain gene expression, and/or the MuSK Ig3 activation of BMP signaling, which prevents or reduces the accumulation of extracellular matrix within the extracellular space of the muscle. The composition can comprise a MuSK Ig3-targeted blocking antibody, a MuSK Ig3-targeted exon skipping oligonucleotide, a MuSK Ig3-targeted CRISPR/Cas9, a MuSK Ig3-targeted siRNA, a MuSK Ig3-targeted small molecule, and/or a MuSK Ig3-targeted shRNA.

The subject can at risk of, or afflicted with, muscle fibrosis resulting from a disease or condition including, but not limited to, trauma, heritable disease, muscle disorder, and aging. The trauma can result from, for example, radiation treatment, crush injury, laceration, and amputation. The heritable disease or muscle disorder include, but are not limited to, Congenital Muscular Dystrophy, Duchenne Muscular Dystrophy, Becker's Muscular Dystrophy; Amyotrophic Lateral Sclerosis (ALS), and age-associate sarcopenia.

In some embodiments, the present disclosure provides model systems that can be used to screen, validate, characterize, assess, and/or identify agents that downregulate the MuSK Ig3 domain protein expression, the MuSK Ig3 domain gene expression, and/or the MuSK Ig3 activation of BMP signaling, which prevents or reduces the accumulation of extracellular matrix within the extracellular space of the muscle. In some embodiments, the model system is an artificially engineered cell line. In some embodiments, the model system is a genetically engineered mouse model. In some embodiments, the mouse model includes ΔIg3-MuSK mice.

### BRIEF DESCRIPTION OF THE DRAWING

For the purpose of illustration, certain embodiments of the present invention are shown in the drawings described below. Like numerals in the drawings indicate like elements throughout. It should be understood, however, that the invention is not limited to the precise arrangements, dimensions, and instruments shown. In the drawings:

FIG. 1 illustrates MuSK expression in satellite cells. FIG. 1A shows protein expression markers of satellite cells. FIG. 1B shows structures of full length MuSK and ΔIg3-MuSK. FIG. 1C shows immunohistochemistry (IHC) of isolated, intact WT myofibers stained immediately (quiescent), or after 24 hours in culture (activated). These IHC data show that activated, but not quiescent satellite cells express detectable MuSK. ΔIg3-MuSK satellite cells have a similar expression pattern (not shown).

FIG. 2 shows the structures of full length MuSK and ΔIg3-MuSK in relation to the BMP receptor. The Ig3 domain is necessary for high affinity BMP binding. ΔIg3-MuSK has reduced MuSK-BMP activity and muscle regeneration and growth is accelerated in muscles expressing ΔIg3-MuSK.

FIG. 3 shows Wnt11 transcription in primary myotubes from neo-natal mice treated with or without 25 ng/mL BMP4 for 2 hours. MuSK-dependent BMP4-induced Wnt11 transcription was reduced in ΔIg3-MuSK myotubes as determined by qRT-PCR. These data show a 5.8-fold increase vs. 2.6-fold increase, n=6 replicate cultures in two separate experiments, T-test, p=0.0007.

FIG. 4 illustrates characterization of satellite cells (SCs) in uninjured muscle and in regenerating muscle 5, 7, and 14 days post injury (dpi). FIG. 4A provides a diagrammatic illustration of the experiment protocol. The tibialis anterior muscle of 5-8 month old males was injured. 5-ethynyl-2′-deoxyuridine (EdU) was administered via intraperitoneal (i.p.) injection 4, 6, or 13 dpi, and muscle tissue was harvested 5, 7, or 14 dpi, respectively. FIG. 4B and FIG. 4C show representative images of muscle from wild type and ΔIg3-MuSK mice that were uninjured, 5 dpi, 7 dpi, or 14 dpi, stained either with hematoxylin and eosin (H&E; FIG. 4B) or antibodies to the extracellular matrix protein laminin (green) or Pax7 (red); (FIG. 4C). FIG. 4B and FIG. 4C shows that there was an increased density of Pax7+ satellite cells in ΔIg3-MuSK mice at 5 dpi.

FIG. 5 illustrates characterization of satellite cells (SCs) in uninjured muscle and in regenerating muscle 5, 7, and 14 days post injury (dpi). FIG. 5A shows that ΔIg3-MuSK SCs have significantly increased satellite cell density 5 dpi. SC amounts per cross sectional area were calculated in contralateral control and injured TA muscles 5, 7, 14 dpi, blinded. (n=4 mice, ^{˜}5,000,000 μm counted per mouse, t-test, p=0.02) FIG. 5B shows that ΔIg3-MuSK SCs have significantly increased satellite cell proliferation 5 dpi. Percent of EdU+ SCs was calculated in injured tibialis anterior muscle 5, 7, and 14 dpi, blinded (n=4, t-test, P=0.04). All analysis were done blinded. EdU was administered 24 hours prior to harvest.

FIG. 6 shows the minimum Feret's diameter in wild type and ΔIg3-MuSK muscle collected 7 days after BaCl₂ injury in 5-month old animals. Mann-Whitney T-test: P<0.0001, n=4 mice, 1000-2000 total fibers analyzed per group. The myofiber area is greater at 7 dpi in ΔIg3-MuSK mice compared to wildtype mice, indicating that muscle regeneration is accelerated in ΔIg3-MuSK mice.

FIG. 7 shows generation of a ΔIg3-MuSK mouse model. A mouse model that lacks the MuSK Ig3 domain was generated using CRISPR/Cas9. Plasmids (▴) encoding hSpCas9 and gRNAs flanking the locus that encodes the Ig3 domain were designed to excise an approximate 11 kb region thereby generating a novel MuSK allele (MuSK^{ΔIg3}) that lacks the Ig3 encoding domain. Arrows indicate PCR primers designed to amplify either the WT or MuSK^{ΔIg3}. Founder mice that were homozygous for MuSK^{ΔIg3} were selected by genotyping and confirmed by DNA sequencing. Amplification of genomic DNA of the WT and MuSK^{ΔIg3} alleles by PCR produces amplicons of 436 and 400 bp, respectively. WT mice have the WT MuSK allele but not the MuSK^{ΔIg3}. Heterozygous MuSK^{ΔIg3} mice have both WT and MuSK^{ΔIg3} alleles as evidenced by 436 and 400 bp products, respectively, while MuSK^{ΔIg3} homozygotes only amplify the 436 bp MuSK^{ΔIg3} allele.

FIG. 8. shows histograms of myofiber sizes, as measured by minimum Feret diameter in WT and ΔIg3-MuSK mice. The fiber sizing done on muscle from 3 month old mice shows that there is no difference in average size or size distribution between WT and ΔIg3-MuSK myofibers. The fiber sizing done on 5 month old mice shows ΔIg3-MuSK mice have increased myofiber size. This indicates that ΔIg3-MuSK mice have increased muscle growth between 3 and 5 months. Mann-Whitney T-test: P<0.0001, n=3-4 mice, 500 total fibers analyzed per mouse.

FIG. 9 shows the number of satellite cells in uninjured ΔIg3-MuSK and WT mice at 3 and 5 months. Immunohistochemistry on muscle sections was utilized to determine the number of satellite cells, as measured by Pax7+ cells per area. In three month old animals, the numbers of satellite cells are equivalent between WT and ΔIg3-MuSK muscles. At 5 months, the amount of SCs in WT muscle is unchanged, while the amount of satellite cells in Ig3-MuSK muscle is reduced by about half. Each point represents an animal. This shows that the number of SCs in ΔIg3-MuSK mice declines between 3 and 5 months. Unpaired t-test, n=8 mice, p=0.01.

### DEFINITIONS

For convenience, the meaning of some terms and phrases used in the specification, examples, and appended claims, are provided below. Unless stated otherwise, or implicit from context, the following terms and phrases include the meanings provided below.

These definitions are provided to aid in describing particular embodiments, and are not intended to limit the claimed invention, because the scope of the invention is limited only by the claims.

Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

About or Approximately: The term “about” or “approximately”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system, for example to achieve delivery of an agent (e.g., an agonizing agent) that is, or is included in or otherwise delivered by, the composition. Those of ordinary skill in the art will be aware of a variety of routes that may, in appropriate circumstances, be utilized for administration to a subject, for example a human. For example, in some embodiments, administration may be ocular, oral, buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, an agent (e.g., an agonizing agent) is delivered to the central nervous system (CNS), e.g., delivered via intracerebroventricular administration. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: In general, the term “agent”, as used herein, may be used to refer to a compound or entity of any chemical class including, for example, a polypeptide, nucleic acid, saccharide, lipid, small molecule, metal, or combination or complex thereof. In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Agonist: Those skilled in the art will appreciate that the term “agonist” may be used to refer to an agent (i.e., an “agonizing agent”), condition, or event whose presence, level, degree, type, or form correlates with increased level or activity of another agent (i.e., the agonized agent or the target agent). In general, an agonist may be or include an agent of any chemical class including, for example, small molecules, polypeptides, nucleic acids, carbohydrates, lipids, metals, and/or any other entity that shows the relevant activating activity. In some embodiments, an agonist may be direct (in which case it exerts its influence directly upon its target); in some embodiments, an agonist may be indirect (in which case it exerts its influence by other than binding to its target; e.g., by interacting with a regulator of the target, so that level or activity of the target is altered). In some embodiments, an agonist is a binding agent that is a protein (e.g., an antibody) or a nucleic acid (e.g., an antisense oligonucleotide) that binds a target (e.g., a protein or nucleic acid) so that level, form, and/or or activity of the target is altered. In some embodiments, the altered level, form and/or activity is an increased level of altered protein expressed from the target nucleic acid sequence. Those skilled in the art, reading the present disclosure, will appreciate that, in some embodiments, an agonizing agent may bind to (and potentially agonize) a binding target, which binding causes an increase in level or activity of a further agonized target. To give a specific example, in some embodiments, an agonizing agent that binds to a nucleic acid target may alter level and/or activity of that target, and in some specific embodiments may agonize an activity of that nucleic acid target (e.g., by increasing its modification, splicing, 5′ cap formation, and/or 3′ end formation, transport, and/or translation, etc., so that a level of a desired product—e.g., mRNA, is increased) and/or may agonize a downstream target, such as a polypeptide encoded by such nucleic acid target. To give one particular such example, in some embodiments, an agonizing agent may be or comprise an oligonucleotide that binds to a primary transcript and alters its splicing pattern so that level and/or activity of a particular spliced form (e.g., mature mRNA) is increased, which may, in turn achieved increased level of a product (e.g., a polypeptide) that is or is encoded by such particular spliced form.

Agonist Therapy: The term “agonist therapy”, as used herein, refers to administration of an agonist that agonizes a particular target of interest to achieve a desired therapeutic effect. In some embodiments, agonist therapy involves administering a single dose of an agonist. In some embodiments, agonist therapy involves administering multiple doses of an agonist. In some embodiments, agonist therapy involves administering an agonist according to a dosing regimen known or expected to achieve the therapeutic effect, for example, because such result has been established to a designated degree of statistical confidence, e.g., through administration to a relevant population. In some embodiments, agonist therapy involves delivery of agonizing agent as described herein. As noted above, in some embodiments, an agonizing agent may be or comprise a binding agent that is a protein (e.g., an antibody) or a nucleic acid (e.g., an antisense oligonucleotide) that binds a target (e.g., a protein or nucleic acid) a so that level, form, and/or or activity of the target is altered. In some embodiments, an agonizing agent may bind to (and potentially agonize) a binding target, which binding causes an increase in level or activity of a further agonized target. To give a specific example, in some embodiments, an agonizing agent that binds to a nucleic acid target may alter level and/or activity of that target, and in some specific embodiments may agonize an activity of that nucleic acid target (e.g., by increasing its modification, splicing, 5′ cap formation, and/or 3′ end formation, transport, and/or translation, etc., so that a level of a desired product, e.g., mRNA, is generated) and/or may agonize a downstream target, such as a polypeptide encoded by such nucleic acid target. To give one particular such example, in some embodiment, an agonizing agent may be or comprise an oligonucleotide that binds to a primary transcript and alters its splicing pattern so that level and/or activity of a particular spliced form (e.g., mature mRNA) is generated, which may, in turn achieved increased level of a product (e.g., a polypeptide) that is or is encoded by such particular spliced form.

Antagonist: Those skilled in the art will appreciate that the term “antagonist”, as used herein, may be used to refer to an agent (i.e., an “antagonizing agent”), condition, or event whose presence, level, degree, type, or form correlates with decreased level or activity of another agent (i.e., the inhibited agent, or target). In general, an antagonist may be or include an agent of any chemical class including, for example, small molecules, polypeptides, nucleic acids, carbohydrates, lipids, metals, and/or any other entity that shows the relevant inhibitory activity. In some embodiments, an antagonist may be direct (in which case it exerts its influence directly upon its target); in some embodiments, an antagonist may be indirect (in which case it exerts its influence by other than binding to its target; e.g., by interacting with a regulator of the target, so that level or activity of the target is altered). In some embodiments, an antagonist is binding agent that is a protein (e.g., an antibody) or a nucleic acid (e.g., an antisense oligonucleotide) that binds a target (e.g., a protein or nucleic acid) so that the level, form, and/or activity of the target is altered. In some embodiments, the altered level, form and/or activity is a decreased level of altered protein expressed from the target nucleic acid sequence. Those skilled in the art, reading the present disclosure, will appreciate that, in some embodiments, an antagonizing agent may bind to (and potentially antagonize) a binding target, which binding causes a decrease in level or activity of a further antagonized target. To give a specific example, in some embodiments, an antagonizing agent that binds to a nucleic acid target may alter level and/or activity of that target, and in some specific embodiments may antagonize an activity of that nucleic acid target (e.g., by decreasing its modification, splicing, 5′ cap formation, and/or 3′ end formation, transport, and/or translation, etc., so that a level of an undesired product, e.g., mRNA, is suppressed) and/or may antagonize a downstream target, such as a polypeptide encoded by such nucleic acid target. To give one particular such example, in some embodiment, an antagonizing agent may be or comprise an oligonucleotide that binds to a primary transcript and alters its splicing pattern so that level and/or activity of a particular spliced form (e.g., mature mRNA) is suppressed, which may, in turn achieved decreased level of a product (e.g., a polypeptide) that is or is encoded by such particular spliced form.

Antibody agent: As used herein, the term “antibody agent” refers to an agent that specifically binds to a particular antigen (e.g., that may be or comprise an epitope of a protein of interest—e.g., a MuSK protein). In some embodiments, the term encompasses any polypeptide or polypeptide complex that includes immunoglobulin structural elements sufficient to confer specific binding. Exemplary antibody agents include but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc., as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present invention is in a format selected from, but not limited to, intact IgA, IgG, IgE or IgM antibodies; bi- or multi specific antibodies (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.].

In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments an included CDR is substantially identical to a reference CDR in that it shows at least 96%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments an included CDR is substantially identical to a reference CDR in that at least one amino acid within the included CDR is substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antibody: As used herein, the term “antibody” refers to an immunoglobulin or a derivative thereof containing an immunoglobulin domain capable of binding to an antigen (e.g., that may be or comprise an epitope of a protein of interest, e.g., a MuSK protein). The antibody can be of any species, e.g., human, rodent, rabbit, goat, chicken, etc. The antibody may be a member of any immunoglobulin class, including any of the human classes: IgG, IgM, IgA, IgD, and IgE, or subclasses thereof such as IgG1, IgG2, etc. In various embodiments of the invention the antibody is a fragment such as a Fab′, F(ab′)₂, scFv (single-chain variable) or other fragment that retains an antigen binding site, or a recombinantly produced scFv fragment, including recombinantly produced fragments. See, e.g., Allen (2002) and references therein. The antibody can be monovalent, bivalent or multivalent. The antibody may be a chimeric or “humanized” antibody in which, for example, a variable domain of rodent origin is fused to a constant domain of human origin, thus retaining the specificity of the rodent antibody. The domain of human origin need not originate directly from a human in the sense that it is first synthesized in a human being. Instead, “human” domains may be generated in rodents whose genome incorporates human immunoglobulin genes. See, e.g., Vaughan, et al., (1998). The antibody may be partially or completely humanized. An antibody may be polyclonal or monoclonal, though for purposes of the present invention monoclonal antibodies are generally preferred. Methods for producing antibodies that specifically bind to virtually any molecule of interest are known in the art. For example, monoclonal or polyclonal antibodies can be purified from blood or ascites fluid of an animal that produces the antibody (e.g., following natural exposure to or immunization with the molecule or an antigenic fragment thereof), can be produced using recombinant techniques in cell culture or transgenic organisms, or can be made at least in part by chemical synthesis. In some embodiments, the antibody can act as an antagonist, e.g., by binding to a target antigen, resulting in a decreased level or activity of said antigen. In some embodiments, the antibody can act as an agonist, e.g., by binding to a target antigen, resulting in an increased level or activity of said antigen.

Antisense: The term “antisense” is used herein to refer to a nucleic acid whose nucleotide sequence is complementary to part or all of a sequence found in a coding strand nucleic acid. Typically, a “coding strand” nucleic acid is one whose sequence includes part or all of an open reading frame or other stretch of residues that encodes part or all of a polypeptide. In some embodiments, the term “antisense” may particularly be used herein in reference to an oligonucleotide that binds specifically to a coding strand (i.e., to a target sequence within such coding strand). In some embodiments, a coding strand may include both coding and non-coding sequences (e.g., to give but one example, may be a transcript, such as a primary transcript. that includes both intron and exon sequences). Those skilled in the art, reading the present disclosure, will appreciate that, in some embodiments, an oligonucleotide may be considered or referred to as an “antisense” oligonucleotide when some or all of its sequence is complementary to non-coding portion(s) of its target strand. In some embodiments, an antisense oligonucleotide binds to coding sequences in a target sense strand; in some embodiments, an antisense oligonucleotide binds to non-coding sequences in a target coding strand. In some embodiments, an antisense oligonucleotide binds to both coding and non-coding sequences in a target coding strand. In some embodiments, an antisense oligonucleotide is characterized in that, when bound to its target sequence in a coding strand (e.g., a transcript), it alters post-transcriptional processing (e.g., one or more of modification, splicing, 5′ cap formation, and/or 3′ end formation, 5′ cap formation, and/or 3′ end formation, transport, and/or translation) of such coding strand. In some particular embodiments, an antisense oligonucleotide alters splicing of its target coding strand. Alternatively or additionally, in some embodiments, an antisense-coding strand complex is or can be degraded, e.g., by RNase H.

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Binding agent: In general, the term “binding agent” is used herein to refer to any entity that binds to a target of interest as described herein. In many embodiments, a binding agent of interest is one that binds specifically with its target in that it discriminates its target from other potential binding partners in a particular interaction context. In general, a binding agent may be or comprise an entity of any chemical class (e.g., polymer, non-polymer, small molecule, polypeptide, carbohydrate, lipid, nucleic acid, etc.). In some embodiments, a binding agent is a single chemical entity. In some embodiments, a binding agent is a complex of two or more discrete chemical entities associated with one another under relevant conditions by non-covalent interactions. For example, those skilled in the art will appreciate that in some embodiments, a binding agent may comprise a “generic” binding moiety (e.g., one of biotin/avidin/streptavidin and/or a class-specific antibody) and a “specific” binding moiety (e.g., an antibody or aptamers with a particular molecular target) that is linked to the partner of the generic biding moiety. In some embodiments, such an approach can permit modular assembly of multiple binding agents through linkage of different specific binding moieties with the same generic binding poiety partner. In some embodiments, binding agents are or comprise polypeptides (including, e.g., antibodies or antibody fragments). In some embodiments, binding agents are or comprise small molecules. In some embodiments, binding agents are or comprise nucleic acids (e.g., antisense oligonucleotides). In some embodiments, binding agents are aptamers. In some embodiments, binding agents are polymers; in some embodiments, binding agents are not polymers. In some embodiments, binding agents are non-polymeric in that they lack polymeric moieties. In some embodiments, binding agents are or comprise carbohydrates. In some embodiments, binding agents are or comprise lectins.

In some embodiments, binding agents are or comprise peptidomimetics. In some embodiments, binding agents are or comprise scaffold proteins. In some embodiments, binding agents are or comprise mimeotopes. In some embodiments, binding agents are or comprise stapled peptides. In certain embodiments, binding agents are or comprise nucleic acids, such as DNA or RNA (e.g., antisense oligonucleotides).

Characteristic sequence element: As used herein, the phrase “characteristic sequence element” refers to a sequence element found in a polymer (e.g., in a polypeptide or nucleic acid) that represents a characteristic portion of that polymer. In some embodiments, presence of a characteristic sequence element correlates with presence or level of a particular activity or property of the polymer. In some embodiments, presence (or absence) of a characteristic sequence element defines a particular polymer as a member (or not a member) of a particular family or group of such polymers. A characteristic sequence element typically comprises at least two monomers (e.g., amino acids or nucleotides). In some embodiments, a characteristic sequence element includes at least 2, 3, 4, 5, 6, 7, 8, 9, 10, 11, 12, 13, 14, 15, 20, 25, 30, 35, 40, 45, 50, or more monomers (e.g., contiguously linked monomers). In some embodiments, a characteristic sequence element includes at least first and second stretches of contiguous monomers spaced apart by one or more spacer regions whose length may or may not vary across polymers that share the sequence element.

Complementary: As used herein, in accordance with its art-accepted meaning, “complementary” refers to the capacity for precise pairing between particular bases, nucleosides, nucleotides or nucleic acids. For example, adenine (A) and uridine (U) are complementary; adenine (A) and thymidine (T) are complementary; and guanine (G) and cytosine (C), are complementary and are referred to in the art as Watson-Crick base pairings. If a nucleotide at a certain position of a first nucleic acid sequence is complementary to a nucleotide located opposite in a second nucleic acid sequence when the strands are aligned in anti-parallel orientation, the nucleotides form a complementary base pair, and the nucleic acids are complementary at that position. The percent complementarity of a first nucleic acid to a second nucleic acid may be evaluated by aligning them in antiparallel orientation for maximum complementarity over a window of evaluation, determining the total number of nt in both strands that form complementary base pairs within the window, dividing by the total number of nt within the window, and multiplying by 100. For example, AAAAAAAA and TTTGTTAT are 75% complementary since there are 12 nt in complementary base pairs out of a total of 16 nt. When computing the number of complementary nt needed to achieve a particular % complementarity, fractions are rounded to the nearest whole number. A position occupied by non-complementary nucleotides constitutes a mismatch, i.e., the position is occupied by a non-complementary base pair. In certain embodiments a window of evaluation has the length described herein for duplex portions or target portions. Complementary sequences include base-pairing of a polynucleotide comprising a first nucleotide sequence to a polynucleotide comprising a second nucleotide sequence over the entire length of both nucleotide sequences (if the same length) or over the entire length of the shorter sequence (if different lengths). Such sequences can be referred to as “perfectly complementary” (100% complementarity) with respect to each other herein. Nucleic acids that are at least 70% complementary over a window of evaluation are considered “substantially complementary” over that window. In certain embodiments complementary nucleic acids are at least 75%, at least 80%, at least 85%, at least 90%, or at least 95% complementary over the window of evaluation. Where a first sequence is referred to as “substantially complementary” with respect to a second sequence herein, the two sequences may be perfectly complementary or they may comprise one or more unmatched bases upon hybridization, e.g., up to about 5%, 10%, 15%, 20%, or 25% unmatched bases upon hybridization, e.g., 1, 2, 3, 4, 5, or 6 mismatched base pairs upon hybridization for a duplex up to 30 base pairs, while retaining the ability to hybridize under the conditions most relevant to their intended use. It should be understood that where two oligonucleotides are designed to form, upon hybridization, one or more single stranded overhangs, such overhangs are not regarded as mismatches or unpaired nucleotides with regard to the determination of percent complementarity. For example, the two strands of a dsRNA comprising one oligonucleotide 21 nucleotides in length and another oligonucleotide 23 nucleotides in length, wherein the longer oligonucleotide comprises a sequence of 21 nucleotides that is perfectly complementary to the shorter oligonucleotide and a 2 nucleotide overhang, may be referred to as “perfectly complementary” herein. “Complementary” sequences, as used herein may include one or more non-Watson-Crick base pairs and/or base pairs formed from non-natural and other modified nucleotides, in so far as the requirements with respect to their ability to hybridize are fulfilled. Such non-Watson-Crick base pairs include, but are not limited to, G:U Wobble or Hoogsteen base pairing. Those of ordinary skill in the art are aware that guanine, cytosine, adenine, and uracil can be replaced by other bases without substantially altering the base pairing properties of a polynucleotide comprising a nucleotide bearing such bases, according to the so-called “wobble” rules. See, e.g., Murphy and Ramakrishnan (2004). For example, a nucleotide comprising inosine as its base can base pair with nucleotides containing adenine, cytosine, or uracil. Thus, nucleotides containing uracil, guanine, or adenine can be replaced in the nucleotide sequences of an Inhibitory RNA described herein by a nucleotide containing, for example, inosine. It will be understood that the terms “complementary”, “perfectly complementary”, and “substantially complementary” can be used with respect to the base matching between any two nucleic acids, e.g., the base matching between the sense strand and the antisense strand of a double stranded nucleic acid, or portion thereof. “Hybridize”, as used herein, refers to the interaction between two nucleic acid sequences (which in some embodiments may be part of the same nucleic acid molecule and in other embodiments may be or include part(s) of different nucleic acid molecules) comprising or consisting of complementary portions such that a duplex structure (i.e., an intramolecular or intermolecular duplex) is formed that is stable under the particular conditions of interest, as will be understood by the ordinary skilled artisan.

Comprising: The term “comprising” means that other elements can also be present in addition to the defined elements presented. The use of “comprising” indicates inclusion rather than limitation.

Consisting of: The term “consisting of” refers to compositions, methods, and respective components thereof as described herein, which are exclusive of any element not recited in that description of the embodiment. As used herein the term “consisting essentially of” refers to those elements required for a given embodiment. The term permits the presence of additional elements that do not materially affect the basic and novel or functional characteristic(s) of that embodiment of the invention.

Combination therapy: As used herein, the term “combination therapy” refers to those situations in which a subject is simultaneously exposed to two or more therapeutic regimens (e.g., two or more therapeutic agents). In some embodiments, the two or more regimens may be administered simultaneously; in some embodiments, such regimens may be administered sequentially (e.g., all “doses” of a first regimen are administered prior to administration of any doses of a second regimen); in some embodiments, such agents are administered in overlapping dosing regimens. In some embodiments, “administration” of combination therapy may involve administration of one or more agent(s) or modality(ies) to a subject receiving the other agent(s) or modality(ies) in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Domain: The term “domain” as used herein refers to a section or portion of an entity. In some embodiments, a “domain” is associated with a particular structural and/or functional feature of the entity so that, when the domain is physically separated from the rest of its parent entity, it substantially or entirely retains the particular structural and/or functional feature. Alternatively or additionally, a domain may be or include a portion of an entity that, when separated from that (parent) entity and linked with a different (recipient) entity, substantially retains and/or imparts on the recipient entity one or more structural and/or functional features that characterized it in the parent entity. In some embodiments, a domain is a section or portion of a molecule (e.g., a small molecule, carbohydrate, lipid, nucleic acid, or polypeptide). In some embodiments, a domain is a section of a polypeptide (e.g., the Ig3 domain of a MuSK protein); in some such embodiments, a domain is characterized by a particular structural element (e.g., a particular amino acid sequence or sequence motif, a-helix character, b-sheet character, coiled-coil character, random coil character, etc.), and/or by a particular functional feature (e.g., binding activity, enzymatic activity, folding activity, signaling activity, etc.).

Dosing regimen: Those skilled in the art will appreciate that the term “dosing regimen” may be used to refer to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) transport of an RNA transcript (e.g., from nucleus to cytoplasm; and/or (4) translation of an RNA into a polypeptide or protein; and/or (4) post-translational modification of a polypeptide or protein.

Isolated or Partially Purified: The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated.” The terms “purified” or “substantially purified” refer to an isolated nucleic acid or polypeptide that is at least 95% by weight the subject nucleic acid or polypeptide, including, for example, at least 96%, at least 97%, at least 98%, at least 99% or more. In some embodiments, the antibody, antigen-binding portion thereof, or chimeric antigen receptor (CAR) described herein is isolated. In some embodiments, the antibody, antibody reagent, antigen binding portion thereof, or CAR described herein is purified.

Engineered: As used herein, “engineered” refers to the aspect of having been manipulated by the hand of man. For example, an antibody, antibody reagent, antigen-binding portion thereof, CAR or bispecific antibody is considered to be “engineered” when the sequence of the antibody, antibody reagent, antigen-binding portion thereof, CAR or bispecific antibody is manipulated by the hand of man to differ from the sequence of an antibody as it exists in nature. As is common practice and is understood by those in the art, progeny and copies of an engineered polynucleotide and/or polypeptide are typically still referred to as “engineered” even though the actual manipulation was performed on a prior entity.

Fragment: A “fragment” of a material or entity as described herein has a structure that includes a discrete portion of the whole but lacks one or more moieties found in the whole. In some embodiments, a fragment consists of such a discrete portion. In some embodiments, a fragment consists of or comprises a characteristic structural element or moiety found in the whole. In some embodiments, a polymer fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polymer. In some embodiments, a polymer fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polymer. The whole material or entity may in some embodiments be referred to as the “parent” of the fragment.

Gene: As used herein, the term “gene” refers to a DNA sequence in a chromosome that codes for a product (e.g., an RNA product and/or a polypeptide product). In some embodiments, a gene includes coding sequence (i.e., sequence that encodes a particular product); in some embodiments, a gene includes non-coding sequence. In some particular embodiments, a gene may include both coding (e.g., exonic) and non-coding (e.g., intronic) sequences. In some embodiments, a gene may include one or more regulatory elements that, for example, may control or impact one or more aspects of gene expression (e.g., cell-type-specific expression, inducible expression, etc.).

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene. In some embodiments, a gene product may be or comprise a particular processed form of an RNA transcript (e.g., a particular edited form, a particular splice form, a particular capped form, etc.).

Homology: As used herein, the term “homology” refers to the overall relatedness between polymeric molecules, e.g., between nucleic acid molecules (e.g., DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (1989), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

“Improve, “increase”, “inhibit” or “reduce”: As used herein, the terms “improve”, “increase”, “inhibit’, “reduce”, or grammatical equivalents thereof, indicate values that are relative to a baseline or other reference measurement. In some embodiments, an appropriate reference measurement may be or comprise a measurement in a particular system (e.g., in a single individual, a single cell, or cell population) under otherwise comparable conditions absent presence of (e.g., prior to and/or after) a particular agent or treatment, or in presence of an appropriate reference agent (e.g., a positive control agent or a negative control agent). In some embodiments, an appropriate reference measurement may be or comprise a measurement in comparable system known or expected to respond in a particular way, in presence of the relevant agent or treatment. Those skilled in the art will appreciate that an “improvement”, “increase”, “reduction”, etc. typically refers to a statistically significant change. Moreover, those skilled in the art will understand from context what magnitude of change may be relevant. For example, in some embodiments, a change may be a “fold” change, i.e., so that a “changed” value represents a 1.1, 1.2, 2, 3, 4, 5, 6, 7, 8, 9, 10, 15, 20, 30, 40, 50 or more (e.g., 500, 1000 times) (including all integers and decimal points in between and above 1), e.g., 1.5, 1.6, 1.7. 1.8, etc.)-fold difference relative to the relevant reference. Alternatively or additionally, in some embodiments, a “change” may be a “percentage” change, so that a “changed” value represents a1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% increase or decrease, including all integers and decimal points in between), relative to the relevant reference.

Linked: As used herein, the term “linked”, when used with respect to two or more moieties, means that the moieties are physically associated or connected with one another to form a molecular structure that is sufficiently stable so that the moieties remain associated under the conditions in which the linkage is formed and, preferably, under the conditions in which the new molecular structure is used, e.g., physiological conditions. In certain preferred embodiments of the invention the linkage is a covalent linkage. In other embodiments the linkage is noncovalent. Moieties may be linked either directly or indirectly. When two moieties are directly linked, they are either covalently bonded to one another or are in sufficiently close proximity such that intermolecular forces between the two moieties maintain their association. When two moieties are indirectly linked, they are each linked either covalently or noncovalently to a third moiety, which maintains the association between the two moieties. In general, when two moieties are referred to as being linked by a “linker” or “linking moiety” or “linking portion”, the linkage between the two linked moieties is indirect, and typically each of the linked moieties is covalently bonded to the linker. The linker can be any suitable moiety that reacts with the two moieties to be linked within a reasonable period of time, under conditions consistent with stability of the moieties (which may be protected as appropriate, depending upon the conditions), and in sufficient amount, to produce a reasonable yield.

Internucleotidic linkage: As used herein, the phrase “internucleotidic linkage” refers generally to the phosphorus-containing linkage between nucleotide units of an oligonucleotide and is interchangeable with “inter-sugar linkage” and “phosphorus atom bridge,” as used above and herein. In some embodiments, an internucleotidic linkage is a phosphodiester linkage, as found in naturally occurring DNA and RNA molecules. In some embodiments, an internucleotidic linkage is a “modified internucleotidic linkage” wherein each oxygen atom of the phosphodiester linkage is optionally and independently replaced by an organic or inorganic moiety. In some embodiments, such an organic or inorganic moiety is selected from but not limited to ═S, ═Se, ═NR′, —SR′, —SeR′, —N(R′)₂, B(R′)₃, —S—, —Se—, and —N(R′)—, wherein each R′ is independently as defined and described below. In some embodiments, an internucleotidic linkage is a phosphotriester linkage, phosphorothioate diester linkage

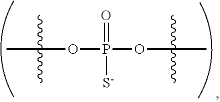

or modified phosphorothioate triester linkage. It is understood by a person of ordinary skill in the art that the internucleotidic linkage may exist as an anion or cation at a given pH due to the existence of acid or base moieties in the linkage. In some embodiments, an internucleotide linkage may be a chiral linkage.

Long-term Administration: As used herein, the term “long-term” administration means that the therapeutic agent or drug is administered for a period of at least 12 weeks. This includes that the therapeutic agent or drug is administered such that it is effective over, or for, a period of at least 12 weeks and does not necessarily imply that the administration itself takes place for 12 weeks, e.g., if sustained release compositions or long acting therapeutic agent or drug is used. Thus, the subject is treated for a period of at least 12 weeks. In many cases, long-term administration is for at least 4, 5, 6, 7, 8, 9 months or more, or for at least 1, 2, 3, 5, 7 or 10 years, or more.

Moiety: Those skilled in the art will appreciate that a “moiety” is a defined chemical group or entity with a particular structure and/or or activity, as described herein.

Nanoparticle: As used herein, the term “nanoparticle” refers to a particle having a diameter of less than 1000 nanometers (nm). In some embodiments, a nanoparticle has a diameter of less than 300 nm, as defined by the National Science Foundation. In some embodiments, a nanoparticle has a diameter of less than 100 nm as defined by the National Institutes of Health. In some embodiments, nanoparticles are micelles in that they comprise an enclosed compartment, separated from the bulk solution by a micellar membrane, typically comprised of amphiphilic entities which surround and enclose a space or compartment (e.g., to define a lumen). In some embodiments, a micellar membrane is comprised of at least one polymer, such as for example a biocompatible and/or biodegradable polymer.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Prodrug: A general, a “prodrug,” as that term is used herein and as is understood in the art, is an entity that, when administered to an organism, is metabolized in the body to deliver an active (e.g., therapeutic or diagnostic) agent of interest. Typically, such metabolism involves removal of at least one “prodrug moiety” so that the active agent is formed. Various forms of “prodrugs” are known in the art.

For examples of such prodrug moieties, see:
a) Design of Prodrugs, edited by H. Bundgaard, (Elsevier, 1985) and Methods in Enzymology, 42:309-396, edited by K. Widder, et al., (Academic Press, 1985);
b) Prodrugs and Targeted Delivery, edited by J. Rautio (Wiley, 2011);
c) A Textbook of Drug Design and Development, edited by Krogsgaard-Larsen;
d) Bundgaard, Chapter 5 “Design and Application of Prodrugs”, by H. Bundgaard, p. 113-191 (1991);
e) Bundgaard, Advanced Drug Delivery Reviews, 8:1-38 (1992);
f) Bundgaard, et al., Journal of Pharmaceutical Sciences, 77:285 (1988); and
g) Kakeya, et al., Chem. Pharm. Bull., 32:692 (1984).

As with other compounds described herein, prodrugs may be provided in any of a variety of forms, e.g., crystal forms, salt forms etc. In some embodiments, prodrugs are provided as pharmaceutically acceptable salts thereof.

Operably linked: As used herein, the term “operably linked” refers to a juxtaposition wherein the components described are in a relationship permitting them to function in their intended manner. A control element “operably linked” to a functional element is associated in such a way that expression and/or activity of the functional element is achieved under conditions compatible with the control element. In some embodiments, “operably linked” control elements (e.g., promoters, enhancers, etc.) are contiguous (e.g., covalently linked) with the coding elements of interest. In some embodiments, control elements act in trans- or cis with the coding functional element of interest.

Patient: As used herein, the term “patient” refers to any organism to which a provided composition (e.g., an agonizing agent such as an ASO) is or may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. In some embodiments, a patient is suffering from or susceptible to one or more disorders or conditions. In some embodiments, a patient displays one or more symptoms of a disorder or condition. In some embodiments, a patient has been diagnosed with one or more disorders or conditions. In some embodiments, the disorder or condition is muscular dystrophy or other disease characterized by neuromuscular dysfunction. In some embodiments, the patient is receiving or has received certain therapy to diagnose and/or to treat a disease, disorder, or condition.

Pharmaceutical composition: As used herein, the term “pharmaceutical composition” refers to an active agent (e.g., an agonizing agent), formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous, intraperitoneal, intrathecal, intravenous, intraventricular or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Pharmaceutically acceptable: As used herein, the phrase “pharmaceutically acceptable” refers to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutically acceptable salt: The term “pharmaceutically acceptable salt”, as used herein, refers to salts of such compounds that are appropriate for use in pharmaceutical contexts, i.e., salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge, et al. (1977) describes pharmaceutically acceptable salts in detail. In some embodiments, pharmaceutically acceptable salt include, but are not limited to, nontoxic acid addition salts, which are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. In some embodiments, pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, a provided compound comprises one or more acidic groups, e.g., an oligonucleotide, and a pharmaceutically acceptable salt is an alkali, alkaline earth metal, or ammonium (e.g., an ammonium salt of N(R)₃, wherein each R is independently defined and described in the present disclosure) salt. Representative alkali or alkaline earth metal salts include sodium, lithium, potassium, calcium, magnesium, and the like. In some embodiments, a pharmaceutically acceptable salt is a sodium salt. In some embodiments, a pharmaceutically acceptable salt is a potassium salt. In some embodiments, a pharmaceutically acceptable salt is a calcium salt. In some embodiments, pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate. In some embodiments, a provided compound comprises more than one acid groups, for example, an oligonucleotide may comprise two or more acidic groups (e.g., in natural phosphate linkages and/or modified internucleotidic linkages). In some embodiments, a pharmaceutically acceptable salt, or generally a salt, of such a compound comprises two or more cations, which can be the same or different. In some embodiments, in a pharmaceutically acceptable salt (or generally, a salt), all ionizable hydrogen (e.g., in an aqueous solution with a pKa no more than about 11, 10, 9, 8, 7, 6, 5, 4, 3, or 2; in some embodiments, no more than about 7; in some embodiments, no more than about 6; in some embodiments, no more than about 5; in some embodiments, no more than about 4; in some embodiments, no more than about 3) in the acidic groups are replaced with cations. In some embodiments, each internucleotidic linkage, e.g., phosphate group, independently exists in its salt form (e.g., if sodium salt, —O—P(O)(ONa)—O—). In some embodiments, a pharmaceutically acceptable salt is a sodium salt of an oligonucleotide. In some embodiments, a pharmaceutically acceptable salt is a sodium salt of an oligonucleotide, wherein each acidic phosphate and modified phosphate group, if any, exists as a salt form (all sodium salt).

Polypeptide: As used herein, the term “polypeptide,” which is interchangeably used herein with the term “protein,” refers to a polymer of at least three amino acid residues. In some embodiments, a polypeptide comprises one or more, or all, natural amino acids. In some embodiments, a polypeptide comprises one or more, or all non-natural amino acids. In some embodiments, a polypeptide comprises one or more, or all, D-amino acids. In some embodiments, a polypeptide comprises one or more, or all, L-amino acids. In some embodiments, a polypeptide comprises one or more pendant groups or other modifications, e.g., modifying or attached to one or more amino acid side chains, at the polypeptide's N-terminus, at the polypeptide's C-terminus, or any combination thereof. In some embodiments, a polypeptide comprises one or more modifications such as acetylation, amidation, aminoethylation, biotinylation, carbamylation, carbonylation, citrullination, deamidation, deimination, eliminylation, glycosylation, lipidation, methylation, pegylation, phosphorylation, sumoylation, or combinations thereof. In some embodiments, a polypeptide may participate in one or more intra- or inter-molecular disulfide bonds. In some embodiments, a polypeptide may be cyclic, and/or may comprise a cyclic portion. In some embodiments, a polypeptide is not cyclic and/or does not comprise any cyclic portion. In some embodiments, a polypeptide is linear. In some embodiments, a polypeptide may comprise a stapled polypeptide. In some embodiments, a polypeptide participates in non-covalent complex formation by non-covalent or covalent association with one or more other polypeptides (e.g., as in an antibody). In some embodiments, a polypeptide has an amino acid sequence that occurs in nature. In some embodiments, a polypeptide has an amino acid sequence that does not occur in nature. In some embodiments, a polypeptide has an amino acid sequence that is engineered in that it is designed and/or produced through action of the hand of man. In some embodiments, the term “polypeptide” may be appended to a name of a reference polypeptide, activity, or structure; in such instances it is used herein to refer to polypeptides that share the relevant activity or structure and thus can be considered to be members of the same class or family of polypeptides. For each such class, the present specification provides and/or those skilled in the art will be aware of exemplary polypeptides within the class whose amino acid sequences and/or functions are known; in some embodiments, such exemplary polypeptides are reference polypeptides for the polypeptide class or family. In some embodiments, a member of a polypeptide class or family shows significant sequence homology or identity with, shares a common sequence motif (e.g., a characteristic sequence element) with, and/or shares a common activity (in some embodiments at a comparable level or within a designated range) with a reference polypeptide of the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (e.g., a conserved region that may in some embodiments comprise a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may comprise a fragment of a parent polypeptide. In some embodiments, a useful polypeptide as may comprise a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide. In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wildtype reference polypeptide's activity according to the assays described below herein. A functional fragment can comprise conservative substitutions of the sequences disclosed herein. In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan. In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

Prevent or prevention: As used herein when used in connection with the occurrence of a disease, disorder, and/or condition, refers to reducing the risk of developing the disease, disorder and/or condition and/or to delaying onset of one or more characteristics or symptoms of the disease, disorder or condition. Prevention may be considered complete when onset of a disease, disorder or condition has been delayed for a predefined period of time.

Recombinant: As used herein, the term “recombinant” is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Small molecule: As used herein, the term “small molecule” means a low molecular weight organic and/or inorganic compound. In general, a “small molecule” is a molecule that is less than about 5 kilodaltons (kD) in size. In some embodiments, a small molecule is less than about 4 kD, 3 kD, about 2 kD, or about 1 kD. In some embodiments, the small molecule is less than about 800 daltons (D), about 600 D, about 500 D, about 400 D, about 300 D, about 200 D, or about 100 D. In some embodiments, a small molecule is less than about 2000 g/mol, less than about 1500 g/mol, less than about 1000 g/mol, less than about 800 g/mol, or less than about 500 g/mol. In some embodiments, a small molecule is not a polymer. In some embodiments, a small molecule does not include a polymeric moiety. In some embodiments, a small molecule is not and/or does not comprise a protein or polypeptide (e.g., is not an oligopeptide or peptide). In some embodiments, a small molecule is not and/or does not comprise a polynucleotide (e.g., is not an oligonucleotide). In some embodiments, a small molecule is not and/or does not comprise a polysaccharide; for example, in some embodiments, a small molecule is not a glycoprotein, proteoglycan, glycolipid, etc.). In some embodiments, a small molecule is not a lipid. In some embodiments, a small molecule is a modulating agent (e.g., is an inhibiting agent or an activating agent). In some embodiments, a small molecule is biologically active. In some embodiments, a small molecule is detectable (e.g., comprises at least one detectable moiety). In some embodiments, a small molecule is a therapeutic agent. Those of ordinary skill in the art, reading the present disclosure, will appreciate that certain small molecule compounds described herein may be provided and/or utilized in any of a variety of forms such as, for example, crystal forms, salt forms, protected forms, pro-drug forms, ester forms, isomeric forms (e.g., optical and/or structural isomers), isotopic forms, etc. Those of skill in the art will appreciate that certain small molecule compounds have structures that can exist in one or more stereoisomeric forms. In some embodiments, such a small molecule may be utilized in accordance with the present disclosure in the form of an individual enantiomer, diastereomer or geometric isomer, or may be in the form of a mixture of stereoisomers. In some embodiments, such a small molecule may be utilized in accordance with the present disclosure in a racemic mixture form. Those of skill in the art will appreciate that certain small molecule compounds have structures that can exist in one or more tautomeric forms. In some embodiments, such a small molecule may be utilized in accordance with the present disclosure in the form of an individual tautomer, or in a form that interconverts between tautomeric forms. Those of skill in the art will appreciate that certain small molecule compounds have structures that permit isotopic substitution (e.g., ²H or ³H for H; ¹¹C, ¹¹C or ¹⁴C for ¹²C; ¹³N or ¹⁵N for 14N; 170 or 180 for 160; ³⁶CI for ³⁵C; ¹⁸F for ¹⁹F; ¹³¹I or ¹²⁵I for ¹²⁷I; etc.). In some embodiments, such a small molecule may be utilized in accordance with the present disclosure in one or more isotopically modified forms, or mixtures thereof. In some embodiments, reference to a particular small molecule compound may relate to a specific form of that compound. In some embodiments, a particular small molecule compound may be provided and/or utilized in a salt form (e.g., in an acid-addition or base-addition salt form, depending on the compound); in some such embodiments, the salt form may be a pharmaceutically acceptable salt form. In some embodiments, where a small molecule compound is one that exists or is found in nature, that compound may be provided and/or utilized in accordance in the present disclosure in a form different from that in which it exists or is found in nature. Those of ordinary skill in the art will appreciate that, in some embodiments, a preparation of a particular small molecule compound that contains an absolute or relative amount of the compound, or of a particular form thereof, that is different from the absolute or relative (with respect to another component of the preparation including, for example, another form of the compound) amount of the compound or form that is present in a reference preparation of interest (e.g., in a primary sample from a source of interest such as a biological or environmental source) is distinct from the compound as it exists in the reference preparation or source. Thus, in some embodiments, for example, a preparation of a single stereoisomer of a small molecule compound may be considered to be a different form of the compound than a racemic mixture of the compound; a particular salt of a small molecule compound may be considered to be a different form from another salt form of the compound; a preparation that contains only a form of the compound that contains one conformational isomer ((Z) or (E)) of a double bond may be considered to be a different form of the compound from one that contains the other conformational isomer ((E) or (Z)) of the double bond; a preparation in which one or more atoms is a different isotope than is present in a reference preparation may be considered to be a different form; etc.

Specific binding: As used herein, the term “specific binding” refers to an ability to discriminate between possible binding partners in the environment in which binding is to occur. A binding agent that interacts with one particular target when other potential targets are present is said to “bind specifically” to the target (e.g., a target amino acid or nucleic acid sequence on a target protein/gene of interest) with which it interacts. In some embodiments, specific binding is assessed by detecting or determining degree of association between the binding agent and its partner; in some embodiments, specific binding is assessed by detecting or determining degree of dissociation of a binding agent-partner complex; in some embodiments, specific binding is assessed by detecting or determining ability of the binding agent to compete an alternative interaction between its partner and another entity. In some embodiments, specific binding is assessed by performing such detections or determinations across a range of concentrations.

Specificity: As is known in the art, “specificity” is a measure of the ability of a particular ligand to distinguish its binding partner from other potential binding partners.

Subject: As used herein, the term “subject” refers an organism, typically a mammal (e.g., a human, in some embodiments including prenatal human forms). In some embodiments, a subject is suffering from a relevant disease, disorder or condition (e.g., muscular dystrophy or other disease characterized by neuromuscular dysfunction). In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantial identity: As used herein refers to a comparison between amino acid or nucleic acid sequences. As will be appreciated by those of ordinary skill in the art, two sequences are generally considered to be “substantially identical” if they contain identical residues in corresponding positions.

As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul et al. (1990 and 1997); Baxevanis et al. (1998); and Misener et al. (1999). In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Suffering from: An individual who is “suffering from” a disease, disorder, and/or condition (e.g., muscular dystrophy or other disease characterized by neuromuscular dysfunction) has been diagnosed with and/or displays one or more symptoms of a disease, disorder, and/or condition.

Susceptible to: An individual who is “susceptible to” a disease, disorder, and/or condition (e.g., muscular dystrophy or other disease characterized by neuromuscular dysfunction) is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Symptoms are reduced: According to the present invention, “symptoms are reduced” when one or more symptoms of a particular disease, disorder or condition (e.g., muscular dystrophy or other disease characterized by neuromuscular dysfunction) is reduced in magnitude (e.g., intensity, severity, etc.) and/or frequency. For purposes of clarity, a delay in the onset of a particular symptom is considered one form of reducing the frequency of that symptom.

Target gene: A “target gene”, as used herein, refers to a gene whose expression is to be modulated, e.g., through modifying splice activity (e.g., by inducing exon-skipping). As used herein, the term “target portion” or “target region” refers to a contiguous portion of the nucleotide sequence of a target gene. In some embodiments, a target portion or target region is one or more exons within the target gene sequence. A target portion may be from about 8-36 nucleotides in length, e.g., about 10-20 or about 15-30 nucleotides in length. A target portion length may have specific value or subrange within the afore-mentioned ranges. For example, in certain embodiments a target portion may be between about 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 nucleotides in length.

Therapeutic agent: As used herein, the phrase “therapeutic agent” refers to any agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition (e.g., one or more symptoms or features of muscular dystrophy or other disease characterized by neuromuscular dysfunction).

Therapeutically effective amount: As used herein, the term “therapeutically effective amount” means an amount of a substance (e.g., a therapeutic agent, composition, and/or formulation) that elicits a desired biological response when administered as part of a therapeutic dosing regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition. As used herein, the terms “effective amount” and “therapeutically-effective amount” include an amount sufficient to prevent or ameliorate a manifestation of disease or medical condition, such as reduced mobility, metabolism, and quality of life resulting from muscle wasting in cancer patients, elderly patients, and many others with no history of neuromuscular dysfunction, in addition to muscular dystrophies such as Becker, Congenital, Distal, Duchenne, Emery-Dreifuss, Facioscapulohumeral, Limb-girdle, Myotonic, Oculopharyngeal Muscular Dystrophy. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds. In some embodiments, a therapeutically effective amount is administered in a single dose; in some embodiments, multiple unit doses are required to deliver a therapeutically effective amount.

Treating: As used herein, the term “treating” refers to providing treatment, i.e., providing any type of medical or surgical management of a subject. The treatment can be provided in order to reverse, alleviate, inhibit the progression of, prevent or reduce the likelihood of a disease, disorder, or condition, or in order to reverse, alleviate, inhibit or prevent the progression of, prevent or reduce the likelihood of one or more symptoms or manifestations of a disease, disorder or condition. Beneficial or desired clinical results include, but are not limited to, alleviation of one or more symptom(s), diminishment of extent of the deficit, stabilized (i.e., not worsening) state of a muscular dystrophy, delay or slowing of muscle wasting, and an increased lifespan as compared to that expected in the absence of treatment. Treating can include administering an agent to the subject following the development of one or more symptoms or manifestations indicative of muscular dystrophy or other disease characterized by neuromuscular dysfunction, e.g., in order to reverse, alleviate, reduce the severity of, and/or inhibit or prevent the progression of the condition and/or to reverse, alleviate, reduce the severity of, and/or inhibit or one or more symptoms or manifestations of the condition. A composition of the disclosure can be administered to a subject who has developed a muscular dystrophy or other disease characterized by neuromuscular dysfunction or is at increased risk of developing such a disorder relative to a member of the general population. A composition of the disclosure can be administered prophylactically, i.e., before development of any symptom or manifestation of the condition. Typically in this case the subject will be at risk of developing the condition.

Variant: As used herein in the context of molecules, e.g., nucleic acids (e.g., ASOs), proteins, or small molecules, the term “variant” refers to a molecule that shows significant structural identity with a reference molecule but differs structurally from the reference molecule, e.g., in the presence or absence or in the level of one or more chemical moieties as compared to the reference entity. In some embodiments, a variant also differs functionally from its reference molecule. In general, whether a particular molecule is properly considered to be a “variant” of a reference molecule is based on its degree of structural identity with the reference molecule. As will be appreciated by those skilled in the art, any biological or chemical reference molecule has certain characteristic structural elements. A variant, by definition, is a distinct molecule that shares one or more such characteristic structural elements but differs in at least one aspect from the reference molecule. To give but a few examples, a polypeptide may have a characteristic sequence element comprised of a plurality of amino acids having designated positions relative to one another in linear or three-dimensional space and/or contributing to a particular structural motif and/or biological function; a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. In some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residues as compared to a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition or deletion, and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

Vector: As used herein, the term “vector” refers to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See, e.g., Sambrook et al., Molecular Cloning: A Laboratory Manual (2d ed.), Cold Spring Harbor Laboratory Press, Cold Spring Harbor, N.Y. (1989).

### DETAILED DESCRIPTION OF CERTAIN EMBODIMENTS

MuSK and Muscle Regeneration and/or Growth

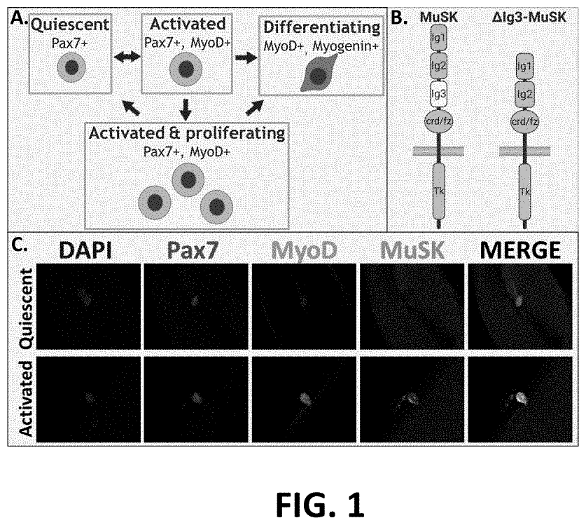

Satellite cells account for about 5% of muscle nuclei and are distributed along mature, multinucleated myofibers, usually in a state of quiescence. When muscle is injured, satellite cells usually proliferate before either returning to quiescence or differentiating (see FIG. 1A). Upon differentiation, satellite cells become committed myoblasts that fuse into myotubes, eventually forming mature myofibers in a process termed myogenesis. Bone morphogenetic protein (BMP) signaling regulates satellite cell dynamics and muscle regeneration both in vivo and in vitro by modulating transcriptional outputs. BMP signaling is not detectable in quiescent satellite cells, it is upregulated in proliferating satellite cells, and it is downregulated during differentiation. However, mediators regulating the balance between satellite cell proliferation and differentiation are unknown.

Muscle-Specific Kinase (MuSK), also known as Muscle-Associated Receptor Tyrosine Kinase, is a transmembrane protein that was first recognized for its essential role in the formation and maintenance of the neuromuscular junction (NMJ). MuSK has three extracellular Immunoglobulin (Ig)-like domains and a cysteine-rich frizzled (CRD/Fz) domain, as well as an intracellular tyrosine kinase (TK) domain. The Ig1, TK and potentially the CRD/Fz domains are required for NMJ formation and maintenance. The Ig1 and TK domains are essential for agrin-LRP4 signaling directing synaptic differentiation. For this reason, global deletion MuSK mice are neo-natal lethal. Two isoforms of MuSK that exist in vivo are: full length (FL) MuSK and a naturally-occurring splice variant that lacks the Ig3 domain (ΔIg3-MuSK) (FIG. 1B). FL MuSK mRNA levels are 10× higher than mRNA of ΔIg3-MuSK but the two are expressed coordinately.

Exemplary amino acid sequences of human and mouse MuSK Ig3 domains (i.e., of MuSK Ig3 domain polypeptides) are as set out below:

MuSK HUMAN_Ig3_Domain:
(SEQ ID NO: 1)
ARILRAPESHNVTFGSFVTLHCTATGIPVPTITWIENGNAVSSGSIQESV
KDRVIDSRLQLFITKPGLYTCIATNKHGEKFSTAKAAATIS
MuSK MOUSE Ig3 Domain
(SEQ ID NO: 2)
ARILRAPESHNVTFGSFVTLRCTAIGIPVPTISWIENGNAVSSGSIQESV
KDRVIDSRLQLFITKPGLYTCIATNKHGEKFSTAKAAATVS

Among other things, the present disclosure teaches that regulating MuSK alternative splicing is a strategy for increasing muscle regeneration.

MuSK is activated by a nerve-derived proteoglycan called agrin. Agrin has been characterized for its role in the development of the neuromuscular junction during embryogenesis. Agrin is named based on its involvement in the aggregation of acetylcholine receptors during synaptogenesis. In humans, this protein is encoded by the AGRN gene. The agrin protein has nine domains homologous to protease inhibitors.

MuSK is expressed in muscle and is upregulated during muscle regeneration. Data suggest that MuSK is implicated in BMP signaling in myogenesis. MuSK can act as a BMP co-receptor that binds BMP2, BMP4, and BMP7 as well as the Type I BMP receptors ALK3 and ALK6. See, e.g., Yilmaz et al. (2016). The Ig3 domain of MuSK is required for high-affinity binding to BMP. MuSK upregulates BMP signaling as measured by BMP4-dependent phosphorylation of SMAD1/5/8. Importantly, MuSK-BMP signaling shapes the magnitude and composition of BMP-induced transcriptome in myoblasts and myotubes and this role is independent of any MuSK tyrosine kinase activity. MuSK is a BMP co-receptor that potentiates BMP signaling and regulates myogenic factors, such as myogenic factor 5 (Myf5), in immortalized myogenic cells.

As described herein, activated satellite cells express MuSK protein and disruption of MuSK-BMP signaling alters satellite cell proliferation in regenerating muscle in vivo. Data included herein provide information on the role of the MuSK-BMP pathway in satellite cells and muscle regeneration. These data suggest that the MuSK-BMP pathway plays a key role, and the present disclosure provides an insight that targeting the MuSK-BMP pathway is more selective than targeting BMP-wide activity, at least because MuSK expression is far more restricted than that of the BMP system. Data provided herein also teach that targeting MuSK-BMP pathway enhances muscle growth. In some embodiments, muscle growth occurs in, e.g., uninjured tissues.

### MuSK Muscle Regeneration (MR) Agonizing Agents

In some embodiments, the present disclosure provides technologies for achieving (e.g., inducing, enhancing, etc.) muscle regeneration in a subject by administering an agent (i.e., a MuSK muscle regeneration (MuSK MR) agonizing agent) in whose presence MuSK muscle regeneration level and/or activity in increased. As described herein, muscle regeneration (MR) may also include or contribute to increased muscle growth. For example, in some embodiments, a MuSK MR agonizing agent is an agent that increases level or activity of one or more MuSK polypeptides (e.g., ΔIg3-MuSK) that lack(s) an effective Ig3 domain, e.g., because such domain has been mutated, removed, or otherwise inactivated (e.g., by blocking, modification, etc.). Alternatively or additionally, in some embodiments, a MuSK MR agonizing agent is one that blocks, inactivates, mutates, or removes a functional Ig3 domain from MuSK, or achieves, supports, or contributes to such blocking, inactivation, mutation or removal.

In some embodiments, the present disclosure provides technologies that relate to MuSK MR agonizing agents—including, for example, providing such agents themselves, and/or providing methods and/or reagents for identifying, characterizing, manufacturing and/or using them and/or compositions that comprise and/or deliver them.

In some embodiments, a MuSK MR agonizing agent may interact directly with a MuSK polypeptide (e.g., with full-length MuSK and/or with ΔIg3-MuSK). In some embodiments, a MuSK MR agonizing agent may not interact directly with a MuSK polypeptide but rather, through some other interaction (e.g., with a precursor or regulator or downstream product of MuSK), impacts level and/or activity of ΔIg3-MuSK.

In principle, an ΔIg3-MuSK MR agonizing agent may be of any chemical class (e.g., small molecule, polypeptide [e.g., antibody], nucleic acid, etc.). In some embodiments, a MuSK MR agonizing agent is agent that downregulates the MuSK Ig3 domain protein expression, the MuSK Ig3 domain gene expression, and/or the MuSK Ig3 activation of BMP signaling, thereby inducing muscle regeneration. In some embodiments, a MuSK MR agonizing agent is an agonizing agent that increases expression of MuSK ΔIg3. In some particular embodiments, described in more detail herein, a MuSK MR agonizing agent may be or comprise a small molecule. In some particular embodiments, described in more detail herein, a MuSK MR agonizing agent may be or comprise an antibody that binds to a MuSK polypeptide (e.g., an antibody that blocks MuSK Ig3 and/or that sequesters one or more MuSK polypeptide forms that include functional Ig3). In some particular embodiments, described in more detail herein, a MuSK MR agonizing agent may be or comprise a nucleic acid agent. For example, in some embodiments, a MuSK MR agonizing agent may be or comprise a nucleic acid (e.g., a gene therapy vector or an RNA therapeutic such as an mRNA) encoding a MuSK form (e.g., ΔIg3-MuSK) that lacks a functional Ig3 domain. Alternatively or additionally, in some embodiments, a nucleic acid MuSK MR agonizing agent may be or comprise an oligonucleotide, such as a MuSK Ig3-targeted exon-skipping oligonucleotide, a MuSK Ig3-targeted CRISPR/Cas9 gRNA (e.g., that modifies and/or removes Ig3), a MuSK Ig3-targeted siRNA (e.g., that inhibits production/expression of MuSK Ig3, for example from a transcript that encodes it), and/or a MuSK Ig3-targeted shRNA.

Small Molecules

In some embodiments, a MuSK MR agonizing agent may be or comprise a small molecule compound.

In some embodiments, a small molecule MuSK MR agonizing agent targets therapeutic agent that targets MuSK splicing; for example, in some embodiments such a small molecule compound enhances splicing event(s) that generate(s) message encoding ΔIg3-MuSK and/or inhibits splicing event(s) that generate(s) message(s) encoding other MuSK splice variants. In some embodiments, a small molecule MuSK MR agonizing agent alters the BMP signaling pathway. In some embodiments, a small molecule MuSK MR agonizing agent alters the BMP signaling pathway which further induces muscle regeneration.

In some embodiments, a small molecule MuSK MR agonizing agent targets one or more of type I BMP receptors, ALK3 (ALK is Anaplastic lymphoma kinase) and ALK6, and type I activin receptor ALK4. In some embodiments, a small molecule MuSK MR agonizing agent is an ALK inhibitor. In some embodiments, a small molecule MuSK MR agonizing agent is an ALK inhibitor selected from the group consisting of crizotinib, ceritinib, alectinib, brigatinib, lorlatinib.

In some embodiments, a small molecule MuSK MR agonizing agent targets the MuSK Ig3 domain and/or BMP so that level and/or activity of a MuSK/BMP complex is reduced. In some such embodiments, a small molecule MuSK MR agonizing agent inhibits formation of and/or disrupts such complex(es). In some embodiments, such a MuSK MR agonizing agent competes with BMP for binding to MuSK Ig3 and/or competes with MuSK Ig3 for binding to BMP.

Antibody Agents

In some embodiments, a MuSK MR agonizing agent is an antibody agent.

In some embodiments, such an antibody agent specifically binds to a MuSK polypeptide. In some embodiments, an antibody agent targeting MuSK specifically binds to the Ig3 domain of a MuSK polypeptide.

In some embodiments, an antibody targeting the Ig3 domain of MuSK protein may bind specifically to the Ig3 domain relative to the Ig1 or Ig2 domains of MuSK.

In some embodiments, an anti-MuSK antibody agent targets the MuSK Ig3 domain and/or BMP so that level and/or activity of a MuSK/BMP complex is reduced. In some such embodiments, an anti-MuSK antibody agent inhibits formation of and/or disrupts such complex(es). In some embodiments, such an anti-MuSK antibody agent competes with BMP for binding to MuSK Ig3 and/or competes with MuSK Ig3 for binding to BMP.

In some embodiments, an anti-MUSK antibody agent is internalized by a cell (e.g., a satellite cell). In some embodiments, an antibody agent (e.g., that is or comprises an antibody or antigen-binding fragment thereof) described herein can be or comprise an immunoglobulin, heavy chain antibody, light chain antibody, or other protein scaffold with antibody-like properties, as well as other immunological binding moiety known in the art, including a Fab fragment, a Fab′ fragment, a F(ab′)2 fragment, a Fv fragment, a disulfide-bonded Fv fragment, a scFv fragment, a diabody, a triabody, a tetrabody, a minibody, a maxibody, a tandab, BiTe, and any combination thereof.

In some embodiments, anti-MUSK antibody agents target, for example, the Ig3 domain of MUSK. In some embodiments, such antibody agents, may inhibit or substantially prevent the binding of a BMP to the MuSK Ig3 domain.

In some embodiments an antibody agent may be or comprise an antibody which, for example, can be an immunoglobulin molecule of four polypeptide chains, e.g., two heavy (H) chains and two light (L) chains. A heavy chain can include a heavy chain variable domain and a heavy chain constant domain. A heavy chain constant domain can include CH1, hinge, CH2, CH3, and in some instances CH4 regions. A light chain can include a light chain variable domain and a light chain constant domain. A light chain constant domain can include a CL. A heavy chain variable domain of a heavy chain and a light chain variable domain of a light chain can typically be further subdivided into regions of variability, termed complementarity determining regions (CDRs), interspersed with regions that are more conserved, termed framework regions (FR). Such heavy chain and light chain variable domains can each include three CDRs and four framework regions, arranged from amino-terminus to carboxyl-terminus in the following order: FR1, CDR1, FR2, CDR2, FR3, CDR3, FR4, one or more of which can be engineered as described herein.

In some embodiments, an antibody agent (e.g., anti-MUSK antibodies) can include various heavy chains and light chains described herein. In some embodiments, an antibody can include two heavy chains and light chains. In various embodiments, the present disclosure encompasses an antibody including at least one heavy chain and/or light chain as disclosed herein, at least one heavy chain and/or light chain framework domain as disclosed herein, at least one heavy chain and/or light chain CDR domain as disclosed herein, and/or any heavy chain and/or light chain constant domain as disclosed herein.

In some embodiments, an antibody agent is or comprises a monoclonal antibody. Typically, monoclonal antibodies ae obtained from a population of substantially homogeneous an antibodies, i.e., the individual antibodies comprising the population are substantially identical except for possible naturally occurring mutations that may be present in minor amounts. Thus, the modifier “monoclonal” as used herein, indicates the character of the antibody as not being a mixture of discrete antibodies. In some embodiments, monoclonal antibodies directed to a particular epitope are derived from a single cell line (e.g., a B cell line).

In some embodiments, an antibody agent (e.g., an anti-MUSK antibody) may be or comprise a polyclonal antibody. In contrast to monoclonal antibodies, polyclonal antibodies typically represent a population of heterogeneous antibodies, i.e., the antibodies in a particular population include structural variation, for example, affinity for different epitope(s) on a particular antigen (e.g., the Ig3 domain of MuSK, or a region within the Ig3 domain). Several methods of producing polyclonal antibodies are known in the art, including use of multiple subcutaneous and/or intraperitoneal injections of the relevant antigen into an animal, optionally including co-administration of one or more adjuvants.

Oligonucleotides

In some embodiments, a MuSK MR agonizing agent as described herein is or comprises an oligonucleotide.

Synthetic oligonucleotides provide useful molecular tools in a wide variety of applications. For example, oligonucleotides are useful in therapeutic, diagnostic, research, and new nanomaterials applications. The use of naturally occurring nucleic acids (e.g., unmodified DNA or RNA) is limited, for example, by their susceptibility to endo- and exo-nucleases. As such, various synthetic counterparts have been developed to circumvent these shortcomings. These include synthetic oligonucleotides that contain chemical modification, e.g., base modifications, sugar modifications, backbone modifications, etc., which, among other things, render these molecules less susceptible to degradation and improve other properties of oligonucleotides. Chemical modifications may also lead to certain undesired effects, such as increased toxicities, etc.

Among other things, the present disclosure encompasses the recognition that structural elements of oligonucleotides, such as base sequence, chemical modifications (e.g., modifications of sugar, base, and/or internucleotidic linkages, and patterns thereof), and/or stereochemistry (e.g., stereochemistry of backbone chiral centers (chiral internucleotidic linkages), and/or patterns thereof), can have significant impact on properties, e.g., stability, splicing-altering capabilities, etc. In some embodiments, oligonucleotide properties can be adjusted by optimizing chemical modifications (modifications of base, sugar, and/or internucleotidic linkage) and/or stereochemistry (pattern of backbone chiral centers).

In some embodiments, the present disclosure demonstrates that oligonucleotide compositions comprising oligonucleotides with controlled structural elements, e.g., controlled chemical modification, provide unexpected properties, including but not limited to those described herein. In some embodiments, provided compositions comprising oligonucleotides having chemical modifications (e.g., base modifications, sugar modification, internucleotidic linkage modifications, etc.) have improved properties, such as improved splicing-altering capabilities, or improved protein binding profile, and/or improved delivery, etc. Particularly, in some embodiments, the present disclosure provides compositions and methods for altering splicing of transcripts. In some embodiments, the present disclosure provides compositions and methods for improving splicing of transcripts. In some embodiments, altered transcript splicing by provided compositions and methods include production of products having desired and/or improved biological functions, and/or knockdown of undesired product by, e.g., modifying splicing products so that undesired biological functions can be suppressed or removed.

In some embodiments, a splicing product is mRNA. In some embodiments, alteration comprises skipping one or more exons. In some embodiments, splicing of a transcript is improved in that exon skipping increases levels of mRNA and proteins that have improved beneficial activities compared with absence of exon skipping.

In some embodiments, splicing of a transcript is improved in that exon skipping lowers levels of mRNA and proteins that have undesired activities compared with absence of exon skipping. In some embodiments, a target is knocked down through exon skipping which, by skipping one or more exons, causes premature stop codon and/or frameshift mutations.

In some embodiments, an oligonucleotide of the disclosure includes one or more natural nucleobase and/or one or more modified nucleobases derived from a natural nucleobase. Examples include, but are not limited to, uracil, thymine, adenine, cytosine, and guanine having their respective amino groups protected by acyl protecting groups, 2-fluorouracil, 2-fluorocytosine, 5-bromouracil, 5-iodouracil, 2,6-diaminopurine, azacytosine, pyrimidine analogs such as pseudoisocytosine and pseudouracil and other modified nucleobases such as 8-substituted purines, xanthine, or hypoxanthine (the latter two being the natural degradation products).

Modified nucleobases also include expanded-size nucleobases in which one or more aryl rings, such as phenyl rings, have been added.

In some embodiments, modified nucleobases are of any one of the following structures, optionally substituted:

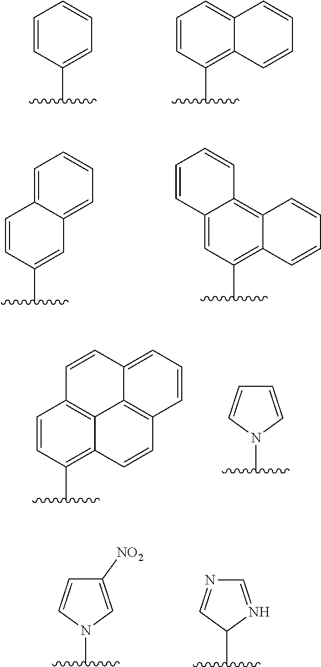

In some embodiments, a modified nucleobase is unsubstituted. In some embodiments, a modified nucleobase is substituted. In some embodiments, a modified nucleobase is substituted such that it contains, e.g., heteroatoms, alkyl groups, or linking moieties connected to fluorescent moieties, biotin or avidin moieties, or other protein or peptides. In some embodiments, a modified nucleobase is a “universal base” that is not a nucleobase in the most classical sense, but that functions similarly to a nucleobase. One representative example of such a universal base is 3-nitropyrrole.

In some embodiments, an oligonucleotide described herein includes nucleosides that incorporate modified nucleobases and/or nucleobases covalently bound to modified sugars. Some examples of nucleosides that incorporate modified nucleobases include 4-acetylcytidine; 5-(carboxyhydroxylmethyl)uridine; 2′-O-methylcytidine; 5-carboxymethylaminomethyl-2-thiouridine; 5-carboxymethylaminomethyluridine; dihydrouridine; 2′-O-methylpseudouridine; beta,D-galactosylqueosine; 2′-O-methylguanosine; N⁶-isopentenyladenosine; 1-methyladenosine; 1-methylpseudouridine; 1-methylguanosine; I-methylinosine; 2,2-dimethylguanosine; 2-methyladenosine; 2-methylguanosine; N⁷-methylguanosine; 3-methyl-cytidine; 5-methylcytidine; 5-hydroxymethylcytidine; 5-methylcytosine, 5-formylcytosine; 5-carboxylcytosine; N⁶-methyladenosine; 7-methylguanosine; 5-methylaminoethyluridine; 5-methoxyaminomethyl-2-thiouridine; beta,D-mannosylqueosine; 5-methoxycarbonylmethyluridine; 5-methoxyuridine; 2-methylthio-N⁶-isopentenyladenosine; N-((9-beta,D-ribofuranosyl-2-methylthiopurine-6-yl)carbamoyl)threonine; N-((9-beta,D-ribofuranosylpurine-6-yl)-N-methylcarbamoyl)threonine; uridine-5-oxyacetic acid methylester; uridine-5-oxyacetic acid (v); pseudouridine; queosine; 2-thiocytidine; 5-methyl-2-thiouridine; 2-thiouridine; 4-thiouridine; 5-methyluridine; 2′-O-methyl-5-methyluridine; and 2′-O-methyluridine.

In some embodiments, nucleosides include 6′-modified bicyclic nucleoside analogs that have either (R) or (S)-chirality at the 6′-position and include the analogs described in U.S. Pat. No. 7,399,845. In other embodiments, nucleosides include 5′-modified bicyclic nucleoside analogs that have either (R) or (S)-chirality at the 5′-position and include the analogs described in U.S. Publ. No. 20070287831. In some embodiments, a nucleobase or modified nucleobase is 5-bromouracil, 5-iodouracil, or 2,6-diaminopurine. In some embodiments, a nucleobase or modified nucleobase is modified by substitution with a fluorescent moiety.

In some embodiments, an oligonucleotide described herein includes one or more modified nucleotides wherein a phosphate group or linkage phosphorus in the nucleotides are linked to various positions of a sugar or modified sugar. As non-limiting examples, the phosphate group or linkage phosphorus can be linked to the 2′, 3′, 4′ or 5′ hydroxyl moiety of a sugar or modified sugar. Nucleotides that incorporate modified nucleobases as described herein are also contemplated in this context.

Other modified sugars can also be incorporated within an oligonucleotide molecule. In some embodiments, a modified sugar contains one or more substituents at the 2′ position including one of the following: —F; —CF₃, —CN, —N₃, —NO, —NO₂, —OR′, —SR′, or —N(R′)₂, wherein each R′ is independently as defined above and described herein; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein the alkyl, alkylene, alkenyl and alkynyl may be substituted or unsubstituted. Examples of substituents include, and are not limited to, —O(CH₂)ₙOCH₃, and —O(CH₂)ₙNH₂, wherein n is from 1 to about 10, MOE, DMAOE, DMAEOE.

In some embodiments, the 2′-OH of a ribose is replaced with a substituent including one of the following: —H, —F; —CF₃, —CN, —N₃, —NO, —NO₂, —OR′, —SR′, or —N(R′)₂, wherein each R′ is independently as defined above and described herein; —O—(C₁-C₁₀ alkyl), —S—(C₁-C₁₀ alkyl), —NH—(C₁-C₁₀ alkyl), or —N(C₁-C₁₀ alkyl)₂; —O—(C₂-C₁₀ alkenyl), —S—(C₂-C₁₀ alkenyl), —NH—(C₂-C₁₀ alkenyl), or —N(C₂-C₁₀ alkenyl)₂; —O—(C₂-C₁₀ alkynyl), —S—(C₂-C₁₀ alkynyl), —NH—(C₂-C₁₀ alkynyl), or —N(C₂-C₁₀ alkynyl)₂; or —O—(C₁-C₁₀ alkylene)-O—(C₁-C₁₀ alkyl), —O—(C₁-C₁₀ alkylene)-NH—(C₁-C₁₀ alkyl) or —O—(C₁-C₁₀ alkylene)-NH(C₁-C₁₀ alkyl)₂, —NH—(C₁-C₁₀ alkylene)-O—(C₁-C₁₀ alkyl), or —N(C₁-C₁₀ alkyl)-(C₁-C₁₀ alkylene)-O—(C₁-C₁₀ alkyl), wherein the alkyl, alkylene, alkenyl and alkynyl may be substituted or unsubstituted. In some embodiments, the 2′-OH is replaced with —H (deoxyribose). In some embodiments, the 2′-OH is replaced with —F. In some embodiments, the 2′-OH is replaced with —OR′. In some embodiments, the 2′-OH is replaced with —OMe. In some embodiments, the 2′-OH is replaced with —OCH₂CH₂OMe (MOE).

Modified sugars also include locked nucleic acids (LNAs). In some embodiments, the locked nucleic acid has the structure indicated below. A locked nucleic acid of the structure below is indicated, wherein Ba represents a nucleobase or modified nucleobase as described herein, and wherein R^{2s} is —OCH₂C₄′-

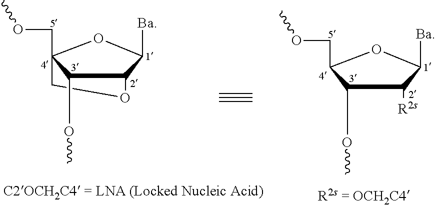

In some embodiments, the present invention provides an oligonucleotide comprising one or more modified internucleotidic linkages independently having the structure of formula I:

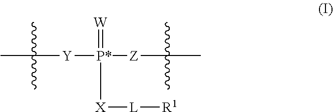

wherein:
P* is an asymmetric phosphorus atom and is either Rp or Sp;
W is O, S or Se;
each of X, Y and Z is independently —O—, —S—, —N(-L-R¹)—, or L;
L is a covalent bond or an optionally substituted, linear or branched C₁-C₁₀ alkylene, wherein one or more methylene units of L are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
R¹ is halogen, R, or an optionally substituted C₁-C₅₀ aliphatic wherein one or more methylene units are optionally and independently replaced by an optionally substituted C₁-C₆ alkylene, C₁-C₆ alkenylene, —C≡C—, —C(R′)₂—, -Cy-, —O—, —S—, —S—S—, —N(R′)—, —C(O)—, —C(S)—, —C(NR′)—, —C(O)N(R′)—, —N(R′)C(O)N(R′)—, —N(R′)C(O)—, —N(R′)C(O)O—, —OC(O)N(R′)—, —S(O)—, —S(O)₂—, —S(O)₂N(R′)—, —N(R′)S(O)₂—, —SC(O)—, —C(O)S—, —OC(O)—, or —C(O)O—;
each R′ is independently —R, —C(O)R, —CO₂R, or —SO₂R, or:
   two R′ on the same nitrogen are taken together with their intervening atoms to form an optionally substituted heterocyclic or heteroaryl ring, or
   two R′ on the same carbon are taken together with their intervening atoms to form an optionally substituted aryl, carbocyclic, heterocyclic, or heteroaryl ring;
-Cy- is an optionally substituted bivalent ring selected from phenylene, carbocyclylene, arylene, heteroarylene, or heterocyclylene;
each R is independently hydrogen, or an optionally substituted group selected from C₁-C₆ aliphatic, phenyl, carbocyclyl, aryl, heteroaryl, or heterocyclyl; and
each

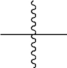

independently represents a connection to a nucleoside.

In some embodiments, the internucleotidic linkage having the structure of formula I is

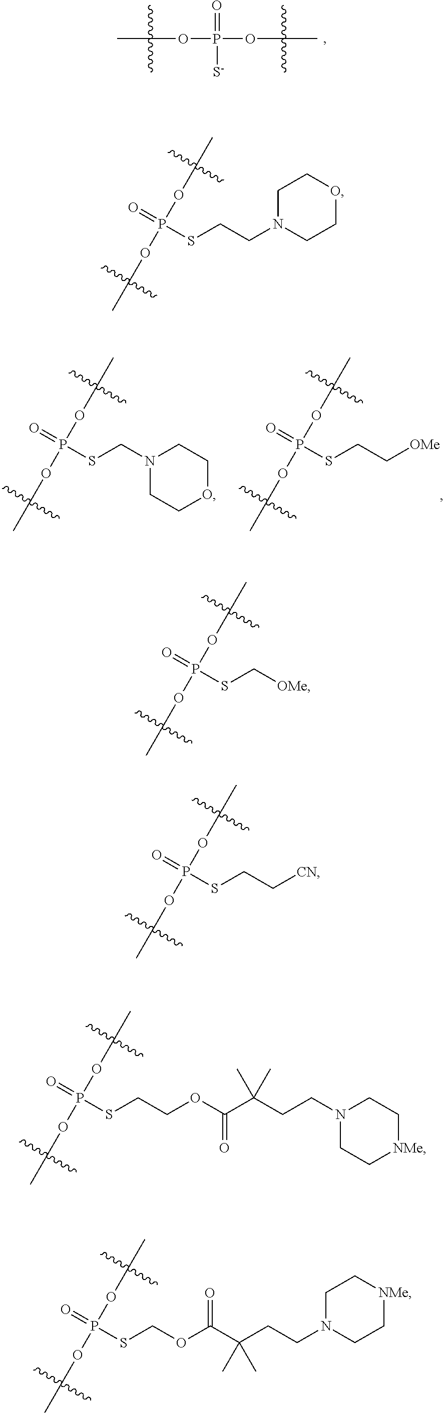

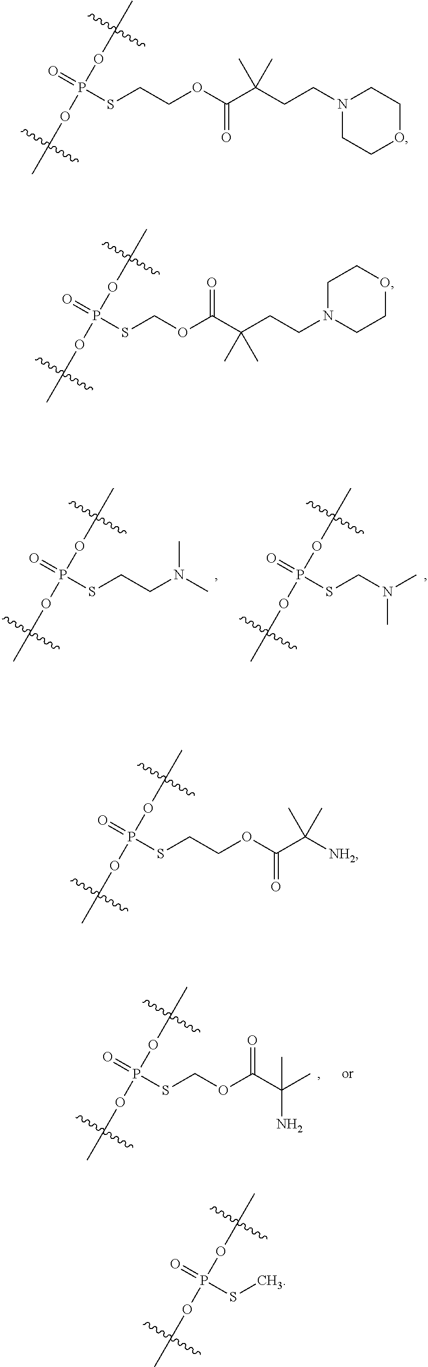

Among other things, the present disclosure provides oligonucleotides of various designs, which may comprise various nucleobases and patterns thereof, sugars and patterns thereof, internucleotidic linkages and patterns thereof, and/or additional chemical moieties and patterns thereof as described in the present disclosure. In some embodiments, provided oligonucleotides can downregulates the MuSK Ig3 domain protein expression, the MuSK Ig3 domain gene expression, and/or the MuSK Ig3 activation of BMP signaling level, thereby increasing muscle regeneration. In some embodiments, provided oligonucleotides can direct a decrease in the expression, level and/or activity of MuSK Ig3 domain and/or one or more of its products in a cell of a subject or patient. In some embodiments, a cell normally expresses or produces protein encoded by MuSK Ig3 domain. In some embodiments, provided oligonucleotides can direct a decrease in the expression, level and/or activity of MuSK Ig3 domain gene or a gene product and has a base sequence which consists of, comprises, or comprises a portion (e.g., a span of 10, 11, 12, 13, 14, 15, 16, 17, 18, 19, 20 or more contiguous bases) of the base sequence of a oligonucleotide disclosed herein, wherein each T can be independently substituted with U and vice versa, and the oligonucleotide comprises at least one non-naturally-occurring modification of a base, sugar and/or internucleotidic linkage.

As described herein, the highly abundant full length MuSK harbors the BMP-binding Ig3 domain and potentiates BMP signaling and affects muscle regeneration. In contrast, ΔIg3-MuSK has lower BMP signaling and promotes muscle regeneration and/or prevents muscle fibrosis. In some embodiments, the present disclosure provides exon-skipping ASOs that switch MuSK from the full length MuSK to the muscle-promoting ΔIg3-MuSK splice form.

In some embodiments, one or more skipped exons are selected from exon 6 and 7 or MuSK gene. In some embodiments, exon 6 of MuSK is skipped. In some embodiments, exon 7 of MuSK is skipped. In some embodiments, both exons 6 and 7 of MuSK are skipped.

In various embodiments, an active compound is an oligonucleotide that directs skipping of one or more exons in a MuSK gene. In various embodiments, an active compound is an oligonucleotide that directs skipping of multiple exons in a MuSK gene. In some embodiments, an active compound is an oligonucleotide that directs skipping of exon 6, exon 7, or both in a MuSK gene. In some embodiments, an active compound is an oligonucleotide that directs skipping of exon 6 in a MuSK gene. In some embodiments, an active compound is an oligonucleotide that directs skipping of exon 7 in a MuSK gene. In some embodiments, an active compound is an oligonucleotide that directs skipping of exons 6 and 7 in a MuSK gene. In some embodiments, a plurality of oligonucleotides may be used together. In some such embodiments, two or more different exon skipping oligonucleotides (e.g., at least one that directs skipping of exon 6 and one that directs skipping of exon 7) may be used in combination. Alternatively or additionally, in some embodiments, at least one exon skipping oligonucleotide may be used in combination with at least one degrading oligonucleotide (e.g., that targets a transcript for RNase H degradation) which, for example, may target MuSK transcript(s) that include a functional Ig3 domain, or portion thereof.

In some embodiments, oligonucleotides are provided and/or utilized in salt forms. In some embodiments, oligonucleotides are provided as salts comprising negatively-charged internucleotidic linkages (e.g., phosphorothioate internucleotidic linkages, natural phosphate linkages, etc.) existing as their salt forms. In some embodiments, oligonucleotides are provided as pharmaceutically acceptable salts. In some embodiments, oligonucleotides are provided as metal salts. In some embodiments, oligonucleotides are provided as sodium salts. In some embodiments, oligonucleotides are provided as metal salts, e.g., sodium salts, wherein each negatively-charged internucleotidic linkage is independently in a salt form (e.g., for sodium salts, —O—P(O)(SNa)—O— for a phosphorothioate internucleotidic linkage, —O—P(O)(ONa)—O— for a natural phosphate linkage, etc.).

### Characterization of MuSK MR Agonizing Agents

MuSK muscle regeneration (MR) agonizing agents provided herein may be identified, assessed and/or characterized for one or more their physical/chemical properties and/or biological activities. Those skilled in the art will be aware of a variety of approaches, including particular assays, that may be utilized for such identification, assessment, and/or characterization.

In some embodiments, a small molecule MuSK MR agonizing agent may interfere with interaction between MuSK Ig3 and BMP, for example by binding directly to MuSK Ig3 or to BMP. In some such embodiments, such agents may be characterized by direct binding assays (e.g., that assess their affinity for, specificity to, and/or one or more kinetic or thermodynamic features of their interaction with, their target(s)) to MuSK Ig3 and/or to BMP, and/or by competitive binding assays (e.g., that assess their ability to disrupt or undue pre-formed complexes of MuSK Ig3 and BMP and/or to reduce complex formation). In some embodiments, such biding assays are desirably performed at multiple concentrations; in some embodiments, such binding assays may be performed with full-length MuSK, or with some other polypeptide or agent that is or comprises MuSK Ig3.

In some embodiments, a MuSK MR agonizing agent, (e.g., an antibody or small molecule that binds the Ig3 domain of MuSK), when contacted with a cell expressing MuSK, will compete with BMP for binding of the Ig3 domain. In some embodiments, such an antibody agent specifically binds to an epitope of MuSK that is expressed in a particular cell type (e.g., a satellite cell). In some embodiments, such an antibody agent may have a binding affinity (e.g., as measured by a dissociation constant) for MuSK protein, e.g., the Ig3 domain of MuSK protein) of at least about 10⁻⁴ M, at least about 10⁻⁵ M, at least about 10⁻⁶ M, at least about 10⁻⁷ M, at least about 10⁻⁸ M, at least about 10⁻⁹ M, or lower. Those skilled in the art will appreciate that, in some cases, binding affinity (e.g., as measured by a dissociation constant) may be influenced by non-covalent intermolecular interactions such as hydrogen bonding, electrostatic interactions, hydrophobic and Van der Waals forces between the two molecules. Alternatively or additionally, binding affinity between a ligand and its target molecule may be affected by the presence of other molecules. Those skilled in the art will be familiar with a variety of technologies for measuring binding affinity and/or dissociation constants in accordance with the present disclosure, including, e.g., but not limited to ELISAs, gel-shift assays, pull-down assays, equilibrium dialysis, analytical ultracentrifugation, surface plasmon resonance (SPR), bio-layer interferometry, grating-coupled interferometry, and spectroscopic assays.

In some embodiments, competition assays may be used to identify an antibody that competes with the anti-MuSK antibody agents described herein for binding to the Ig3 domain of MuSK. In some embodiments, such a competing antibody binds to the same epitope within the Ig3 domain of MuSK that is bound by the anti-MuSK antibodies described herein. Exemplary epitope mapping methods are known. See, e.g., Morris (1996).

In some embodiments, assays can be provided for identifying anti-MuSK antibody agents thereof having biological activity. In some embodiments, assays can be provided for identifying anti-MuSK antibody agents thereof having neutralization activity for MuSK. Antibody agents having such biological activity in vivo and/or in vitro can be also provided. In some embodiments, an antibody of the disclosure can be tested for such biological activity.

The “biological activity” of an anti-MuSK antibody agent can refer to, for example, binding affinity for a particular MuSK epitope (e.g., within the Ig3 domain), neutralization or inhibition of MuSK binding to BMP, neutralization or inhibition of MuSK activity in vivo (e.g., IC₅₀), pharmacokinetics, and cross-reactivity (e.g., with non-human homologs or orthologs of the MUSK protein, or with other proteins or tissues). Other biological properties or characteristics of an antigen-binding agent recognized in the art can include, for example, avidity, selectivity, solubility, folding, immunotoxicity, expression, and formulation. The aforementioned properties or characteristics can be observed, measured, and/or assessed using standard techniques including, but not limited to, ELISA, competitive ELISA, surface plasmon resonance analysis (BIACORE™), or Kinetic Exclusion Assay (KINEXA™), in vitro or in vivo neutralization assays, receptor-ligand binding assays, cytokine or growth factor production and/or secretion assays, and signal transduction and immunohistochemistry assays.

In some embodiments, a MuSK MR agonizing agent as described herein is characterized in that, for example, the MuSK MR agonizing agent (e.g., an agonizing oligonucleotide), when contacted with a cell expressing MuSK, will increase the level or activity of MuSK ΔIg3 mRNA and/or protein.

In some embodiments, a MuSK MR agonizing oligonucleotide is characterized by its ability to alter splicing activity of MuSK pre-mRNA in a cell. For example, a cell may be transfected with a MuSK MR agonizing oligonucleotide, and after a period of incubation, expression of an alternative form of processed form of a MuSK RNA transcript (e.g., where exons 6 and 7 have been skipped), can be measured by RT-PCR. For example, the efficiency of MuSK exon skipping in cultured cells greater than 10%, 20%, 30%, 40%, 50%, 60%, 70%, 80%, 90%, 95%, or 95%.

In some aspects, a MuSK MR agonizing oligonucleotide increases of MuSK ΔIg3 mRNA. In some aspects, a MuSK MR agonizing oligonucleotide alters splicing of MuSK pre-mRNA. In some aspects, a MuSK MR agonizing oligonucleotide promotes the skipping of exon 6 and/or exon 7.

Modulation of expression of MuSK ΔIg3 can be measured in a bodily fluid of a subject treated with MuSK MR agonizing oligonucleotide, which may or may not contain cells; tissue; or organ of the animal. Methods of obtaining samples for analysis, such as body fluids (e.g., sputum, serum, CSF), tissues (e.g., biopsy), or organs, and methods of preparation of the samples to allow for analysis are well known to those skilled in the art. The effects of treatment on a subject can be assessed by measuring biomarkers associated with the target gene expression in one or more biological fluids, tissues or organs, collected from an animal contacted with one or more compositions described in this application.

In some embodiments, an increase in MuSK ΔIg3 mRNA means that the intracellular level of MuSK ΔIg3 mRNA is higher than a reference level, such as the level of MuSK ΔIg3 mRNA in a control (for example in a subject that is not being administered a MuSK MR agonizing oligonucleotide). An increase in intracellular MuSK ΔIg3 mRNA can be measured as an increase in the level of MuSK ΔIg3 protein and/or mRNA produced. In some embodiments, an increase in MuSK ΔIg3 mRNA can be determined by e.g., methods as described below in the examples, and/or by assay techniques such as RNA solution hybridization, nuclease protection, Northern hybridization, reverse transcription, gene expression monitoring with a microarray, antibody binding, enzyme linked immunosorbent assay (ELISA), nucleic acid sequencing, Western blotting, radioimmunoassay (RIA), other immunoassays, fluorescence activated cell analysis (FACS), or any other technique or combination of techniques that can detect the presence of MuSK ΔIg3 mRNA or protein (e.g., in a subject or a sample obtained from a subject).

In some embodiments, by comparing the level of MuSK ΔIg3 mRNA in a sample obtained from a subject receiving a MuSK MR agonizing oligonucleotide treatment to a level of MuSK ΔIg3 mRNA in a subject not treated with a MuSK MR agonizing oligonucleotide, the extent to which the MuSK MR agonizing oligonucleotide treatment increased MuSK ΔIg3 mRNA can be determined. In some embodiments, the reference level of MuSK ΔIg3 mRNA is obtained from the same subject prior to receiving MuSK MR agonizing oligonucleotide treatment. In some embodiments, the reference level of MuSK ΔIg3 mRNA is a range determined by a population of subjects not receiving MuSK MR agonizing oligonucleotide treatment. In some embodiments, the level of full-length MuSK mRNA is compared to the level of MuSK ΔIg3 mRNA. In some embodiments, the ratio of the MuSK ΔIg3 mRNA to a full length MuSK mRNA (e.g., MuSK mRNA without exons 6 and 7) in a subject receiving a MuSK MR agonizing oligonucleotide treatment, for example, greater than 1 fold, 1.5-5 fold, 5-10 fold, 10-50 fold, 50-100 fold, about 1.1-, 1.2-, 1.5-, 2-, 3-, 4-, 5-, 6-, 7-, 8-, 9-, 10-, 15-, 20-, 30-, 40-, 50-, 60-, 70-, 80-, 90-, 100-fold or more higher than a reference ratio.

In some embodiments, an increased level of MuSK ΔIg3 mRNA is, for example, greater than 1 fold, 1.5-5 fold, 5-10 fold, 10-50 fold, 50-100 fold, about 1.1-, 1.2-, 1.5-, 2-, 3-, 4-, 5-, 6-, 7-, 8-, 9-, 10-, 15-, 20-, 30-, 40-, 50-, 60-, 70-, 80-, 90-, 100-fold or more higher than a reference value.

In some embodiments, the increase of MuSK ΔIg3 mRNA in a subject can be indicated by the increase of MuSK ΔIg3 protein as compared to a reference level. In some embodiments, the reference level of MuSK ΔIg3 protein is the MuSK ΔIg3 protein level obtained from a subject having or at risk of having e.g., neuromuscular dysfunction, a neurodegenerative disorder, a cardiac disorder (e.g., myocardial infarction, cardiomyopathy), or genetic diseases characterized by muscle wasting, prior to treatment. Methods whereby bodily fluids, organs or tissues are contacted with an effective amount of one or more compositions described herein are also contemplated. Bodily fluids, organs or tissues can be contacted with one or more compositions comprising MuSK MR agonizing oligonucleotides, resulting in expression of MuSK ΔIg3 and modulation of MuSK expression in the cells of bodily fluids, organs or tissues. An effective amount of can be determined by monitoring the effect on functional MuSK ΔIg3 protein expression of MuSK MR agonizing oligonucleotides that are administered to a subject or contacted to a cell.

In some embodiments, a MuSK MR agonizing agent, when administered to a population of cells, (e.g., comprising satellite cells (SCs), myoblasts, myogenic progenitor cells (MPCs)), increases the number of cells that are in an activated state (e.g., active proliferation). Cells within a population can be assessed for whether they are in an activated state by known methods in the art, including e.g., an EdU assay, where EdU+ cycling cells are compared with total cell counts. In some embodiments, a MuSK MR agonizing agent, when administered to a population of cells comprising satellite cells, decreases the number of quiescent satellite cells in the population and/or increases the number of activated satellite cells.

In some embodiments, a MuSK MR agonizing agent, when administered to a population of cells comprising SCs, MPCs, and/or myoblasts, increases the number of cells expressing genes or myogenic factors (e.g., Pax7, MyoD, myogenin, and MERGE) and/or decreases the number of cells expressing genes associated with the MuSK-BMP signaling pathway (e.g., RGS4, Msx2, Myf5, Ptx3, Id1). In some embodiments, a MuSK MR agonizing agent, when administered to a population of cells comprising satellite cells and/or myoblasts, increases the level of expression of genes associated with myogenic factors (e.g., Pax7, MyoD, myogenin, and MERGE) and/or decreases the level of expression of genes associated the MuSK-BMP signaling pathway (e.g., RGS4, Msx2, Myf5, Ptx3, Id1) in the population of cells.

In some embodiments, a population of cells comprises satellite cells and/or myoblasts that have been induced to be satellite cells and/or myoblasts (e.g., from stem cells such as embryonic stems cells or pluripotent stem cells).

In some embodiments, a population of cells is obtained from a healthy subject. In some embodiments, a population of cells is obtained from a subject suffering from a disease or disorder such as a neuromuscular dysfunction, a neurodegenerative disorder, a cardiac disorder (e.g., myocardial infarction, cardiomyopathy), or genetic diseases characterized by muscle wasting.

In some embodiments, a MuSK MR agonizing agent, when contacted with a population of cells from a subject, increases muscle regeneration in a subject. In some embodiments, a MuSK MR agonizing agent is contacted with the population of cells in vivo, for example, by injection into a subject.

In some embodiments, a MuSK MR agonizing agent is contacted with the population of cells ex vivo by obtaining a population of cells from a subject, and muscle regeneration is increased when the treated cells are re-introduced into the subject.

In some embodiments, a MuSK MR agonizing agent, when administered to a subject, will increase muscle regeneration and/or growth and/or neuromuscular function, and/or myogenesis. Examples of methods to assess these biological effects are detailed, e.g., in the below examples.

### Model Systems

Among other things, the present disclosure provides model systems useful as described herein.

For example, in some embodiments, provided model system(s) that can be used to screen, validate, characterize, assess, and/or identify one or more MuSK MR agonizing agents.

In some embodiments, a model system provided herein is or comprises an artificially engineered cell line. In some embodiments, an engineered cell line is an immortalized MuSK^{-/-} myogenic cell line.

In some embodiments, a model system provided herein is or comprises an engineered mouse as described herein. In some embodiments, a provided mouse is or comprises an ΔIg3-MuSK mouse.

In some embodiments, provided model systems (e.g., cell lines and/or mice) are used to screen, validate, characterize, assess, and/or identify agents as described herein, including, for example, small molecule agents, antibody agents, oligonucleotide agents, etc., and combinations thereof. In some embodiments, activity of such agents is compared to an appropriate reference (e.g., a positive and/or a negative control). In some embodiments, an appropriate reference may be or comprise absence of any agent, or presence of an agent of known activity or performance in the model system. In some embodiments, an appropriate reference may be a historical reference. In some embodiments, an appropriate reference may be a contemporary or simultaneous reference.

### Production of Agonizing Agents

Antibodies

Antibodies and antigen-binding fragments of the present invention may be prepared and/or purified by any technique known in the art, which allows for the subsequent formation of a stable antibody or antibody fragment.

A nucleic acid encoding an anti-MuSK antibody agent of the present disclosure may be easily isolated and sequenced by conventional procedures.

In some embodiments, an expressed antibody of the present disclosure may be uniformly purified after being isolated from a host cell. Isolation and/or purification of an antibody of the present disclosure may be performed by a conventional method for isolating and purifying a protein. For example, not wishing to be bound by theory, an MuSK antibody agent of the present disclosure can be recovered and purified from recombinant cell cultures by well-known methods including, but not limited to, protein A purification, protein G purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan (1997-2001). In some embodiments, an antibody of the present disclosure may be isolated and/or purified by additionally combining filtration, superfiltration, salting out, dialysis, etc.

Purified anti-MuSK agents of the present disclosure can be characterized by, for example, ELISA, ELISPOT, flow cytometry, immunocytology, BIACORET™ analysis, SAPIDYNE KINEXAT™ kinetic exclusion assay, SDS-PAGE and Western blot, or by HPLC analysis as well as by a number of other functional assays disclosed herein.

Oligonucleotides

An agonizing agent, e.g., an agonizing oligonucleotide described herein can be synthesized by standard methods known in the art, e.g., by use of an automated synthesizer. Following chemical synthesis (e.g., solid-phase synthesis using phosphoramidite method), agonizing oligonucleotide molecules can be deprotected, annealed to ds molecules, and purified (e.g., by gel electrophoresis or HPLC). Protocols for preparation of agonizing oligonucleotides are known in the art.

An agonizing oligonucleotides can also be formed within a cell by transcription of RNA from an expression construct introduced into the cell. See, e.g., Yu et al., (2002). An expression construct for in vivo production of agonizing oligonucleotide molecules can include one or more antisense encoding sequences operably linked to elements necessary for the proper transcription of the antisense encoding sequence(s), including, e.g., promoter elements and transcription termination signals. Preferred promoters for use in such expression constructs include the polymerase-III HI-RNA promoter (see, e.g., Brummelkamp et al. (2002)) and the U6 polymerase-III promoter (see, e.g., Sui et al. (2002); Paul et al. (2002); and Yu et al. (2002). An agonizing oligonucleotide expression construct can further comprise one or more vector sequences that facilitate the cloning of the expression construct. Standard vectors that can be used include, e.g., pSilencer 2.0-U6 vector (Ambion Inc., Austin, Tex.).

### Pharmaceutical Compositions

The present disclosure provides pharmaceutical compositions that comprise and/or deliver agonizing agent(s) as described herein. The present disclosure also provides pharmaceutical compositions that are or comprise cell populations that have been exposed to agonizing agent(s) as described herein.

For example, in some embodiments, a provided pharmaceutical composition may comprise and/or deliver a MuSK MR agonizing agent such as, for example, an antibody agent or nucleic acid agent that, when administered, achieves an increase in level and/or activity of a MuSK polypeptide (e.g., a MuSK ΔIg3 polypeptide, or another MuSK variant polypeptide with disrupted Ig3) that lacks an Ig3 domain functional for interaction with BMP. Alternatively or additionally, in some embodiments, a provided pharmaceutical composition may comprise and/or deliver a population of cells that has been exposed to a MuSK MR agonizing agent, so that neuronal cell number and/or activity is increased in the population.

In many embodiments, a pharmaceutical composition will be or comprise an active agent (e.g., an agonizing agent as described herein or a precursor thereof) in combination with one or more pharmaceutically acceptable excipients. Those skilled in the art will appreciate that components of a particular pharmaceutical composition may be influenced by route of administration of the pharmaceutical composition.

The compositions of the disclosure can be formulated for a variety of modes of administration, including systemic and topical or localized administration. Techniques and formulations generally may be found in Remington (2000).

Compositions of the present invention can be prepared and administered in a wide variety of oral, parenteral, and topical dosage forms. Thus, the compositions of the present invention can be administered by injection (e.g., intravenously, intramuscularly, intracutaneously, subcutaneously, intraduodenally, or intraperitoneally). Also, the compositions described herein can be administered by inhalation, for example, intranasally. Additionally, the composition of the present invention can be administered transdermally. It is also envisioned that multiple routes of administration (e.g., intramuscular, oral, transdermal) can be used to administer the compositions of the invention.

In some embodiments, a pharmaceutical composition as described herein may be formulated for delivery by a route selected from intravenous injection, intrathecal administration, oral administration, buccal administration, inhalation, nasal administration, topical administration, ophthalmic administration or otic administration. In some embodiments, a pharmaceutical composition may be formulated for delivery by intrathecal administration. In some embodiments, a pharmaceutical composition may be formulated for delivery by intravenous administration. In some embodiments, a pharmaceutical composition may be formulated for delivery by oral administration.

In certain embodiments, oligonucleotides and compositions are delivered to the CNS. In certain embodiments, oligonucleotides and compositions are delivered to the cerebrospinal fluid. In certain embodiments, oligonucleotides and compositions are administered to the brain parenchyma. In certain embodiments, oligonucleotides and compositions are delivered to an animal/subject by intrathecal administration, or intracerebroventricular administration. Broad distribution of oligonucleotides and compositions, described herein, within the central nervous system may be achieved with intraparenchymal administration, intrathecal administration, or intracerebroventricular administration.

In certain embodiments, parenteral administration is by injection, by, e.g., a syringe, a pump, etc. In certain embodiments, the injection is a bolus injection. In certain embodiments, the injection is administered directly to a tissue, such as striatum, caudate, cortex, hippocampus and cerebellum.

In certain embodiments, methods of specifically localizing a pharmaceutical agent, such as by bolus injection, decreases median effective concentration (EC₅₀) by a factor of 20, 25, 30, 35, 40, 45 or 50. In certain embodiments, the pharmaceutical agent in an antisense compound as further described herein. In certain embodiments, the targeted tissue is brain tissue. In certain embodiments the targeted tissue is hippocampus tissue. In certain embodiments, decreasing EC₅₀ is desirable because it reduces the dose required to achieve a pharmacological result in a patient in need thereof.

In certain embodiments, an antisense oligonucleotide is delivered by injection or infusion once every month, every two months, every 90 days, every 3 months, every 6 months, twice a year or once a year.

In addition to the active ingredients, these pharmaceutical compositions may contain suitable pharmaceutically acceptable carriers comprising excipients and auxiliaries which facilitate processing of an active compound into preparations which can be used pharmaceutically. The preparations formulated for oral administration may be in the form of tablets, dragees, capsules, or solutions.

Pharmaceutical preparations for oral use can be obtained by combining an active compound with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, an active compound may be dissolved or suspended in suitable liquids, such as fatty oils, liquid paraffin, or liquid polyethylene glycols (PEGs). In addition, stabilizers may be added.

In some embodiments, the pharmaceutical composition is a tablet, a pill, a capsule, a liquid, an inhalant, a nasal spray solution, a suppository, a suspension, a gel, a colloid, a dispersion, a suspension, a solution, an emulsion, an ointment, a lotion, an eye drop or an ear drop.

Depending on the specific conditions being treated, pharmaceutical composition of the present disclosure may be formulated into liquid or solid dosage forms and administered systemically or locally. The pharmaceutical composition may be delivered, for example, in a timed- or sustained-low release form as is known to those skilled in the art. Techniques for formulation and administration may be found in Remington (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

For injection, the pharmaceutical composition of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compositions herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection.

In some embodiments, compositions as described herein can be formulated using pharmaceutically acceptable carriers available in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, one or more solubilizing, diluting, or dispersing substances such as, saline, preservatives, such as benzyl alcohol, absorption promoters, and fluorocarbons, may be employed.

In some embodiments, a provided composition may comprise and/or deliver a precursor of an active agent, wherein the precursor becomes or releases active therapeutic agent upon administration. In some embodiments, for example, a precursor may be or comprise a prodrug of a small molecule agonizing agent, or a nucleic acid that encodes a protein agonizing agent, etc.

In some particular embodiments, a provided pharmaceutical composition comprises or delivers a therapeutically effective amount (e.g., an amount that is effective when administered according to an established protocol) of a provided oligonucleotide (which may, as described herein, be provided in a pharmaceutically acceptable salt form, e.g., as a sodium salt, ammonium salt, etc.); in some embodiments, such as provided pharmaceutical composition includes a relevant oligonucleotide and at least one pharmaceutically acceptable inactive ingredient selected from pharmaceutically acceptable diluents, pharmaceutically acceptable excipients, and pharmaceutically acceptable carriers. In some embodiments, a salt form of a provided oligonucleotide comprises two or more cations, for example, in some embodiments, up to the number of negatively charged acidic groups (e.g., phosphate, phosphorothioate, etc.) in an oligonucleotide.

Pharmaceutically acceptable salts are generally well known to those of ordinary skill in the art, and may include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington, The Science and Practice of Pharmacy (20th ed. 2000). Preferred pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

As appreciated by a person having ordinary skill in the art, oligonucleotides may be formulated as a number of salts for, e.g., pharmaceutical uses. In some embodiments, a salt is a metal cation salt and/or ammonium salt. In some embodiments, a salt is a metal cation salt of an oligonucleotide. In some embodiments, a salt is an ammonium salt of an oligonucleotide. Representative alkali or alkaline earth metal salts include sodium, lithium, potassium, calcium, magnesium, and the like. In some embodiments, a salt is a sodium salt of an oligonucleotide. In some embodiments, pharmaceutically acceptable salts include, when appropriate, nontoxic ammonium, quaternary ammonium, and amine cations formed with counterions such as hydroxide, carboxylate, sulfate, phosphate, nitrate, sulfonate, phosphorothioate, etc. that may be within provided oligonucleotides. As appreciated by a person having ordinary skill in the art, a salt of an oligonucleotide may contain more than one cations, e.g., sodium ions, as there may be more than one anions within an oligonucleotide.

In some embodiments, provided oligonucleotides, and compositions thereof, may be effective over a wide dosage range. For example, in the treatment of adult humans, dosages from about 0.01 to about 1000 mg, from about 0.5 to about 100 mg, from about 1 to about 50 mg per day, and from about 5 to about 100 mg per day are examples of dosages that may be used. The exact dosage will depend upon the route of administration, the form in which the compound is administered, the subject to be treated, the body weight of the subject to be treated, and the preference and experience of the attending physician.

In some embodiments, the present disclosure provides technologies (e.g., compositions, methods, etc.) for combination therapy, for example, with other therapeutic agents and/or medical procedures. In some embodiments, provided oligonucleotides and/or compositions may be used together with one or more other therapeutic agents. In some embodiments, provided compositions comprise provided oligonucleotides, and one or more other therapeutic agents. In some embodiments, the one or more other therapeutic agents may have one or more different targets, and/or one or more different mechanisms toward targets, when compared to provided oligonucleotides in the composition. In some embodiments, a therapeutic agent is an oligonucleotide. In some embodiments, a therapeutic agent is a small molecule drug. In some embodiments, a therapeutic agent is a protein. In some embodiments, a therapeutic agent is an antibody. A number of a therapeutic agent may be utilized in accordance with the present disclosure. In some embodiments, provided oligonucleotides or compositions thereof are administered prior to, concurrently with, or subsequent to one or more other therapeutic agents and/or medical procedures. In some embodiments, provided oligonucleotides or compositions thereof are administered concurrently with one or more other therapeutic agents and/or medical procedures. In some embodiments, provided oligonucleotides or compositions thereof are administered prior to one or more other therapeutic agents and/or medical procedures. In some embodiments, provided oligonucleotides or compositions thereof are administered subsequent to one or more other therapeutic agents and/or medical procedures. In some embodiments, provide compositions comprise one or more other therapeutic agents.

### Production of Pharmaceutical Compositions

For preparing pharmaceutical compositions from the compositions of the present invention, pharmaceutically acceptable carriers can be either solid or liquid. Solid form preparations include powders, tablets, pills, capsules, cachets, suppositories, and dispersible granules. A solid carrier can be one or more substance that may also act as diluents, flavoring agents, binders, preservatives, tablet disintegrating agents, or an encapsulating material.

In powders, the carrier is a finely divided solid in a mixture with the finely divided active component. In tablets, the active component is mixed with the carrier having the necessary binding properties in suitable proportions and compacted in the shape and size desired.

The powders and tablets preferably contain from 5% to 70% of the therapeutic agent. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active therapeutic agent with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

When parenteral application is needed or desired, particularly suitable admixtures for compositions of the invention are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampoules are convenient unit dosages. The compositions of the invention can also be incorporated into liposomes or administered via transdermal pumps or patches. Pharmaceutical admixtures suitable for use in the present invention include those described, for example, in Pharmaceutical Sciences (17th Ed., Mack Pub. Co., Easton, PA) and WO 96/05309.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted according to the particular application and the potency of the active component. The composition can, if desired, also contain other compatible therapeutic agents.

Patient populations: In some embodiments, an appropriate patient or population is one suffering from and/or susceptible to a disease, disorder such as neuromuscular dysfunction, a cardiac disorder (e.g., myocardial infarction, cardiomyopathy), or genetic diseases characterized by muscle wasting or that otherwise would benefit from increased muscle regeneration).

In some embodiments, a subject and/or population may additionally or alternatively be suffering from and/or susceptible to a disease, disorder or condition that is a neurodegenerative disease, disorder, or condition. In some embodiments, such neurodegenerative disease, disorder, or condition is one or more of Alzheimer's Disease (AD), Parkinsons' disease, dementia (e.g., Frontotemporal dementia), stroke, Major Depressive Disorder (MDD), bipolar disorder, Schizophrenia, Post-Traumatic Stress Disorder (PTSD), substance-related and addictive disorders (e.g., chronic cocaine use and lifelong cigarette smoking), Temporal-Lobe Epilepsy, Hippocampal Sclerosis, Niemann Pick Type C, Diabetes-mediated hippocampal neuronal loss, and Huntington's disease.

In some embodiments, or population may additionally or alternatively be suffering from and/or susceptible to a disease or disorder of the lung. In some embodiments, such a disease or disorder is one or more of idiopathic pulmonary fibrosis (IPF), acute respiratory distress syndrome (ARDS), pneumonia, and lung complications due to viral infections.

In some embodiments, an appropriate patient or population is model organisms. In some embodiments, an appropriate patient or population is humans. In some embodiments, a human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In some embodiments, a human is a human infant. In some embodiments, a human is a human toddler. In some embodiments, a human is a human child. In some embodiments, a human is a human adult. In yet other embodiments, a human is an elderly human.

In some embodiments, an appropriate patient or population may be characterized by one or more criterion such as age group, gender, genetic background, preexisting clinical conditions, prior exposure to therapy.

In some embodiments, an appropriate patient or population is one suffering from e.g., neuromuscular dysfunction, a neurodegenerative disorder, a cardiac disorder (e.g., myocardial infarction, cardiomyopathy), or genetic diseases characterized by muscle wasting. In some embodiments, an appropriate patient or population is one suffering that has received surgery or experienced injury, trauma and/or prolonged immobilization (e.g., from bed-rest or casting). In some embodiments, an appropriate patient or population is one suffering from sarcopenia. In some embodiments, an appropriate patient or population is one suffering from or at risk of muscle fibrosis resulting from a disease or condition including, but not limited to, trauma, heritable disease, muscle disorder, and aging. Trauma can result from, for example, radiation treatment, crush injury, laceration, and amputation. In some embodiments, an appropriate patient or population is one suffering from or at risk of heritable disease associated with muscle fibrosis such as Congenital Muscular Dystrophy, Duchenne Muscular Dystrophy, Becker's Muscular Dystrophy, Amyotrophic Lateral Sclerosis (ALS), age-associated sarcopenia, Distal muscular dystrophy, Emery-Dreifuss muscular dystrophy, Facioscapulohumeral muscular dystrophy, Limb-girdle muscular dystrophy, Myotonic muscular dystrophy, and Oculo-pharyngeal muscular dystrophy.

In some embodiments, an appropriate patient or population maybe defined by those in accordance with the screening tools for diseases or disorders associated with muscle fibrosis and/or muscle wasting. In some embodiments, an appropriate patient or population maybe defined by those in accordance with the screening tools and methods for diagnosing a disease associated with muscle fibrosis and/or muscle wasting.

In some embodiments, an appropriate patient or population may be defined according to the results obtained in structural imaging (e.g., magnetic resonance imaging (MRI), computed tomography (CT), ultrasound etc.). In some embodiments, an appropriate patient or population may be defined according to the results of neurological tests. In some embodiments, the cognitive tests involve one or more tests of Motor Screening Task (MOT), Reaction Time (RTI), Paired Associates Learning (PAL), Spatial Working Memory (SWM), Pattern Recognition Memory (PRM), Delayed Matching to Sample (DMS), Rapid Visual Information Processing (RVP). Rapid Visual Information Processing (RVP), Delayed Matching to Sample (DMS), Match to Sample Visual Search (MTS). In some embodiments, an appropriate patient or population may be defined according to the results of assessments such as measuring muscle enzymes, EMG, muscle biopsy, genetic testing, heart testing (e.g., ECG), assessments of strength and respiratory function.

### Administration

Those skilled in the art will appreciate that, in some embodiments, dosage administered to a subject, particularly a human, may vary, for example depending on the particular therapeutic and/or formulation employed, the method of administration, the dosing regimen, one or more characteristics of the particular subject being treated, etc. In some embodiments, a clinician skilled in the art will determine the therapeutically effective amount of a therapeutic to be administered to a human or other subject in order to treat or prevent a particular medical condition. The precise amount of the therapeutic required to be therapeutically effective will depend upon numerous factors, e.g., such as the specific activity of the therapeutic, and the route of administration, in addition to many subject-specific considerations, which are within those of skill in the art.

In some embodiments, administration may be ocular, oral, buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc.

Those skilled in the art, reading the present disclosure will appreciate that, in some embodiments, it may be desirable to achieve delivery of a MuSK MR agonizing agent to muscle.

Alternatively or additionally, in some embodiments, it may be desirable to achieve delivery of a MuSK MR agonizing agent to the CNS (e.g., the brain, such as the hippocampus and/or the subventricular region) and/or to the lung.

In some embodiments, an agent (e.g., an agonizing agent) is delivered via systemic delivery and/or local delivery to muscle (e.g., via intramuscular injection).

In some embodiments, a MuSK MR agonizing agent is administered using a viral vector to effectively deliver a MuSK MR agonizing agent in the form of a nucleic acid payload. In some embodiments, a viral vector targets certain cell types (e.g., myoblasts, myocytes, myotubes, satellite cells and myofibers). AAV1, AAV6, and AAV9 vectors have been used to target different muscle cell types. See, for example, Arnett et al. (2014) and Riaz et al. (2015).

Those skilled in the art, reading the present disclosure will appreciate that, in some embodiments, it may be desirable to achieve delivery of a MuSK MR agonizing agent to the CNS, and, in some embodiments to the brain.

In some embodiments systemic administration achieves delivery to CNS (e.g., brain e.g., hippocampus and/or subventricular zone). In some embodiments, an agent (e.g., an agonizing agent) is delivered to the central nervous system (CNS), via intracerebroventricular administration.

Additionally, certain viral vectors are known to selectively target neurons, and to effectively deliver genetic payloads to the brain. For example, AAV2/1 vectors have been established to effectively deliver nucleic acid payloads (e.g., gene therapy, encoded RNAs, etc.) to neuronal cells in the hippocampus. See, e.g., Hammond et al. (2017); Guggenhuber et al. (2010); Lawlor et al. (2007). Analogously, certain AAV vectors (e.g., AAV2/1 and/or AAV4 vectors) have been established to target and effectively deliver nucleic acid payloads to certain cells in the subventricular zone cells. See, for example, Liu et al. (2005); Bockstael et al. (2012).

For subjects suffering from or susceptible to a disease, disorder or condition associated with neurodegeneration, administration that achieves delivery to the CNS, e.g., to the brain (e.g., to the hippocampus and/or the subventricular region) may be desirable.

In some embodiments, effective delivery may be achieved by systemic administration of a composition as described herein. Alternatively or additionally, in some embodiments, effective delivery may be achieved by local administration to the CNS and/or to the brain, for example by intrathecal and/or intracavitary (e.g., intracerebroventricular) delivery.

Technologies for local administration to the CNS and/or to the brain have been developed and demonstrated to be effective, for example, for various protein therapeutics (see, e.g., Calias et al., (2014)); for small molecules (see, e.g., Dodou (2012)); for cell compositions (see, e.g., Eftekharzadeh et al., (2015)); and nucleic acid therapeutics (see, e.g., Otsuka et al. (2011)); see also prescribing information for onasemnogene abeparvovec-xioi [sold under the brand name Zolgensma™] and that for nusinersen [sold under the brand name Spinraza™])

Those skilled in the art will be aware that intrathecal delivery may be particularly effective to achieve delivery to the hippocampus, including for cellular, protein, and nucleic acid therapeutics.

Systemic administration technologies (including, e.g., oral, parenteral, mucosal, etc.) are well established for a wide variety of agents. Systemic administration that achieves CNS and/or brain delivery, in some embodiments, may depend on ability to cross the blood brain barrier (BBB).

Certain active agents and/or delivery systems are known to cross the BBB. Recent technologies have been shown to achieve CNS and/or brain delivery even of agents, such as oligonucleotides, that had historically been considered to be particularly challenging in that regard. To give but one example, Min et al. (2020) describes glucose-coated polymeric nanocarriers that transport oligonucleotides across the BBB.

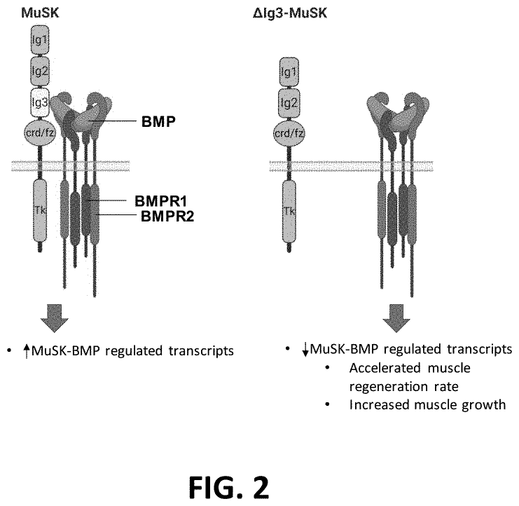

It has also been reported that incorporation of certain particular chemistries into oligonucleotide therapeutics can facilitate their travel across the BBB. For example, Khorkova et al., (2017) have described that:
“2′-modified phosphorothioate oligonucleotides . . . may be particularly adaptable for CNS disorders, given their long half-life, with effects in the brain lasting up to 6 months following a single injection. In another type of sugar moiety modification, locked nucleic acids (LNAs), a bridge is introduced that connects the 2′ oxygen and 4′ carbon. This modification substantially elevates the melting temperature of the LNA-DNA and LNA-RNA hybrids, thus allowing the creation of shorter ODN-based compounds with increased bioavailability and reduced manufacturing costs. A recently proposed tricyclo-DNA, a conformationally constrained oligonucleotide analog, has three additional C-atoms between C(5′) and C(3′) of the sugar (FIG. 2). This modification increases stability, hydrophobicity and RNA affinity, and improves tissue uptake and BBB permeability”.
   (citations omitted).

For subjects suffering from or susceptible to a disease or disorder such as idiopathic pulmonary fibrosis (IPF), acute respiratory distress syndrome (ARDS), pneumonia, and lung complications due to viral infections, administration that achieves delivery to the lungs may be desirable.

Oligonucleotides

In some embodiments, ASOs are developed to enhance their delivery to target site(s). As described in the art, the oligo load is covalently bound to a carrier or ligand, such as lipid particles, liposomes, nanoparticles, and more recently, the sugar N-acetyl galactosamine to enhance safer delivery to the target site. See, Verma (2018).

Certain technologies have been developed to improve the efficiency of cellular delivery of ASOs to target site, e.g., muscle. For example, aminoglycosides (AGs) are shown to improve the delivery of antisense phosphorodiamidate morpholino oligomer (PMO) both in vitro and in vivo. See, Wang, et al. (2019). Short cell-penetrating peptides (CPPs) that can be either directly attached to oligonucleotides through covalent linkages or through the formation of noncovalent nanoparticle complexes can facilitate cellular uptake. See, McClorey and Banerjee (2018). ASO fatty acid conjugates are also reported to enhance the functional uptake of antisense oligonucleotide (ASO) in the muscle. See, Prakash et al. (2019).

Those skilled in the art will be familiar with eteplirsen (ExonDys 51), an approved treatment for Duchenne muscular dystrophy (DMD), which is a third-generation phosphorodiamidate morpholino ASO.

Eteplirsen, sold under the brand name Exondys51™, (Sarepta Therapeutics’) causes exon 51 to be spliced out in pre-mRNA, restoring the reading frame in the 13% of patients with amenable frame-shifting mutations. See, Crudele and Chamberlain (2019).

Eteplirsen is administered via intravenous infusion over 35 to 60 minutes. In particular, its recommended dosage is 30 mg/kg body weight weekly. In a single-dose vial, the pharmaceutical composition is formulated as a 100 mg/2 mL or 500 mg/mL (50 mg/mL) solution.

In some embodiments, an oligonucleotide therapeutic as described herein may be administered intravenously. In some such embodiments, such oligonucleotide therapeutic may be administered according to a regimen reasonably comparable to that used for eteplirsen [sold under the brand name Exondys51™].

In some embodiments a lower dose of an agonizing oligonucleotide as described herein is 12 mg. In some embodiments, a total of 5 mg to 60 mg per dose of agonizing oligonucleotide is administered to a subject. In some embodiments, a total of 12 mg to 48 mg per dose of agonizing oligonucleotide is administered to a subject. In some aspects, a total of 12 mg to 36 mg per dose of agonizing oligonucleotide is administered to a subject. In some aspects, a total of 12 mg per dose of agonizing oligonucleotide is administered to a subject.

Those skilled in the art will be familiar with nusinersen [sold under the brand name Spinraza™] an antisense oligonucleotide therapeutic that targets the survival motor neuron-2 (SMN2)-directed gene transcript and is indicated for the treatment of spinal muscular atrophy (SMA) in pediatric and adult patients. Spinraza is administered intrathecally. In particular, its recommended dosage is 12 mg/5 mL (2.4 mg/mL) in a single-dose vial per administration, according to a regiment that involves four loading doses; the first three of which are administered at 14-day intervals, and the fourth of which is administered 30 days after the 3rd dose; a maintenance dose is administered once every 4 months thereafter. It is recommended that platelet count, coagulation laboratory testing, and quantitative spot urine protein testing is done at baseline, and prior to each dose.

In some embodiments, an oligonucleotide therapeutic as described herein may be administered intrathecally. In some such embodiments, such oligonucleotide therapeutic may be administered according to a regimen reasonably comparable to that used for nusinersen [sold under the brand name Spinraza™].

In some embodiments, an oligonucleotide therapeutic as described herein may be administered intrathecally. In some such embodiments, such oligonucleotide therapeutic may be administered according to a regimen reasonably comparable to that used for nusinersen [sold under the brand name Spinraza™].

Cell Therapy

In light of the ability of MuSK MR agonizing agents, as described herein, to promote muscle regeneration (e.g., from cell populations that are or comprise SCs, MPCs, and/or myoblasts), those skilled in the art reading the present disclosure will appreciate that, among other things, the present disclosure provides technologies for enhancing level of SCs, MPCs, and/or myoblasts present in a cell population. That is, contacting an original cell population with a MuSK MR agonizing agent as described herein can generate a resulting population with an increased level and/or percentage of SCs, MPCs, and/or myoblasts as compared with that in the original population; administration of such MuSK MR agonizing agent as described herein can achieve such increase.

In some embodiments, an original cell population may be or comprise SCs, MPCs, and/or myoblasts. In some embodiments, an original cell population is or comprises embryonic stems cells and/or pluripotent stem cells. In some embodiments, embryonic stems cells and/or pluripotent stem cells are or have been differentiated into myogenic progenitor cells, for example using techniques known in the art. See, e.g., Miyagoe-Suzuki et al. (2017).

In some embodiments, as discussed above, such administration delivers the MuSK MR agonizing agent such that it is exposed to (i.e., contacts) a relevant original cell population in vivo (e.g., in a human, and in particular in an adult human, for example into muscle tissue, of such human).

In some embodiments, administration in accordance with the present disclosure contacts a MuSK MR agonizing agent with a population of cells (e.g., an original population of cells), that for example, may be or comprise SCs, MPCs, and/or myoblasts, ex vivo. For example, in some embodiments, a MuSK MR agonizing agent is administered ex vivo (e.g., in vitro) to a population of cells from a subject. In some embodiments, a population of cells obtained from a subject.

In some embodiments, a MuSK MR agonizing agent of particular use ex vivo may be or comprise a small molecule, and antibody, or a nucleic acid agent, or a combination thereof. In some particular such embodiments, one or more agents that is or comprises a nucleic acid (e.g., one of more gene therapy [e.g., nucleic acid vector and/or transcript), oligonucleotide, and/or gRNAs) may be particular useful for ex vivo and/or in vitro administration to cells. CRISPR/Cas modification of cell populations is an established and growing field, and those skilled in the art will appreciate applicability of such strategies in accordance with the present disclosure, e.g., to modify and/or disrupt MuSK Ig3 domain sequences. Alternatively or additionally, nucleic acids that encode (or whose expression products encode) MuSK forms lacking a functional Ig3 domain may be introduced into cells ex vivo and/or in vitro. Still further alternatively or additionally, oligonucleotides that direct exon skipping of MuSK transcript(s) to favor forms that lack functional Ig3, and/or that direct degradation (and/or block translation) of forms that include functional Ig3, may be utilized.

In some embodiments, a population of cells is contacted with a MuSK MR agonizing agent and simultaneously or subsequently stimulated and/or expanded. Alternatively or additionally, a population of cells is enriched and/or selected for cells exhibiting characteristics of activated satellite cells or for expression of myogenic factors (e.g., Pax7, MyoD, myogenin, and MERGE) or for decreased/lack of expression of genes associated with the MuSK-BMP signaling pathway (e.g., RGS4, Msx2, Myf5, Ptx3, Id1).

In some embodiments, a resulting population of cells, achieved by contacting an original population of cells with a MuSK MR agonizing agent ex vivo is then administered to a subject. In some embodiments, a resulting population of cells is administered to a subject suffering from or susceptible to a disease or disorder such as a neuromuscular dysfunction, a neurodegenerative disorder, a cardiac disorder (e.g., myocardial infarction, cardiomyopathy), or genetic diseases characterized by muscle wasting. In some embodiments, a resulting population of cells is administered to the subject from whom the original population of cells was obtained. In some embodiments, a resulting population of cells is administered to a different subject than the one from which the original population of cells was obtained; in some such embodiments, the original population was obtained from a healthy subject and the resulting population is administered to a subject suffering from or susceptible to a disease or disorder such as a neuromuscular dysfunction, a neurodegenerative disorder, a cardiac disorder (e.g., myocardial infarction, cardiomyopathy), or genetic diseases characterized by muscle wasting.

In some embodiments, administering a population of cells, contacted with a MuSK MR agonizing agent effectively treats a disease or disorder such as a neuromuscular dysfunction, a neurodegenerative disorder, a cardiac disorder (e.g., myocardial infarction, cardiomyopathy), or genetic diseases characterized by muscle wasting in the subject.

In some embodiments, a population of stimulated and/or expanded SCs, MPCs, and/or myoblasts described herein can be formulated into a cellular therapeutic. In some embodiments, a cellular therapeutic includes a pharmaceutically acceptable carrier, diluent, and/or excipient. Pharmaceutically acceptable carriers described herein, for example, vehicles, adjuvants, excipients, and diluents, are well-known and readily available to those skilled in the art. Preferably, the pharmaceutically acceptable carrier is chemically inert to the active agent(s), e.g., a cellular therapeutic, and does not elicit any detrimental side effects or toxicity under the conditions of use.

In some embodiments, a cellular therapeutic can be formulated for administration by any suitable route, such as, for example, intravenous, intratumoral, intraarterial, intramuscular, intraperitoneal, intrathecal, epidural, and/or subcutaneous administration routes. Preferably, the cellular therapeutic is formulated for a parenteral route of administration. In some embodiments, a cellular therapeutic is administered to a subject via an infusion.

In some embodiments, a cellular therapeutic suitable for parenteral administration can be an aqueous or non-aqueous, isotonic sterile injection solution, which can contain anti-oxidants, buffers, bacteriostats, and solutes, for example, that render the composition isotonic with the blood of the intended recipient. An aqueous or nonaqueous sterile suspension can contain one or more suspending agents, solubilizers, thickening agents, stabilizers, and preservatives.

In some embodiments, a single therapeutic cell described herein is capable of expanding and providing a therapeutic benefit. In some embodiments, 10² or more, e.g., 10³ or more, 10⁴ or more, 10⁵ or more, or 10⁸ or more, therapeutic cells are administered as a cellular therapeutic. Alternatively, or additionally 10¹² or less, e.g., 10¹¹ or less, 10⁹ or less, 10⁷ or less, or 10⁵ or less, therapeutic cells described herein are administered to a subject as a cellular therapeutic. In some embodiments, 10²-10⁵, 10⁴-10⁷, 10³-10⁹, or 10⁵-10¹¹ therapeutic cells described herein are administered as a cellular therapeutic.

A dose of a cellular therapeutic described herein can be administered to a subject at one time or in a series of sub doses administered over a suitable period of time, e.g., on a daily, semi-weekly, weekly, bi-weekly, semi-monthly, bi-monthly, semi-annual, or annual basis, as needed. A dosage unit comprising an effective amount of a cellular therapeutic may be administered in a single daily dose, or the total daily dosage may be administered in two, three, four, or more divided doses administered daily, as needed. In some embodiments, a cellular therapeutic is administered in combination with another therapy.

### Combination Therapy

In some embodiments, MuSK MR agonizing therapy as described herein is administered in combination with another therapy, i.e., so that a subject is simultaneously exposed to both therapies.

The dosage of the MuSK MR agonizing therapy as described herein and the dosage of another therapy administered in combination, as well as the dosing schedule can depend on various parameters, including, but not limited to, the disease being treated (e.g., a neuromuscular dysfunction, a neurodegenerative disorder, a cardiac disorder, or a genetic disease characterized by muscle wasting), the subject's general health, and the administering physician's discretion.

MuSK MR agonizing therapy can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the other therapy, to a subject in need thereof. In various embodiments MuSK MR agonizing therapy and the other therapy are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, MuSK MR agonizing therapy and the other therapy are administered within 3 hours. In another embodiment, MuSK MR agonizing therapy and the other therapy are administered at 1 minute to 24 hours apart.

A synergistic combination of MuSK MR agonizing therapy and the other therapy, might allow the use of lower dosages of one or both of these agents and/or less frequent administration of the therapies to a subject suffering from neuromuscular dysfunction, a neurodegenerative disorder, a cardiac disorder, or genetic diseases characterized by muscle wasting. A synergistic effect might result in the improved efficacy of these agents and/or the reduction of any adverse or unwanted side effects associated with the use of either agent alone.

In some embodiments, MuSK MR agonizing therapy is administered in combination with a standard of care treatment for a relevant disease, disorder, or condition (e.g., a neuromuscular dysfunction, a neurodegenerative disorder, a cardiac disorder, or genetic diseases characterized by muscle wasting).

Therapies for DMD include deflazacort (Emflaza; PTC Therapeutics) eteplirsen (Exondys 51; Sarepta Therapeutics), Ataluren (Translarna; PTC Therapeutics), and glucocorticoids such as prednisone. In some embodiments, MuSK MR agonizing therapy is administered in combination with one or more therapies for DMD.

Approved therapies for ALS include Radicava, Rilutek, Tiglutik, and Nuedexta. In some embodiments, MuSK MR agonizing therapy is administered in combination with one or more therapies for ALS.

Approved therapies for cardiomyopathy include but are not limited to angiotensin II-converting enzyme (ACE) inhibitors, angiotensin II receptor blockers (ARBs) and spironolactone. In some embodiments, MuSK MR agonizing therapy is administered in combination with one or more therapies for cardiomyopathy.

In some embodiments, MuSK MR agonizing therapy is administered in combination with one or more therapies that relieves a symptom or characteristic of a relevant disease, disorder or condition, or of a therapy therefor. In some embodiments, MuSK MR agonizing therapy is administered in combination with one or more other therapies that relieves a symptom or characteristic so that the side effects associated with said other therapies are relieved. In some embodiments, the side effect associated with therapy is characterized by one or more of muscle cramps and spasms, constipation, fatigue, excessive saliva and phlegm, pain, depression, sleep problems, and uncontrolled outbursts of laughing or crying.

Any therapy which is known to be useful, or which has been used, will be used or is currently being used for the treatment or prevention of neuromuscular dysfunction, a neurodegenerative disorder, a cardiac disorder, or genetic diseases characterized by muscle wasting, can be used in combination with the MuSK MR agonizing therapy in accordance with the invention described herein.

### EXEMPLIFICATION

### Example 1: Role of the MuSK-BMP Pathway in Regulating Satellite Cell Dynamics and Muscle Regeneration In Vivo

### Regeneration in Constitutive ΔIg3-MuSK Muscle

The BMP signaling pathway promotes muscle regeneration and satellite cell proliferation in vivo. Several lines of evidence support a role for the MuSK-BMP pathway in this process. MuSK levels are upregulated in whole muscle during regeneration. We previously reported that the MuSK-BMP pathway regulates the expression of a number of transcripts encoding myogenic factors in cultured myoblasts and myotubes. Satellite cells have been reported to express MuSK mRNA, but the presence of MuSK protein in these cells has not been reported.

To determine if satellite cells express MuSK protein, immunohistochemistry (IHC) was performed on intact myofibers isolated from mouse hind-limb muscles and stained for: Pax7, a satellite cell-specific protein in the muscle; MyoD, expressed in activated satellite cells; and MuSK. As shown in FIG. 1C, activated, but not quiescent, satellite cells express detectable MuSK protein, which is similar to the expression pattern of BMP in satellite cells.

### ΔIg3-MuSK Mice

In order to study MuSK-BMP signaling in vivo, CRISPR/Cas9 was used to generate mice that have a constitutive deletion of the Ig3 domain of MuSK (ΔIg3-MuSK mice), the domain necessary for high-affinity BMP binding.

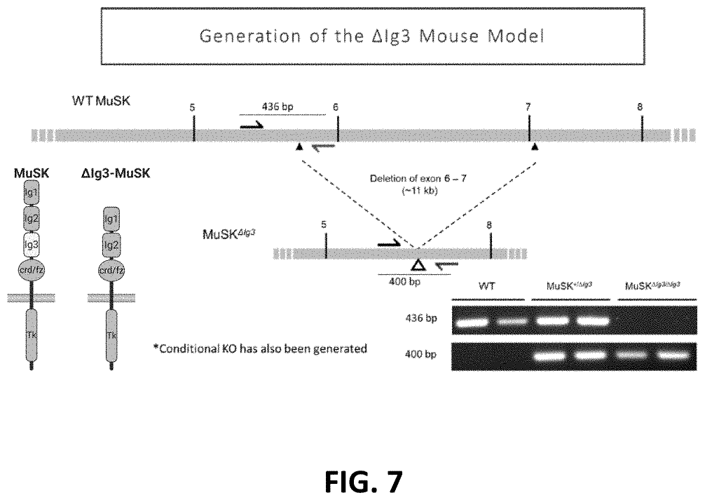

A mouse model that lacks the MuSK Ig3 domain was generated using CRISPR/Cas9 (FIG. 7). Plasmids (▴) encoding hSpCas9 and gRNAs flanking the locus that encodes the Ig3 domain were designed to excise an approximate 11 kb region thereby generating a novel MuSK allele (MuSK^{ΔIg3}) that lacks the Ig3 encoding domain. Arrows indicate PCR primers designed to amplify either the WT or MuSK^{ΔIg3}. gDNA sequences used to target sequences within the MuSK gene are shown below:

(SEQ ID NO: 3)
Musk_sgRNAex6up1: TGCTCATATCTAAATGCGAT
(SEQ ID NO: 4)
Musk_sgRNAex7dw1: GCACTCCATGGCATCTGGAA
(SEQ ID NO: 5)
Musk_sgRNAex6up2: GAGCATAAATGTTCTAGACT
(SEQ ID NO: 6)
Musk_sgRNAex7dw2: CTCCATGGCATCTGGAAGGG

Mice that were homozygous for MuSK^{ΔIg3} were selected by genotyping and confirmed by DNA sequencing. Amplification of genomic DNA of the WT and MuSK^{ΔIg3} alleles by PCR produces amplicons of 436 and 400 bp, respectively. WT mice have the WT MuSK allele but not the MuSK^{ΔIg3}. Heterozygous MuSK^{ΔIg3} mice have both WT and MuSK^{ΔIg3} alleles as evidenced by 436 and 400 bp products, respectively, while MuSK^{ΔIg3} homozygotes only amplify the 436 bp MuSK^{ΔIg3} allele (FIG. 7).

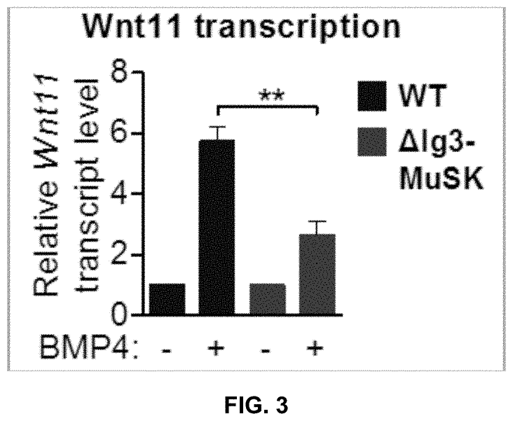

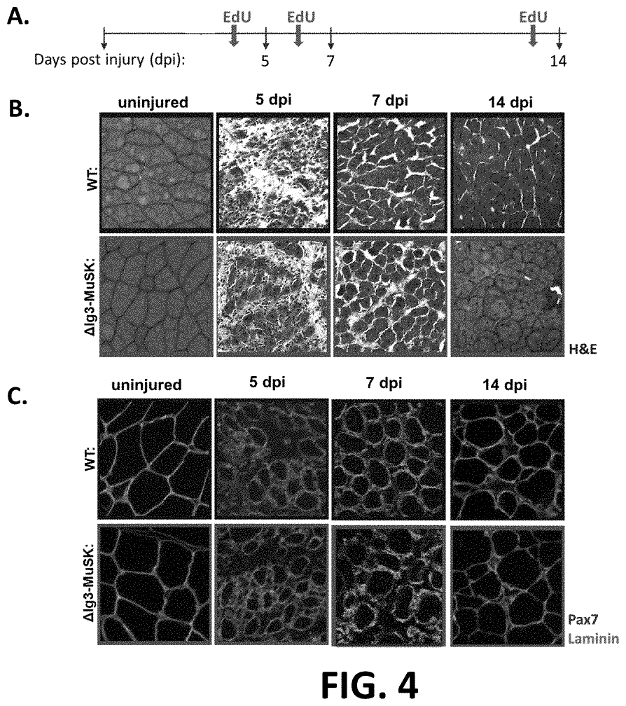

Our studies showed that that MuSK-BMP signaling is perturbed in primary myotubes cultured from ΔIg3-MuSK mice. Significantly reduced levels of MuSK-BMP regulated transcripts were measured; for example, transcription of Wnt11 was defective (see FIG. 3). In vivo studies also indicated that MuSK-BMP signaling was disrupted in these mice (see FIG. 4B-C and FIG. A-B).

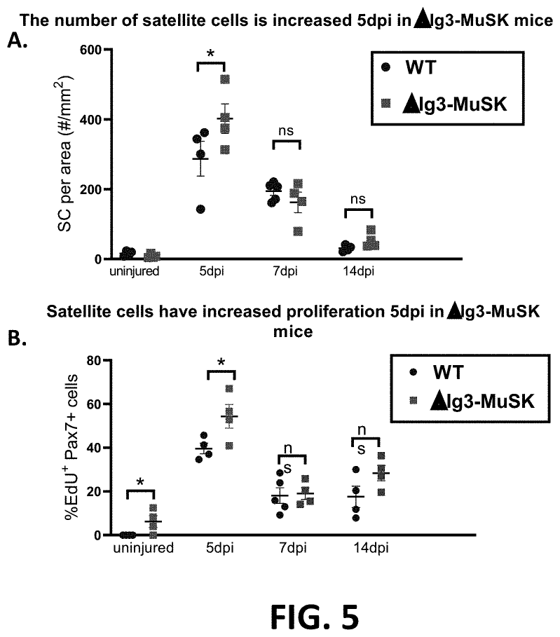

ΔIg3-MuSK mice were also used to examine satellite cells in uninjured and injured, regenerating muscle. As shown in FIG. 4A, satellite cell numbers were found to be equivalent in uninjured tibialis anterior (TA) and soleus muscles between ΔIg3-MuSK and WT mice, suggesting that the MuSK-BMP pathway is not required for satellite cell development. In contrast, as shown in FIG. 4B-C and FIG. A-B, satellite cell amounts 5-dpi were higher in ΔIg3-MuSK compared to WT muscle. To test if this difference was due to increased proliferation, mice were injected with 5-ethynyl-2′-deoxyuridine (EdU) on day 4 and satellite cell proliferation was observed to be significantly higher in ΔIg3-MuSK muscle compared to WT muscle between 4 and 5 dpi (FIG. 5B). These results suggest that MuSK-BMP signaling regulates satellite cell proliferation after injury.

The same regeneration experiment described above was performed but tissues were harvested from mice at 7 dpi and 14 dpi (a time at which muscle regeneration is near completion) and results are also shown in FIG. 4B-C and FIG. 5A. As shown in FIG. 4D and FIG. 5B, satellite cell numbers returned near baseline at 7 and 14 dpi.

Together, these data indicate that MuSK-BMP signaling regulates satellite cell activity during muscle regeneration and suggest that constitutive ΔIg3-MuSK mice will have altered muscle regeneration due to abnormal satellite cell proliferation and differentiation.

### Methods

Tibialis anterior muscle from constitutive ΔIg3-MuSK and WT mice were injured using a 1.2% BaCl₂ (in PBS) injections. Injected BaCl₂ destroys muscle fibers but leaves the basal lamina and satellite cells intact, which allows for regeneration. For uninjured controls, both TA contralateral to the injured muscle as well as TA muscle of a separate, uninjected littermate mouse were used to control for effects of systemic factors released by injured muscle. Mice that were 5 months old (a time when full muscle growth has been achieved) were used. Regeneration was analyzed at four time points: 5 dpi, when satellite cell activation is at its peak; 7 dpi and 14 dpi, when muscle is nearly regenerated; and 21 dpi, which is enough time to allow for complete muscle regeneration in WT mice. Mice are perfusion-fixed because the Pax7 antibody gives optimal staining under these conditions (see FIG. 4C).

To assess muscle regeneration, TA muscle were harvested and weighed. IHC is performed by staining with DAPI, laminin to show the cell membrane of the myofibers, and embryonic Myosin Heavy Chain (emb-MyHC). The number of centrally nucleated myofibers and the number of myofibers that express emb-MyHC were counted, both of which are markers of regenerated myofibers. The weight of the injured muscle was compared to the control muscles and the percent of control muscle weight was calculated. The sizes of the regenerated myofibers was also calculated using laminin staining and morphometry with Image software.

Satellite cell dynamics was also compared at each time point and the total number of satellite cells and the percentage of satellite cells were determined in each stage (quiescent, proliferating, and differentiating). EdU (5-ethynyl-2′-deoxyuridine) was injected intraperitoneally for 1-3 days prior to mouse harvest and then the EdU+ cells after harvest were detected using the Click-it EdU cell proliferation assay kit (Thermo Fisher Scientific) in order to mark the satellite cells that were actively proliferating during the days that EdU is administered. Muscle sections were also stained for quiescent and differentiating satellite cells for their respective, unique protein expression profiles, as outlined in FIG. 1A. All antibodies are commercially available or sourced from the Developmental Studies Hybridoma Bank. Differences in total satellite cell number or percentage of satellite cells that are quiescent, proliferating, or differentiating between ΔIg3-MuSK and WT muscle suggest that MuSK-BMP signaling controls satellite cell dynamics during muscle regeneration. To confirm that BMP signaling is active, a read out of BMP signaling is obtained by staining for phosphorylated-Smad1/5/8. Potential CRISPR/Cas9 off-target effects are controlled for by repeating key studies in a second, independent ΔIg3-MuSK line that has been generated.

### Regeneration in Conditional Satellite Cell-ΔIg3-MuSK Muscle

In addition to using constitutive ΔIg3-MuSK mice, mice that, upon tamoxifen injection, will conditionally excise the Ig3 domain of MuSK in satellite cells (SC-ΔIg3-MuSK mice) are also generated.

MuSK is expressed in intact muscle and upregulated during regeneration. The tamoxifen inducible, conditional SC-ΔIg3-MuSK mice is used to determine whether results are due to MuSK-BMP satellite cell autonomy, as opposed to contribution from MuSK-BMP signaling from other cell types. This conditional mouse model also informs if MuSK-BMP signaling regulates satellite cell functional development since disrupted MuSK-BMP activity can be induced after the mouse has reached adulthood. Conditional SC-ΔIg3-MuSK mice exhibit altered muscle regeneration due to abnormal SC proliferation and differentiation after injury.

### Methods

Mice that have tamoxifen-inducible excision of the Ig3 domain of MuSK in satellite cells are generated and referred to as conditional SC-ΔIg3-MuSK mice. MuSK-Ig3^{loxP/loxP} mice have been generated in which the Ig3 domain of MuSK is flanked with LoxP sites. The Pax7CreERT2; nuclear-tdTomato mouse is provided Dr. Bradley Olwin. In the conditional SC-ΔIg3-MuSK mice generated from crossing these two strains, Pax7 is expressed normally, and CreERT2 is expressed under the Pax7 promotor. Upon tamoxifen administration to induce CreERT2 activity, the Ig3 domain of MuSK is excised in all Pax7+ cells, thus excised in all satellite cells. Additionally, these mice express nuclear tdTomato under the Pax7 promoter in the safe harbor locus, Rosa26. This TdTomato labelling eases analysis because Pax7 antibody staining will be unnecessary and fibers derived from satellite cells will express tdTomato.

Using SC-ΔIg3-MuSK mice, muscle injury and regeneration assays and analysis are carried out as described above, except that mice are injected with an appropriate dose of tamoxifen every day for 5 days prior to injury to induce Cre-recombination.

### Statistical Analyses

T-tests are performed with appropriate post hoc corrections for multiple comparisons (e.g., Bonferroni). The number of animals required for the experiments was determined by performing a power analysis using G*Power (version 3.1) using preliminary data. These calculations indicate sufficient powering for observing significance (FIG. 5A 5 dpi, n=4 mice/group, effect size (d)=2.13). Using G*Power, it was determined that 7 animals per group was optimal to observe and effect size (d) of 2.13 (a=0.05; power=0.95).

### Results

Both the constitutive ΔIg3-MuSK muscles and conditional SC-ΔIg3-MuSK muscles show altered regeneration ability compared to WT control muscles after injury and dysregulated satellite cell proliferation and differentiation is observed due to disruption of MuSK-BMP signaling. Results described above show that these mice have increased satellite cell proliferation during regeneration, which indicates that normal MuSK-BMP signaling prevents muscle hypertrophy. Further, these findings show that constitutive ΔIg3-MuSK muscles and conditional SC-ΔIg3-MuSK muscles have aberrant regenerated weight, numbers of regenerated fibers, and/or sizes of regenerated myofibers when collected 14 and 21 days after injury in comparison to the WT muscle. Constitutive and conditional ΔIg3-MuSK mice also have aberrant amounts of quiescent, proliferating, and differentiating satellite cells at all time points. A lack of significant differences in results between the constitutive and conditional ΔIg3-MuSK mice would indicate that MuSK-BMP signaling during satellite cell development does not contribute to the injury phenotype, suggesting that any defect is satellite cell-dependent and autonomous.

Alternatively, there is no difference in final muscle regeneration, but the timing and/or satellite cell dynamics in the constitutive ΔIg3-MuSK TA muscles and conditional SC-ΔIg3-MuSK TA are different when compared to WT. This indicates that MuSK-BMP signaling regulates satellite cell dynamics, but other pathways compensate to ensure muscle is regenerated correctly, indicating that MuSK is a regulator of satellite cell dynamics.

### Fiber Size Assessment in Regenerating Wild Type and ΔIg3-MuSK Muscle

### After Injury

As described above, the phenotypes observed in regenerating ΔIg3-MuSK muscle are increased satellite cell numbers and proliferation at 5 dpi. Increased muscle fiber diameter is an indicator of accelerated regeneration. Accordingly, the present study compared muscle fiber size in regenerating WT and ΔIg3-MuSK TA muscles.

WT and ΔIg3-MuSK TA muscles were collected 7 days after BaCl₂ injury of 5-month old mice. The muscles were frozen in Optimal Cutting Temperature (OCT) compound and stored at −80° C. Muscles were cryosectioned into 10 microns sections onto charged slides that were then stained for Dapi (4′,6-diamidino-2-phenylindole; VECTASHIELD® Antifade Mounting Medium with DAPI) and Laminin, using rabbit polyclonal anti-Laminin from abcam (ab11575). Slides were imaged at 20× using a fluorescent microscope. Images were analyzed with the computer program MyoVision to measure fiber sizes and minimum Feret's diameter. Fiber outlining by the program was done manually. The DAPI staining was used to ensure that only fibers that were regenerating (i.e., centrally nucleated) were analyzed, as opposed to fibers that were uninjured. 1000-2000 fibers were analyzed per genotype from four different animals.

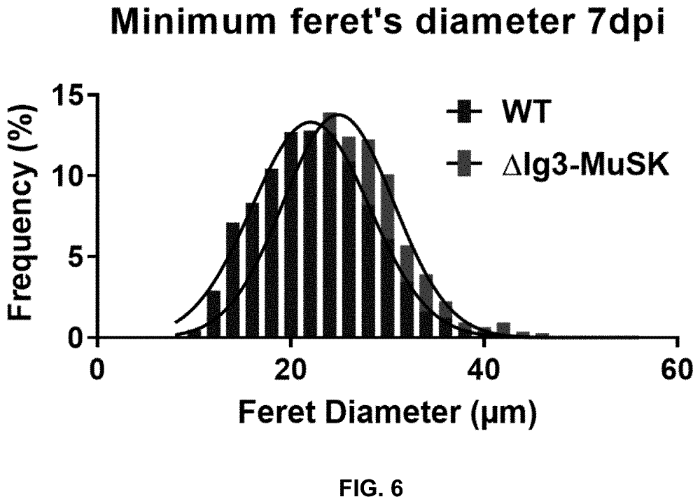

As shown in FIG. 6, the minimum Feret's diameter was significantly increased in regenerating ΔIg3-MuSK 7 dpi compared to WT muscle. This increased diameter is an indication of accelerated regeneration.

### Uninjured Mice

In addition, myofiber size and satellite cell number were assessed in uninjured WT and ΔIg3-MuSK mice.

WT and ΔIg3-MuSK TA muscles were collected from WT and ΔIg3-MuSK on 3 and 5 month old animals. The muscles were frozen in Optimal Cutting Temperature (OCT) compound and stored at −80° C. Muscles were cryosectioned into 10 microns sections onto charged slides that were then stained for Dapi (4′,6-diamidino-2-phenylindole; VECTASHIELD® Antifade Mounting Medium with DAPI) and Laminin, using rabbit polyclonal anti-Laminin from abcam (ab11575). Slides were imaged at 20× using a fluorescent microscope. Images were analyzed with the computer program MyoVision to measure fiber sizes and minimum Feret's diameter. Fiber outlining by the program was done manually. The DAPI staining was used to ensure that only fibers that were regenerating (i.e., centrally nucleated) were analyzed, as opposed to fibers that were uninjured. 1000-2000 fibers were analyzed per genotype from four different animals.

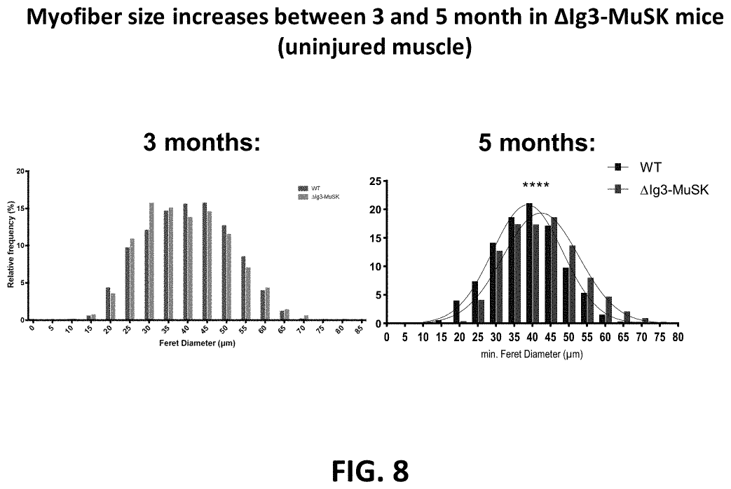

As shown in FIG. 8, at 3 months, no difference was observed in average myofiber size or size distribution between WT and ΔIg3-MuSK mice. Significant difference was observed at 5 months. However, specifically, 5-month-old ΔIg3-MuSK mice have increased myofiber size relative to 5-month-old WT mice. This finding indicates that, as they aged from 3 months to 5 months, the ΔIg3-MuSK mice had increased muscle growth relative to the WT mice. Mann-Whitney T-test: P<0.0001, n=3-4 mice, 500 total fibers analyzed per mouse.

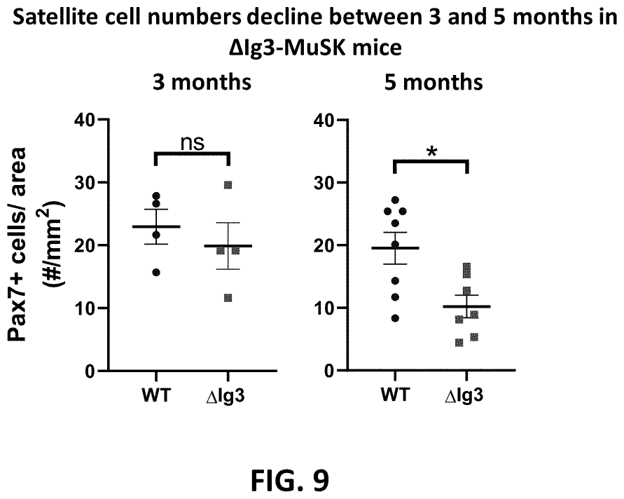

Additionally, immunohistochemistry was performed on the muscle sections determine the number of satellite cells, as measured by Pax7+ cells per area. Results shown in FIG. 9 show that in 3 month old animals, the numbers of satellite cells were equivalent between WT and ΔIg3-MuSK muscles. At 5 months, the amount of SCs in WT muscle was unchanged, while the amount of satellite cells in Ig3-MuSK muscle was reduced by about half. Each point represents an animal. Unpaired t-test, n=8 mice, p=0.01.

As described herein, muscle regeneration is characterized by explosive proliferation of satellite cells, for example as is observed in the 5 dpi mice described above. Muscle growth, by contrast, is characterized by decreased satellite cell numbers, as growth occurs when satellite cells differentiate and fuse with each other and/or with existing muscle fibers (resulting in increased fiber size, as is observed for the uninjured ΔIg3-MuSK of FIG. 8).

Thus, findings described herein demonstrate both that MuSK modulators (e.g., MuSK agonizing agents), can be useful both in contexts of muscle injury, repair and/or regeneration, and also in contexts of muscle growth (e.g., in muscle atrophy and/or muscle development).

### Example 2: Role of the MuSK-BMP Pathway in Regulating the Satellite Cell Transcriptome

Microarray analysis was performed on immortalized WT and MuSK−/− myogenic cell lines showed that MuSK modulates the magnitude and composition of the transcriptional output of these cells. A number of MuSK-regulated regulated genes discovered in this study are also known to regulate myogenesis, such as inhibitor of differentiation 1 (Id1) and Myf5. As shown in FIG. 3, we have now determined that the transcription of key MuSK-BMP dependent genes is also reduced in primary myotubes derived from neo-natal constitutive ΔIg3-MuSK mice. As noted above, BMP signaling promotes muscle regeneration, promotes satellite cell proliferation, and is downregulated in differentiating satellite cells. The data shown in FIG. 4C-D and FIG. 5A indicates that disrupted MuSK-BMP activity causes aberrant satellite cell proliferation during muscle regeneration in vivo.

RNA sequencing (RNA seq) analysis of satellite cells from conditional SC-ΔIg3-MuSK mice can be used to probe MuSK-BMP mechanism. In preparation for these studies, an existing RNA seq dataset from NCBI's Gene Expression Omnibus (GEO) database (GSE121589) was analyzed, which included RNA seq of satellite cells from uninjured and from injured, regenerating muscle. The raw data files were reprocessed by trimming adapters and low quality reads using TrimGalore!, and then the data were aligned to the ENSEMBL GRCm38 mouse genome using HiSat2. StringTie was used to determine the transcripts per million (TPM) of genes in each sample and DESeq2 was used to identify differently expressed genes (DEGs).

Key MuSK-BMP-dependent genes were found highly regulated in WT satellite cells in regenerating muscle, indicated by significant change in TMP from satellite cells of uninjured muscle (see Table 1). Combined with the data described herein (FIGS. 4-5), these data suggest that conditional SC-ΔIg3-MuSK mice provides valuable insights into the genes regulated by the MuSK-BMP pathway in satellite cells.

TABLE 1

| | Uninjured | 5 dpi | Log2 fold | |
|---|---|---|---|---|
| Gene | TPM | TPM | change | p-adjusted |
| MuSK | 32.64 | 56.42 | −0.34 | 0.621 |
| RGS4 | 155.99 | 34.12 | 2.68 | 8.04E−09 |
| Msx2 | 0.5323 | 0.0299 | 4.14 | 0.00165 |
| Myf5 | 38.22 | 203.57 | −2.090 | 0.000562 |
| Ptx3 | 32.39 | 5.49 | 3.088 | 5.54E−05 |
| Id1 | 242.19 | 95.20 | 1.89 | 6.15E−08 |

Using conditional SC-ΔIg3-MuSK mice, the transcriptomes of primary quiescent satellite cells (Example 2A) and activated proliferating satellite cells (Example 2B) isolated from uninjured and injured muscle, are examined. This study unveils MuSK-BMP regulated transcriptome in satellite cells. Some of the same MuSK-BMP regulated genes identified in immortalized myoblasts and primary myotubes are identified in this study. For example, Id1 has higher expression in WT compared to Ig3-MuSK−/− satellite cell as this gene is a downstream target of BMPs; it is three-fold more activated in BMP treated WT compared to MuSK−/− myoblasts; and it is highly expressed in proliferating satellite cells. Id1 is known to negatively regulate MyoD and myogenin and is thus downregulated in order for satellite cells to differentiate. The DEGs of WT and ΔIg3-MuSK satellite cells are compared at baseline and after injury. The MuSK-BMP pathway is more active in proliferating satellite cells than in quiescent satellite cells and MuSK-BMP transcripts, such as Id1, regulate satellite cell activity during regeneration.

### Genes Regulated by MuSK BMP Signaling in Satellite Cells

To identify genes regulated by MuSK-BMP signaling in satellite cells, RNA-Seq is first performed on quiescent satellite cells from uninjured muscles. Uninjured mouse hind limb muscles are pooled to ensure a sufficient satellite cell yield, since satellite cells make up a small percentage of total muscle nuclei in uninjured muscle. Satellite cells are isolated from tamoxifen-induced and non-tamoxifen-induced conditional SC-ΔIg3-MuSK mice. Use of tamoxifen-induced and non-induced conditional SC-ΔIg3-MuSK mice generates satellite cells with WT MuSK or ΔIg3-MuSK expression, as well as tdTomato expression under the Pax7 promotor. For simplicity, these are referred as WT and ΔIg3-MuSK SCs. tdTomato expression in these satellite cells are used for cell sorting with flow cytometry using a method optimized for these cells. The Pax7CerERT2 allele was bred in mice with a floxed nuclear tdTomato and these have successfully used this for live cell tracking. This yields about 100-150K cells per mouse. These mice are bred with the floxed ΔIg3-MuSK mice to study the specific effects of reduced MuSK-BMP signaling in satellite cells.

In consultation with the Computational Biology Core (CBC), RNA seq and subsequent bioinformatics are carried out to identify MuSK-BMP-dependent genes that are differentially expressed in satellite cells when the MuSK-BMP pathway is disrupted. cDNA libraries are prepared using TruSeq RNA V2 (Illumina) library preparation kit. RNA integrity is checked prior to library construction. Since the genes of interest represent a small portion of the transcriptomes that are sequenced, approximately 50 million reads per sample are sequenced. Data analysis are conducted as previously done on the publicly-available GEO dataset, described above, and with assistance from the CBC. A list of DEGs between WT and ΔIg3-MuSK SCs is compiled and the role of the Ig3 domain of MuSK on gene expression in quiescent satellite cells is evaluated. The ten most relevant DEGs are validated by qRT-PCR. An Ingenuity Pathway Analysis (Qiagen) is also performed using this data as a means to explore the role of the MuSK-BMP pathway in satellite cells.

### Further Probe of Genes Regulated in Satellite Cells of Regenerating Muscle

To further probe the MuSK-BMP pathway to determine which genes are regulated in satellite cells of regenerating muscle, RNA seq analysis is conducted on WT and ΔIg3-MuSK satellite cells collected from injured, regenerating TA muscle. The same muscle injury protocol described in Example 1 is used, except the TA is injured from both hind limbs to increase satellite cell number. Satellite cells are examined at 5 dpi because satellite cell proliferation is reported to be at its peak at this time and, as shown in FIG. 4E, at 5 dpi, satellite cells have aberrant satellite cell proliferation in injured Ig3-MuSK muscle. At 5 dpi, satellite cell number increased over 30 fold in comparison to satellite cell number in uninjured muscle (see FIG. 5A) and, which shows that regenerating TA muscle at 5 dpi has enough satellite cells for this purpose. Satellite cells are isolated using flow cytometry and then RNA seq and subsequent analysis are performed as described in Example 2A.

A potential pitfall to this approach is that the quiescent satellite cells are sorted from pooled hind limb muscles to ensure a high enough yield, but the activated satellite cells are sorted only from regenerating TA muscle. To address any concerns of muscle-fiber type effect, the only predominantly slow twitch muscle in the mouse hind limb, the soleus, can be excluded. Regenerating TA muscle yields a sufficient number of activated satellite cells, but the gastrocnemius muscle can be selected in order instead to increase cell yield further.

Additionally, satellite cells begin to activate immediately after harvest. Thus, it is possible that the cells described as “quiescent” from this study may actually be in the earliest stages of activation. To address this, high resolution in situ hybridization (ISH) is performed on select MuSK-BMP regulated transcripts to confirm that they are present in quiescent satellite cells. As an alternative approach to this study, primary satellite cell cultures are generated from these mice and RNA seq is performed with and without BMP4 treatment to probe the MuSK-BMP pathway. However, quiescent satellite cells cannot be examined with this method and, since satellite cells in culture are not in their niche, MuSK regulation of activation functions differently than it would in vivo.

### Statistical Powering:

The CBC offers services to help perform power analysis to determine the appropriate n. Prior to the above-described RNA seq experiments, the CBC is consulted to determine an appropriate n to use for RNA seq experiments.

### Results

The above-described results suggest that the Ig3 domain of MuSK regulates the expression of BMP-regulated genes (FIG. 2). This includes genes known to regulate satellite cell dynamics and myogenesis, such as Id1 and Myf5, as well as other novel genes that have not been previously identified as implicated in muscle regeneration. Furthermore, differences in the transcriptome between WT and ΔIg3-MuSK satellite cells are greater in satellite cells from regenerating muscle (i.e., activated proliferating satellite cells) in comparison to uninjured muscle (i.e., quiescent satellite cells). This is because the MuSK-BMP pathway regulates satellite cell proliferation and differentiation, and this pathway is not implicated in quiescence since no detectable MuSK was observed in quiescent satellite cells on muscle fibers (FIG. 1C).

### Example 3: Role of the MuSK-BMP Pathway in Muscle Fibrosis

Muscle fibrosis is the disruption of functional parenchyma by stromal elements. It's an often overlooked sequela of traumatic muscle injury, ageing, and congenital disease.

The remarkable regenerative capacity of skeletal muscle is dependent on the interaction of myogenic progenitors and the same stromal connective tissue elements responsible for fibrosis generation and propagation. Despite a remarkable capacity for regeneration, fibrotic replacement of functional muscle by stromal elements has been well documented in response to trauma, heritable disease, and aging. Indeed, muscle fibrosis poses a significant clinical problem for patients following radiation treatment, crush injury, laceration, and amputation, resulting in progressive loss of function and significant morbidity.

Extracellular matrix (ECM) is an essential component of skeletal muscle. It provides a framework structure that holds myofibers and blood capillaries and nerves supplying the muscle. In addition, it has a principal role in force transmission, maintenance and repair of muscle fibers. Excessive accumulation of ECM components, especially collagens, either due to excessive ECM production, alteration in ECM-degrading activities, or a combination of both is defined as fibrosis.

Recent studies have started elucidating the biomolecular mechanisms underlying the balance of muscle regeneration and muscle fibrosis. While the deposition of extracellular elements, and proliferation of stromal cells following injury are thought to underlie the pathogenesis of fibrosis formation, those same elements have proven indispensable for successful regeneration after injury, suggesting a critical and well-orchestrated balance between functional muscle tissue and the connective tissue that surrounds it.

Skeletal muscle fibrosis impairs muscle function, negatively affects muscle regeneration after injury and increases muscle susceptibility to re-injury. As such, it is considered a major cause of muscle weakness. Fibrosis of skeletal muscle is a hallmark of muscular dystrophies, aging and severe muscle injuries. Fibrosis also has a major role in many muscle disorders including Congenital, Duchenne and Becker's Muscular Dystrophy; age-associate sarcopenia; repair after muscle injury; Amyotrophic Lateral Sclerosis (ALS, also known as Lou Gehrig's Disease), and other muscle wasting conditions. Thus, a better understanding of the mechanisms of muscle fibrosis will help to advance knowledge of the events that occur in dystrophic muscle diseases and develop innovative anti-fibrotic therapies to reverse fibrosis in such pathologic conditions. Furthermore, agents that mitigate fibrosis would be beneficial in these conditions.

The MuSK-BMP pathway regulates satellite cell proliferation and satellite cell differentiation, suggesting it plays an important role in muscle regeneration. The present example explored whether this pathway is also implicated in muscle fibrosis.

Tibialis anterior muscles were injured with BaCl2 injection. At 5, 7, and 14-days post-injury (dpi), samples of wild type and ΔIg3-MuSK muscle were obtained and stained either with hematoxylin and eosin (H&E) or antibodies to the extracellular matrix protein laminin. As shown in FIG. 4B, at 14 dpi, the WT myofibers in the H&E stained muscle are separated by ‘clear’ spaces whereas the myofibers in the ΔIg3-MuSK muscle are tightly packed with minimal extracellular space. Such spacing in H&E is characteristic of accumulated extracellular matrix. The laminin staining confirms this interpretation. As can be seen in FIG. 4C, the ‘space’ between the myofibers contains abundant extracellular matrix, as shown by the interstitial immunoreactivity.

These data suggests that the MuSK-BMP pathway also plays an important role in muscle fibrosis and the third immunoglobulin domain of MuSK, Ig3, provides a therapeutic target for preventing and/or treating muscle fibrosis.

### Example 4: MuSK MR Agonizing Oligonucleotides

Design and synthesize of exon-skipping ASOs. Without wishing to be bound by any theory, MuSK MR agonizing oligonucleotides as described herein are designed in accordance with, but not limited to, the following general guidelines (see, Aartsma-Rus et al., 2012):
RNA or DNA modified for resistance to endo- or exonucleases (e.g., 2′MoE, 2′OMe, PMO, phosphorthioate);
Designed against target sequence;
Typically between 15-25 nucleotides, more optimally between 17-20;
Typically most effective with melting temperature of over 48° C.;
Typically most effective with GC content between 40% and 60% to prevent steric hindrance/dimerization, availability to access target;
Typically most effective targeting open/accessible pre-mRNA structures;
Typically most effective targeting splice regulatory sites or exon definition sites (e.g., intronic splice enhancers, intronic splice silencers (e.g., Spinraza, targeted against ISS of SMN2 exon 7, exonic splice enhancers, exonic splice silencers);
Typically most effective with sequence composition containing no more than 2 guanine (G) or cytosine (C) nucleotides in direct succession (e.g., CCC or GGG).

ASO chemistry. We will develop 2′-O-2-methoxyethyl (2′MOE) ASOs that also include phosphorothioate bonds in the sugar backbone. Methods for the design and testing of such ASOs are well-established, including manufacturing, pharmacokinetics, biodistribution and toxicology in rodents and non-human primates (Bennett and Swayze, 2010; Chiriboga et al., 2016; Hua et al., 2015; Mercuri et al., 2018; Rigo et al., 2014). The MOE group added to the 2′ position of the ribose increases Tm by about 2° C. per residue, thus elevating the binding affinity, and also improving nuclease resistance. The phosphorothioate modification confers further nuclease resistance and also increases affinity for plasma proteins, resulting in ASOs that efficiently distribute to tissues and are taken up into cells with need for formulations. This chemistry is off-patent, offering commercial advantages.

ASO Design. ASOs will be synthesized by a commercial facility and will be provided by Bolden Therapeutics, Inc. to the Fallon and Webb labs at Brown University for screening. The strategy for designing the ASOs will include scanning exonic and intronic sequences flanking both the 5′ and 3′ splice sites with overlapping ASOs (1-2 bp shift/oligo). The optimal length of the ASOs is ^{˜}17mer which provides a good balance between target specificity and drug exposure. ASOs will be pre-screened in silico for potential off-target effects as well as compositional bias (GC content) and propensity for unwanted dimer formation.

The ASOs will be designed such that they induce skipping of both exons 6 and 7 in MuSK (FIG. 3). Importantly, these two exons are coordinately spliced in vivo (Garcia-Osta et al., 2006; Hesser et al., 1999).

Screening and selection of optimal exon-skipping ASOs

We Will Design RT-qPCR TaqMan Assays to Specifically Quantify the Following Distinct MuSK splice forms: 1) full length (FL) MuSK; 2) Δexon, which is the desired product encoding ΔIg3-MuSK; and 3) potential ‘incomplete’ skipping’ isoforms Δexon and Δexon. We will perform conventional RT-PCR in parallel to detect any unexpected products. All screens will be carried out in murine C2C12 myoblasts. This cell line expresses MuSK endogenously and is efficiently transfected using standard methods such as Lipofectamine 2000. Cells will be transfected with candidate ASOs at several concentrations across a range from ˜0.1 to 10 nM. After One Day of Treatment RNA Will be Extracted and Splicing Will be measured by RT-qPCR to assess exon-skipping efficiency. Our goal is to isolate at least one ASO that induces ≥80% coordinate splicing of exons 6 and 7.
Testing the Selected ASOs for their Ability to Inhibit MuSK-BMP Signaling in Cultured Cells.

We have observed that knock-in mice constitutively expressing only ΔIg3-MuSK show increases in satellite cells after injury compared to WT mice, and have increased muscle fiber size, indicating enhance muscle regeneration. However, since skipping in vivo will likely be less than 100% efficiency (Rigo et al., 2014), it is important to establish the relationship between the level of skipping achieved and the physiological impact. Therefore, in this aim we will measure the level of MuSK-BMP dependent signaling in ASO-treated cells.

We will use qRT-PCR to measure the levels of MuSK-BMP dependent transcripts (e.g., Dok7 and Wnt11; FIG. 4, or RGS4; Yilmaz et al., 2016). We have extensive experience in this system gained during the discovery and characterization of MuSK as a BMP co-receptor (Yilmaz et al., 2016). Cells treated with either exon-skipping of control ASOs will be stimulated with BMP for 2 hr. The levels of transcripts will then be measured and the response to BMP will be correlated with the degree of exon skipping.

Data from the ΔIg3-MuSK mouse provided herein support that increased expression of ΔIg3-MuSK provides beneficial impact. The present disclosure appreciates that, in some embodiments, efficiency of skipping exons 6 and 7 may be insufficiently efficient with a single ASO. In some embodiments, it may be desirable to prepare one or more ASOs directed against exon 7, for use alone and/or with an exon 6-directed ASO.

Those of ordinary skill in the art, reading the present disclosure, will appreciate that, in some embodiments, it may be desirable to replicate studies (e.g., at least three times), and/or to analyze data with appropriate statistical methodologies (e.g., by t-tests with appropriate correction for multiple comparisons (e.g., Bonferroni)).

Work described herein provides technologies for efficient development of an ASO-mediated therapy for neuromuscular diseases and disorders and enhance muscle regeneration.

### Example 5: Immortalized ΔIg3-MuSK Cell Line

This example provides a ΔIg3-MuSK cell line to be used as a model system to screen, validate, characterize, assess, and/or identify one or more MuSK MR agonizing agents described herein in vitro.

To generate the ΔIg3-MuSK cell line, primary myoblasts were isolated from hindlimbs of neonatal wild type and MuSK-Ig3^{-/-} mice with at least one copy of the immortalizing transgene H-2Kb-tsA58 (Pimentel et al., 2017, Morgan et al., 1994). Cells were cultured in DMEM growth medium containing 20% FBS, 1% penicillin/streptomycin, 2% L-glutamine, 1% chick embryo extract, and 1% IFN-γ, at 33° C. and 10% CO₂. Cells were subcloned by plating at a density of 0.5 cells/well into a 96-well plate coated with Matrigel. Individual clones were chosen for expansion based on morphology, expanded, and tested to confirm ability to grow on gelatin substrate. Myogenicity was tested by plating in differentiation medium (DMEM with 5% horse serum, 1% penicillin/streptomycin) at 37° C. and 10% CO₂ and myogenic clones were expanded.

This cell line can be used to screen, validate, characterize, assess, and/or identify agents as described herein, including, for example, small molecule agents, antibody agents, oligonucleotide agents, etc., and combinations thereof. For example, the immortalized ΔIg3-MuSK myogenic cell line can be used in high through-put screening of small molecule MuSK MR agonizing agents. Example small molecule MuSK MR agonizing agents include small molecules that target one or more of type I BMP receptors, ALK3 (ALK is Anaplastic lymphoma kinase) and ALK6, and type I activin receptor ALK4, for example, an ALK inhibitor (e.g., crizotinib, ceritinib, alectinib, brigatinib, lorlatinib).

Gene expression profiles of the ΔIg3-MuSK cell line can be observed in response to treatment/exposure with particular MuSK MR agonizing agents, e.g., to determine the effects and characterize said agents on muscle growth and regeneration. Cellular assays to measure proliferation and differentiation markers may be used to characterize the MuSK MR agonizing agents.

### REFERENCES

Aartsma-Rus, A., et al., (2012). Overview on AON design. Methods Mol. Biol. 867: 117-129.
Akins, M. R., et al., (2017). Axonal ribosomes and mRNAs associate with fragile X granules in adult rodent and human brains. Human molecular genetics 26: 192-209.
Allen, T. M., (2002). Ligand-targeted therapeutics in anticancer therapy. Nature Reviews Cancer 2:750-763.
Altschul et al., (1990). Basic local alignment search tool. J. Mol. Biol., 215(3): 403-410.
Altschul et al., (1997). Methods in Enzymology; Altschul et al., Nucleic Acids Res. 25:3389-3402.
Arnett, A. L. H., et al., (2014). Adeno-associated viral vectors do not efficiently target muscle satellite cells. Molecular Therapy—Methods & Clinical Development (2014) 1, 14038.
Baxevanis et al., (1998). BIOINFORMATICS: A PRACTICAL GUIDE TO THE ANALYSIS OF GENES AND PROTEINS, Wiley.
Bennett, C. F. and Swayze, E. E. (2010). RNA targeting therapeutics: molecular mechanisms of antisense oligonucleotides as a therapeutic platform. Annu. Rev. Pharmacol. Toxicol. 50: 259-293.
Bentzinger, C. F., et al., (2012). Building muscle: molecular regulation of myogenesis. Cold Spring Harbor PERSPECTIVES IN BIOLOGY 4.
Bockstael, O., et al., (2012). Rapid Transgene Expression in Multiple Precursor Cell Types of Adult Rat Subventricular Zone Mediated by Adeno-Associated Type 1 Vectors. Human Gene Therapy 23: doi.org/10.1089/hum.2011.216,
Berge, S. M., et al., (1977). Pharmaceutical salts. J. Pharmaceutical Sciences, 66: 1-19.
Bowen, D. C., et al., (1998). Localization and regulation of MuSK at the neuromuscular junction. Developmental Biology 199: 309-319.
Brummelkamp, T. R., et al., (2002). A system for stable expression of short interfering RNAs in mammalian cells. Science; 296:550-553.
Calias, P., et al., (2014). Intrathecal delivery of protein therapeutics to the brain: A critical reassessment. Pharmacology & Therapeutics, 114(2), 114-122.
Charville, G. W., et al., (2015). Ex Vivo Expansion and In Vivo Self-Renewal of Human Muscle Stem Cells. Stem Cell Reports 5: 621-632.
Chiriboga, C. A., et al., (2016). Results from a phase 1 study of nusinersen (ISIS-SMN(Rx)) in children with spinal muscular atrophy. Neurology 86(10): 890-897.
Chothia C. and Lesk, A. M. (1987). Canonical structures for the hypervariable regions of immunoglobulins. J. Mol. Biol. 196(4): 901-917.
Chothia C., et al., (1989). Conformations of immunoglobulin hypervariable regions. Nature 342(6252): 877-883.
Cohen, S., et al., (2015). Muscle wasting in disease: molecular mechanisms and promising therapies. Nature reviews. Drug discovery 14: 58-74.
Collins, C. A., et al., (2005). Stem cell function, self-renewal, and behavioral heterogeneity of cells from the adult muscle satellite cell niche. Cell 122: 289-301.
Colligan, (1997-2001). Current Protocols in Immunology, or Current Protocols in Protein Science, John Wiley & Sons, NY, N.Y., e.g., chapters 1, 4, 6, 8, 9, and 10.
Crudele, J. M. and Chamberlain, J. S. (2019). AAV-based gene therapies for the muscular dystrophies. Human Molecular Genetics, 28: R102-R107.
CURRENT PROTOCOLS IN IMMUNOLOGY (CPI) (2003). John E. Coligan, A D A M Kruisbeek, David H Margulies, Ethan M Shevach, Warren Strobe, (eds.) John Wiley and Sons, Inc. (ISBN 0471142735, 9780471142737).
CURRENT PROTOCOLS IN MOLECULAR BIOLOGY (CPMB), (2014). Frederick M. Ausubel (ed.), John Wiley and Sons (ISBN 047150338X, 9780471503385).
CURRENT PROTOCOLS IN PROTEIN SCIENCE (CPPS), (2005). John E. Coligan (ed.), John Wiley and Sons, Inc.
Davis et al., (2012). BASIC METHODS IN MOLECULAR BIOLOGY, Elsevier Science Publishing, Inc., New York, USA (ISBN 044460149X).
DeChiara, T. M., et al., (1996). The receptor tyrosine kinase MuSK is required for neuromuscular junction formation in vivo. Cell 85: 501-512.
Dodou, K., (2012). Intrathecal route of drug delivery can save lives or improve quality of life. Pharm J. 289: 501.
Eftekharzadeh, M., et al., (2015). The effect of intrathecal delivery of bone marrow stromal cells on hippocampal neurons in rat model of Alzheimer's disease. Iran J. Basic Med. Sci. 18: 520-525.
Egner, I. M., et al., (2016). Satellite cell depletion prevents fiber hypertrophy in skeletal muscle. Development 143: 2898-2906.
Faul, F., et al., (2009). Statistical power analyses using G*Power 3.1: tests for correlation and regression analyses. Behavior Research Methods 41: 1149-1160.
Garcia-Osta, A., et al., (2006). MuSK Expressed in the Brain Mediates Cholinergic Responses, Synaptic Plasticity, and Memory Formation. Journal of Neuroscience 26 (30): 7919-7932.
Gozo, M. C., et al., (2013). Foxc2 induces Wnt4 and Bmp4 expression during muscle regeneration and osteogenesis. Cell death and differentiation 20: 1031-1042.
Green, M. R. and Sambrook, J. (2012). MOLECULAR CLONING: A LABORATORY MANUAL, 4th ed., Cold Spring Harbor Laboratory Press, Cold Spring Harbor, N.Y., USA (ISBN 1936113414).
Groffen, A. J., et al., (1998). Primary structure and high expression of human agrin in basement membranes of adult lung and kidney. Eur. J. Biochem. 254(1): 123-128.
Guggenhuber, S., et al., (2010). AAV Vector-Mediated Overexpression of CB1 Cannabinoid Receptor in Pyramidal Neurons of the Hippocampus Protects against Seizure-Induced Excitoxicity. PLoS One 5:e15707.
Hammond, S. L., et al., (2017). Cellular selectivity of AAV serotypes for gene delivery in neurons and astrocytes by neonatal intracerebroventricular injection. PLoS One 12:e0188830.
Harris, J. B. (2003). Myotoxic phospholipases A2 and the regeneration of skeletal muscles. Toxicon: Official Journal of the International Society on Toxinology 42: 933-945.
Hesser, B. A., et al., (1999). Identification and characterization of a novel splice variant of MuSK. FEBS Letters 442: 133-137.
https://vectorlabs.com/vectashield-mounting-medium-with-dapi.html
https://www.abcam.com/laminin-antibody-ab11575.html
https://www.myovision.com
Hua, Y. et al., (2015). Motor neuron cell-nonautonomous rescue of spinal muscular atrophy phenotypes in mild and severe transgenic mouse models. Genes Dev. 29(3): 288-297.
Ibebunjo, C. and Martyn, J. (2001). Disparate dysfunction of skeletal muscles located near and distant from burn site in the rat. Muscle & nerve 24: 1283-1294.
IMMUNOLOGY, (2006). Werner Luttmann, published by Elsevier.
Isselbacher, et al., (1996). HARRISON'S PRINCIPLES OF INTERNAL MEDICINE, 13 ed., 1814-1882.
JANEWAY'S IMMUNOBIOLOGY, (2014). Kenneth Murphy, Allan Mowat, Casey Weaver (eds.), Taylor & Francis Limited, (ISBN 0815345305, 9780815345305).
Janssen, I., et al., (2000). Skeletal muscle mass and distribution in 468 men and women aged 18-88 yr. Journal of applied physiology 89: 81-88.
Kabat, et al., (1991). SEQUENCES OF PROTEINS OF IMMUNOLOGICAL INTEREST. Fifth Edition, U.S. Department of Health and Human Services, NIH Publication No. 91 3242. 18 Id.
Khorkova, O. and Wahlestedt, C. (2017). Oligonucleotide therapies for disorders of the nervous system. Nature Biotech 35: 249-263.
Kim, D., et al., (2015). HISAT: a fast spliced aligner with low memory requirements. Nature Methods 12: 357-360.
Koukourikos, K., et al., (2014). Muscle atrophy in intensive care unit patients. Acta informatica medica AIM: journal of the Society for Medical Informatics of Bosnia & Herzegovina: casopis Drustva za medicinsku informatiku BiH 22: 406-410.
Kröger, S., and Schröder, J. E. (2002). Agrin in the developing CNS: new roles for a synapse organizer. News Physiol. Sci. 17(5): 207-212.
Krueger, F. (2015). Trim Galore! [http://www.bioinformatics.babraham.ac.uk/projects/trim_galore/]
Kyba, M. (2016). SKELETAL MUSCLE REGENERATION IN THE MOUSE: METHODS AND PROTOCOLS, Humana Press.
LABORATORY METHODS IN ENZYMOLOGY: DNA, (2013). Jon Lorsch (ed.) Elsevier (ISBN 0124199542).
Lawlor, P. A., et al., (2007). Novel rat Alzheimer's disease models based on AAV-mediated gene transfer to selectively increase hippocampal Aβ levels. Mol. Neurodeg. 2:11.
LEWIN'S GENES XI, (2014). published by Jones & Bartlett Publishers (ISBN-1449659055).
Liu, G., et al., (2005). Adeno-associated virus type 4 (AAV4) targets ependyma and astrocytes in the subventricular zone and RMS. Gene Therapy 12: 1503-1508.
Love, M. I., et al., (2014). Moderated estimation of fold change and dispersion for RNA-seq data with DESeq2. Genome Biology 15: 550.
MacCallum, R. M., et al., (1996). Antibody-antigen interactions: contact analysis and binding site topography. J. Mol. Biol. 262(5): 732-745.
Macintosh, B. R., et al., (2006). Skeletal muscle: form and function. Human Kinetics (Champaign, IL) 2nd ed., 423 p.
Mazhar, S. and Herbst, R. (2012). The formation of complex acetylcholine receptor clusters requires MuSK kinase activity and structural information from the MuSK extracellular domain. Molecular and Cellular Neurosciences 49: 475-486.
McClorey, G. and Banerjee, S., (2018). Cell-Penetrating Peptides to Enhance Delivery of Oligonucleotide-Based Therapeutics. Biomedicines, 6(2): 51.
Mercuri, E. et al., (2018). Nusinersen versus sham control in later-onset spinal muscular atrophy. New England Journal of Medicine, 378: 625-635.
Messeant, J., et al., (2015). MuSK frizzled-like domain is critical for mammalian neuromuscular junction formation and maintenance. J Neuroscience 35: 4926-4941.
Min, H. S., et al., (2020). Systemic Brain Delivery of Antisense Oligonucleotides across the Blood-Brain Barrier with a Glucose-Coated Polymeric Nanocarrier. Angew. Chem. Int. Ed. 59: 8173-8180.
Misener, et al, (1999). Bioinformatics Methods and Protocols (Methods in Molecular Biology, Vol. 132), Humana Press.
Miyagoe-Suzuki, Y., et al., (2017). Skeletal Muscle Cells Generated from Pluripotent Stem Cells. Stem Cells Int. 7824614.
MOLECULAR BIOLOGY AND BIOTECHNOLOGY: A COMPREHENSIVE DESK REFERENCE, (1995). Robert A. Meyers (ed.), published by VCH Publishers, Inc. (ISBN 1-56081-569-8).
Morgan, J. E., et al., (1994). Myogenic cell lines derived from transgenic mice carrying a thermolabile T antigen: a model system for the derivation of tissue-specific and mutation-specific cell lines. Dev Biol. 162: 486-498.
Morris (1996). “Epitope Mapping Protocols”, Methods in Molecular Biology, vol. 66.
Murphy, F. V. and Ramakrishnan, V., (2004). Structure of a purine-purine wobble base pair in the decoding center of the ribosome. Nature Structural and Molecular Biology 11: 1251-1252.
Murphy, M. M., et al., (2011). Satellite cells, connective tissue fibroblasts and their interactions are crucial for muscle regeneration. Development 138: 3625-3637.
Myers, E. W. and Miller, W. (1988). Optimal alignments in linear space. CABIOS 4, 11-17.
Ono, Y., et al., (2011). BMP signaling permits population expansion by preventing premature myogenic differentiation in muscle satellite cells. Cell death and differentiation 18: 222-234.
Otsuka, S., et al, (2011). Delayed intrathecal delivery of RhoA siRNA to the contused spinal cord inhibits allodynia, preserves white matter, and increases serotonergic fiber growth. J Neurotrauma. 28: 1063-1076.
Padlan, E. A., et al., (1995). Identification of specificity-determining residues in antibodies. FASEB J. 9(1): 133-139.
Paul, C. P., et al., (2002). Effective expression of small interfering RNA in human cells. Nature Biotechnol.; 20:505-508
Pawlikowski, B., et al., (2015). Pervasive satellite cell contribution to uninjured adult muscle fibers. Skeletal muscle 5: 42.
Pertea, M., et al., (2016). Transcript-level expression analysis of RNA-seq experiments with HISAT, StringTie and Ballgown. Nature Protocols 11: 1650-1667.
Pimentel, H., et al., (2017). Differential analysis of RNA-seq incorporating quantification uncertainty. Nature Methods 14: 687-690.
Powers, S. K., et al. (2016). Disease-Induced Skeletal Muscle Atrophy and Fatigue. Med. Sci. Sports Exerc. 48(11): 2307-2319.
Prakash, T. P., et al. (2019). Fatty acid conjugation enhances potency of antisense oligonucleotides in muscle. Nucleic Acids Research, 47: 6029-6044.
Randolph, M. E. and Pavlath, G. K. (2015). A muscle stem cell for every muscle: variability of satellite cell biology among different muscle groups. Frontiers in aging neuroscience 7: 190.
Remedio, L., et al., (2016). Diverging roles for Lrp4 and Wnt signaling in neuromuscular synapse development during evolution. Genes & Development 30: 1058-1069.
Remington, (2000). THE SCIENCE AND PRACTICE OF PHARMACY, 20th ed.
Riaz, M., et al., (2015). Differential myofiber-type transduction preference of adeno-associated virus serotypes 6 and 9. Skeletal Muscle 5 (37).
Rigo, F., et al., (2014). Pharmacology of a Central Nervous System Delivered 2′-O-Methoxyethyl-Modified Survival of Motor Neuron Splicing Oligonucleotide in Mice and Nonhuman Primates. J. Pharmacol. Exp. Ther. 350(1): 46-55.
Rodgers, J. T., et al., (2014). mTORC1 controls the adaptive transition of quiescent stem cells from GO to G(Alert). Nature 510: 393-396.
Rupp, F., et al., (1991). Structure and expression of a rat agrin. Neuron. 6 (5): 811-823.
Sacco, A., et al., (2008). Self-renewal and expansion of single transplanted muscle stem cells. Nature 456: 502-506.
Sambasivan, R., et al., (2011). Pax7-expressing satellite cells are indispensable for adult skeletal muscle regeneration. Development 138: 3647-3656.
Sander, J. D. and Joung, J. K. (2014). CRISPR-Cas systems for editing, regulating and targeting genomes. Nature Biotechnology 32: 347-355.
Sartori, R., et al., (2013). BMP signaling controls muscle mass. Nature Genetics 45: 1309-1318.
Schultz, E., et al., (1978). Satellite cells are mitotically quiescent in mature mouse muscle: an EM and radioautographic study. The Journal of Experimental Zoology 206: 451-456.
Seale, P., et al., (2000). Pax7 is required for the specification of myogenic satellite cells. Cell 102: 777-786.
Stantzou, A., et al., (2017). BMP signaling regulates satellite cell-dependent postnatal muscle growth. Development 144: 2737-2747.
Stiegler, A. L., et al., (2006). Crystal structure of the agrin-responsive immunoglobulin-like domains 1 and 2 of the receptor tyrosine kinase MuSK. Journal of molecular biology 364: 424-433.
Stiegler, A. L., et al., (2009). Crystal structure of the frizzled-like cysteine-rich domain of the receptor tyrosine kinase MuSK. J. Mol. Biol. 393: 1-9.
Sui, G., et al., (2002). A DNA vector-based RNAi technology to suppress gene expression in mammalian cells. Proc. Natl. Acad. Sci. USA 99(8): 5515-5520.
THE ENCYCLOPEDIA OF MOLECULAR CELL BIOLOGY AND MOLECULAR MEDICINE, Robert S. Porter et al., (eds.), published by Blackwell Science Ltd., 1999-2012 (ISBN 9783527600908).
THE MERCK MANUAL OF DIAGNOSIS AND THERAPY, (2011). 19th Edition, published by Merck Sharp & Dohme Corp., (ISBN 978-O-911910-19-3).
Till, J. H., et al., (2002). Crystal structure of the MuSK tyrosine kinase: insights into receptor autoregulation. Structure 10: 1187-1196.
Tsen, G., et al., (1995). Agrin is a heparan sulfate proteoglycan. J Biol Chem. 270(7): 3392-3399.
Valenzuela, D. M., et al., (1995). Receptor tyrosine kinase specific for the skeletal muscle lineage: expression in embryonic muscle, at the neuromuscular junction, and after injury. Neuron 15: 573-584.
van Velthoven, C. T. J., et al., (2017). Transcriptional Profiling of Quiescent Muscle Stem Cells In Vivo. Cell reports 21: 1994-2004.
Vaughan, T. J., et al., (1998). Human antibodies by design. Nature Biotechnology, 16: 535-539.
Verma, A. (2018). Recent Advances in Antisense Oligonucleotide Therapy in Genetic Neuromuscular Diseases. Ann. Indian Acad, Neurol. 21(1): 3-8.
Wang, M., et al., (2019). Aminoglycoside Enhances the Delivery of Antisense Morpholino Oligonucleotides In Vitro and in mdx Mice. Mol. Ther. Nucleic Acids, 16: 663-674.
Yilmaz, A., et al., (2016). MuSK is a BMP co-receptor that shapes BMP responses and calcium signaling in muscle cells. Science Signaling 9: ra87.
Yin, H., et al., (2013). Satellite cells and the muscle stem cell niche. Physiological Reviews 93: 23-67.
Yu, J.-Y., et al., (2002). RNA interference by expression of short-interfering RNAs and hairpin RNAs in mammalian cells. Proc. Natl. Acad. Sci. USA, 99: 6047-6052.
Zammit, P. S., et al., (2002). Kinetics of myoblast proliferation show that resident satellite cells are competent to fully regenerate skeletal muscle fibers. Experimental cell research 281: 39-49.
Zammit, P. S., et al., (2004). Muscle satellite cells adopt divergent fates: a mechanism for self-renewal? The Journal of Cell Biology 166: 347-357.
Zhou, X., et al., (2010). Reversal of cancer cachexia and muscle wasting by ActRIIB antagonism leads to prolonged survival. Cell 142: 531-543.

### EQUIVALENTS

Those skilled in the art will recognize or be able to ascertain using no more than routine experimentation, many equivalents to the specific embodiments of the invention described herein. The scope of the present invention is not intended to be limited to the above Description, but rather is as set forth in the following claims:

We claim:

1. A method of treating a subject suffering from one or more features of neuromuscular dysfunction or a muscular dystrophy, the method comprising a step of:
increasing level or activity of a MuSK polypeptide lacking a functional Ig3 domain; and/or
reducing level or activity of a BMP-MuSK polypeptide complex, wherein the MuSK comprises a functional Ig3 domain.

2. A method of increasing muscle regeneration and/or muscle growth, the method comprising a step of:
increasing level or activity of a MuSK polypeptide lacking a functional Ig3 domain; and/or
reducing level or activity of a BMP-MuSK polypeptide complex, wherein the MuSK comprises a functional Ig3 domain.

3. A method of preventing or treating muscle fibrosis, the method comprising a step of:
increasing level or activity of a MuSK polypeptide lacking a functional Ig3 domain; and/or
reducing level or activity of a BMP-MuSK polypeptide complex, wherein the MuSK comprises a functional Ig3 domain.

4. The method of any one of claims 1-3, wherein the method further comprises the step of administering to a subject a pharmaceutical composition that comprises or delivers a MuSK muscle regeneration (MR) agonizing agent.

5. The method of claim 4, wherein the MuSK MR agonizing agent is an agent that downregulates the MuSK Ig3 domain protein expression, the MuSK Ig3 domain gene expression, and/or the MuSK Ig3 activation of BMP signaling, wherein the composition prevents or reduces the accumulation of extracellular matrix within the extracellular space of the muscle.

6. The method of claim 3, wherein the MuSK MR agonizing agent is or comprises a small molecule.

7. The method of claim 4, wherein the MuSK MR agonizing agent is or comprises an antibody agent.

8. The method of claim 4, wherein the MuSK MR agonizing agent is or comprises an oligonucleotide.

9. The method of claim 7, wherein the antibody agent specifically binds to a MuSK polypeptide.

10. The method of claim 9, wherein the antibody agent targets MuSK and specifically binds to the Ig3 domain of a MuSK polypeptide.

11. The method of claim 10, wherein the antibody targeting the Ig3 domain of MuSK protein may bind specifically to the Ig3 domain relative to the Ig1 or Ig2 domains of MuSK.

12. The method of claim 7, wherein the antibody agent is an immunoglobulin molecule comprising four polypeptide chains, e.g., two heavy (H) chains and two light (L) chains.

13. The method of claim 7, wherein the antibody agent is or comprises a monoclonal antibody.

14. The method of claim 7, wherein the antibody agent may be or comprise a polyclonal antibody.

15. The method of claim 8, wherein the MuSK MR agonizing agent is an oligonucleotide.

16. The method of claim 15, wherein the oligonucleotide is a MuSK Ig3-targeted CRISPR/Cas9.

17. The method of claim 15, wherein the oligonucleotide is a MuSK Ig3-targeted siRNA.

18. The method of claim 15, wherein the oligonucleotide is a MuSK Ig3-targeted shRNA.

19. The method of claim 15, wherein the step further comprises increasing the altered splicing of transcripts.

20. The method of claim 19, wherein the altered splicing of transcripts is or comprises altering MuSK splicing.

21. The method of claim 20, wherein the altered MuSK splicing includes production of products having desired and/or improved biological functions, and/or knockdown of undesired product by, modifying splicing products so that undesired biological functions can be suppressed.

22. The method of claim 21, wherein the altered MuSK splicing includes products of transcripts which lack a sequence that encodes MuSK Ig3 domain.

23. The method of claim 22, wherein the splicing product is mRNA.

24. The method of claim 20, wherein the alteration comprises skipping one or more exons.

25. The method of claim 24 wherein the splicing of a transcript is increased in that exon skipping increases levels of mRNA and proteins that have improved beneficial activities compared with absence of exon skipping.

26. The method of claim 24, wherein the splicing of a transcript is increased in that exon skipping lowers levels of mRNA and proteins that have undesired activities compared with absence of exon skipping.

27. The method of claim 26, wherein the splicing of a transcript is increased in that exon skipping lowers levels of mRNA and proteins of MuSK Ig3 domain.

28. The method of claim 24, wherein the skipped one or more exons are in the MuSK Ig3 domain.

29. The method of claim 28, wherein the skipped exon is exon 6 of MuSK Ig3 domain

30. The method of claim 28, wherein the skipped exon is exon 7 of MuSK Ig3 domain

31. The method of claim 28, wherein the skipped exons are exons 6 and 7 of MuSK Ig3 domain.

32. The method of claim 28, wherein the composition comprises oligonucleotide comprising controlled structural elements, e.g., controlled chemical modification, provide unexpected properties.

33. The method of claim 32, wherein the oligonucleotide comprises chemical modifications.

34. The method of claim 33, wherein the chemical modifications comprise one or more types of base modifications, sugar modification, and internucleotidic linkage modifications.

35. The method of claim 34 wherein the chemical modifications comprise sugar modification.

36. The method of claim 15 wherein the sugar modification is 2-MOE modification.

37. A method of inducing MuSK exon skipping by:
contacting a system comprising a population of MuSK primary transcripts with an oligonucleotide that binds to such primary transcripts so that skipping of one or both of exons 6 and 7 is increased.

38. The method of claim 37, wherein the oligonucleotide comprises controlled structural elements, e.g., controlled chemical modification, that provide unexpected properties.

39. The method of claim 38, wherein the oligonucleotide comprises chemical modifications.

40. The method of claim 39, wherein the chemical modifications comprise one or more types of base modifications, sugar modification, and internucleotidic linkage modifications.

41. The method of claim 40, wherein the chemical modifications comprise sugar modification.

42. The method of claim 41, wherein the sugar modification is 2-MOE modification.

43. The method of claim 37, further comprising the step of administering to a subject a pharmaceutically effective amount of a composition that comprises and/or delivers the oligonucleotide to the subject.

44. The method of claim 43, wherein the composition is delivered to the CNS.

45. The method of claim 43, wherein the composition is delivered to the cerebrospinal fluid.

46. The method of claim 43, wherein the compositions is administered to the muscle.

47. The method of claim 43, wherein the composition can be formulated for systemic or localized administration.

48. The method of claim 43, wherein the composition is formulated for delivery by a route selected from intravenous injection, intravenous infusion, intramuscular injection, intrathecal administration, oral administration, buccal administration, inhalation, nasal administration, topical administration, ophthalmic administration or otic administration.

49. The method of claim 48, wherein the composition is formulated for delivery by intramuscular administration.

50. The method of claim 48, wherein the composition is formulated for delivery by intravenous administration.

51. The method of claim 48, wherein the composition is formulated for delivery by oral administration.

52. The method of any one of claims 4-51 wherein the subject is at risk of, or afflicted with, a disease or disorder selected from the group consisting of: neuromuscular dysfunction, neurodegenerative disorder, cardiac disorder, and diseases characterized by muscle wasting.

53. The method of claim 52 wherein the neuromuscular dysfunction is a muscular dystrophy selected from the group consisting of: Becker, Congenital, Distal, Duchenne, Emery-Dreifuss, Facioscapulohumeral, Limb-girdle, Myotonic, and Oculo-pharyngeal muscular dystrophy.

54. The method of claim 52 wherein the cardiac disorder is myocardial infarction or cardiomyopathy.

55. The method of any one of claims 4-51 wherein the subject is in need of enhanced muscle regeneration and/or muscle growth following a condition selected from the group consisting of: surgery, trauma and prolonged immobilization.

56. The method of claim 55, wherein the prolonged immobilization results from bed-rest or casting.

57. The method of any one of claims 4-51, wherein the subject is at risk of, or afflicted with, sarcopenia.

58. The method of any one of claims 4-51, wherein the subject is at risk of, or afflicted with, muscle fibrosis resulting from a disease or condition selected from the group consisting of: trauma, heritable disease, muscle disorder, and aging.

59. The method of claim 58 wherein the trauma is the result of a condition selected from the group consisting of: radiation treatment, crush injury, laceration, and amputation.

60. The method of claim 58, wherein the heritable disease or muscle disorder selected from the group consisting of: Congenital Muscular Dystrophy, Duchenne Muscular Dystrophy, Becker's Muscular Dystrophy; Amyotrophic Lateral Sclerosis (ALS), and age-associate sarcopenia.

61. A population of cells that has been exposed to a MuSK muscle regeneration agonizing agent, such that, level or percentage of cells characterized by myogenic marker(s) has been increased within the population relative to that observed absent the exposure.

62. The population of claim 61, wherein the myogenic marker(s) is or are selected from the group consisting of Pax7, MyoD, myogenin, and MERGE, and combinations thereof.

63. The population of claim 61, wherein the increase in level or percentage is an increase of at least 10%, 20%, 30%, 40%, 50%, 60%, 70%, 80%, 90%, 95%, or 95% greater relative to that observed absent the exposure.

64. The population of claim 61, wherein the muscular marker(s) are indicative of an activated muscle (e.g., satellite) cells.

65. A method comprising a step of:
contacting an original population of cells that is or comprises muscular progenitor cells with a MuSK muscle regeneration agonizing agent to generate a resulting population, the contacting being performed under conditions and for a time sufficient that level or percentage of cells characterized by muscular marker(s) is significantly higher in the resulting population than in the original population.

66. The method of claim 65, wherein the step of contacting occurs in vivo.

67. The method of claim 66, wherein the step of contacting occurs in an adult human.

68. The method of claim 66 or 67, wherein the step of contacting occurs at a site within muscle tissue.

69. The method of claim 66 or 67, wherein the step of contacting occurs ex vivo.

70. The method of claim 69, wherein the population of cells was obtained from a subject suffering from a neuromuscular dysfunction, neurodegenerative disorder, cardiac disorder, or a disease characterized by muscle wasting.

71. The method of claim 69, further comprising administering the resulting population to the subject.

72. The method of claim 65, wherein the neural marker(s) is or are selected from the group consisting of Pax7, MyoD, myogenin, and MERGE, and combinations thereof.

73. The method of claim 65, wherein the muscular marker(s) are indicative of an activated muscle (e.g., satellite) cell.

74. A method of characterizing a MuSK muscle regeneration agonizing agent, the method comprising one or more of:
assessing ability to reduce MuSK-Ig3-BMP complex formation (dependencies re prevent formation, disrupt formed, assess direct binding to Ig3 and/or BMP, concentration dependence etc.);
assessing ability to alter splicing pattern of primary MuSK transcripts;
assessing ability to inhibit expression (dependent includes induce degradation, inhibit translation, etc.) of transcript (e.g., including Ig3);
assessing the ability to increase expression of MuSK transcripts lacking a sequence encoding the Ig3 domain;
assessing the ability to increase level of MuSK polypeptide lacking functional Ig3; and
assessing the ability to impact characteristics of cells in a population.

75. The method of claim 74, wherein the MuSK MR agonizing agent is an oligonucleotide.

76. The method of claim 75, wherein the oligonucleotide comprising at least one modification.

77. The method of claim 75 or claim 76, wherein the oligonucleotide, when administered to a subject, alters splicing activity of primary MuSK transcripts so that skipping of one or both of exons 6 and 7 is increased.

78. A genetically modified mouse that comprises in its genome:
a sequence encoding the MuSK, wherein the sequence encoding MuSK does not include the span of nucleotides from (in 5′ to 3′ order) exon 6 to exon 7;
wherein the genetically modified mouse is not capable of expressing the full length MuSK transcript or producing full length MuSK protein.

79. The genetically modified mouse of claim 78, wherein the mouse is not capable of expressing MuSK protein that includes the amino acid sequence in SEQ ID NO: 2.

80. The genetically modified mouse of claim 78, wherein the mouse is capable of expressing MuSK transcript encoding a MuSK protein lacking an Ig3 domain.

81. The genetically modified mouse of claim 78, wherein the mouse exhibits increased muscle regeneration, compared to a mouse that is able to express the full length MuSK transcript or produce full length MuSK protein.

82. The genetically modified mouse of claim 81, wherein the increased muscle regeneration comprises an increase is motor function.

83. The genetically modified mouse of claim 78, wherein the mouse is genetically modified by removing a span of nucleotides from (in 5′ to 3′ order) exon 6 to exon 7 in the sequence encoding MuSK using a CRISPR/Cas9 system.

84. The genetically modified mouse of claim 83, wherein the CRISPR/Cas9 system includes gDNA that targets regions within exon6 and/or exon7 of the MuSK gene sequence.

85. The genetically modified mouse of claim 84, wherein the gDNA targeting sequences comprise SEQ ID NOs: 3-6.
